(19) United States
(12) Patent Application Publication
(10) Pub. No.: US 2020/0340060 A1
(43) Pub. Date: Oct. 29, 2020

(54) COMPOSITIONS AND METHODS FOR IDENTIFYING AND TREATING LIVER DISEASES AND MONITORING TREATMENT OUTCOMES

(71) Applicant: Gilead Sciences, Inc., Foster City, CA (US)

(72) Inventors: Andrew Nicholas Billin, San Mateo, CA (US); Jen-Chieh Chuang, Foster City, CA (US); Ren Y. Xu, San Mateo, CA (US)

(21) Appl. No.: 16/763,495

(22) PCT Filed: Nov. 9, 2018
(86) PCT No.: PCT/US18/60106

§ 371 Date: May 12, 2020

Related U.S. Application Data
(60) Provisional application No. 62/585,421, filed on Nov. 13, 2017.
(60) Provisional application No. 62/739,778, filed on Oct. 1, 2018.

(51) Int. Cl. C12Q 1/6883; G01N 33/68

(52) U.S. Cl. CPC C12Q 1/6883; G01N 33/6893; G01N 2800/56; A61K 45/06; G01N 2800/52; C12Q 2600/112; C12Q 2600/158; G01N 2800/085

(57) ABSTRACT

The present disclosure relates generally to methods of treating human patients suffering from a liver disease or condition. The disclosure also provides diagnostic methods for determining the stage or status of the liver disease or condition and monitoring methods for assessing the effectiveness of a treatment, using serum protein, metabolites or bile acid markers.

### CROSS REFERENCE TO RELATED APPLICATIONS

This application is a U.S. National Stage filing of PCT Application US2018/060106 filed on Nov. 9, 2018 which claims the benefit under 35 U.S.C. § 119(e) of United States Provisional Application Nos. 62/585,421, filed Nov. 13, 2017, and 62/739,778, filed Oct. 1, 2018, the content of each is incorporated by reference in their entirety into the present disclosure.

### BACKGROUND

NASH represents a significant and growing unmet medical need with no currently approved therapies. An estimated 16 million adults in the United States have NASH. Approximately 25% of patients diagnosed with NASH have advanced liver fibrosis, which is associated with increased morbidity and mortality.

Biopsy is considered a standard for assessments of liver health including NASH. However, biopsy is an invasive technique that is not ideal for many patients. Non-invasive methods may provide advantages for diagnosing, monitoring, or predicting NASH. Described herein are non-invasive markers associated with NASH or a related presentation.

### SUMMARY

The present disclosure is based on the evaluation of protein and bile acid markers whose levels correlate with the stage, status or treatment outcome of liver diseases or conditions. In one embodiment, provided is a method for determining the stage of liver fibrosis in a human subject in need thereof, comprising measuring the expression levels of one or more proteins, selected from Tables 1A-1F and 11A-11D, in a biological sample isolated from the human subject; and determining the stage of liver fibrosis in the human subject based on the expression levels.

Another embodiment provides a method for providing biological information for diagnosing liver fibrosis in a human subject, comprising measuring the expression levels of two or more proteins, selected from Tables 1A-1F and 11A-11D, in a biological sample isolated from the human subject.

Another embodiment provides a method for assessing the effect of a treatment in a patient suffering from liver fibrosis and having received the treatment, comprising measuring the expression levels of one or more proteins, selected from Tables 3A-3D and 12, in a biological sample isolated from the patient; and assessing the effect of the treatment by comparing the expression levels to baseline expression levels obtained from the patients prior to the treatment.

Another embodiment provides a method for providing biological information for assessing the effect of a treatment in a patient suffering from liver fibrosis and having received the treatment, comprising measuring the expression levels of two or more proteins, selected from Tables 3A-3D and 12, in a biological sample isolated from the patient.

Various methods are also provided for treating patients having a liver disease or condition after suitable analysis, as disclosed here, has been performed.

### BRIEF DESCRIPTION OF THE DRAWINGS

FIG. 1 shows proteins whose expression levels in the serum significantly correlated with NASH disease severity.

FIG. 2 is a Venn diagram showing the overlaps between fibrosis stage and NAS components for the protein markers.

FIG. 3 shows that the diagnostic ability of fibrosis stage at baseline using combined biomarkers is increased compared to the individual best markers.

FIG. 4-7 show the performance of multivariate protein markers for monitoring improvement of clinical parameters, CRN fibrosis stage (FIG. 4), steatosis (FIG. 5), lobular inflammation (FIG. 6), and hepatic ballooning (FIG. 7).

FIG. 8 presents a chart showing that serum bile acid profiles are significantly altered in NASH subjects with different fibrosis stage.

FIG. 9 shows that Serum C4 (7α-hydroxy-4-cholesten-3-one) levels are increased in F2/F3 NASH subjects but are decreased in F4 and decompensated subjects. **p<0.01, ***p<0.001, ****p<0.0001.

FIG. 10 shows unique and overlapping potential secreted/leaked from multiple databases.

FIG. 11. NASH secretome workflow.

FIG. 12. Diagnosis of severe fibrosis or cirrhosis using hepatic RNA levels of top combined and individual secretome candidates.

FIG. 13. Secretome candidates demonstrated significant association between circulating protein levels and liver fibrosis in 1497 and sim study samples (F2-4).

FIG. 14-15 show the significant correlation between circulating proteins GDF-15 and certain NASH stages and characteristics.

FIG. 16-18 show the significant correlation between circulating proteins CD163 and certain NASH stages and characteristics.

FIG. 19 is a Venn diagram showing overlapped metabolite markers for different NASH phenotypes.

It will be recognized that some or all of the figures are schematic representations for purpose of illustration.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. The headings provided herein are for convenience only and not as limitation in any way.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “some embodiments,” “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “sample” refers generally to a fluid from a human. Non-limiting examples of a sample include: bile, blood, blood plasma, serum, breast milk, feces, pus, saliva, sebum, semen, sweat, tears, urine, and vomit. In some embodiments, the sample is serum.

As used herein, the term “subject” refers to a mammalian subject. Exemplary subjects include, but are not limited to humans, monkeys, dogs, cats, mice, rats, cows, horses, goats and sheep. In some embodiments, the subject has a liver disease or condition and can be treated as described herein.

As used herein, the term “suspected” when referencing a patient refers to the potential for a patient to have a certain liver disease or condition based on a correlate.

As used herein, the term “treatment,” “treating,” or similar language refers to a process to (1) delay onset of a disease that is causing clinical symptoms; (2) inhibiting a disease, that is, arresting the development of clinical symptoms; and/or (3) relieving the disease, that is, causing the regression of clinical symptoms or the severity thereof.

As used herein, the term “liver disease or condition” refers any one or more of the following: liver fibrosis, alcoholic hepatitis, nonalcoholic steatohepatitis (NASH), non-alcoholic fatty liver disease (NAFLD), or liver inflammation.

As used herein, the term “NAS” refers to the NAFLD Activity Score, which is a scoring system for NAFLD.

### Identification and Treatment of Liver Diseases or Conditions

The experimental examples of the present disclosure identified non-invasive markers from serum samples that can be used to diagnose liver diseases or conditions, such as liver fibrosis, nonalcoholic steatohepatitis (NASH), non-alcoholic fatty liver disease (NAFLD), and liver inflammation. More specifically, the instant inventors demonstrated that the expression levels of certain protein markers, individually or in combination, significantly correlated with the stage or status of the liver disease or condition. In addition, the serum C4 (7α-hydroxy-4-cholesten-3-one) levels, which were measured to reflect hepatic bile acid biosynthesis, correlated with fibrosis stages and development of cirrhosis.

It is to be understood that information obtained using the diagnostic assays described herein may be used alone or in combination with other information, such as, but not limited to, genotypes or expression levels of other proteins, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more proteins as disclosed herein are used in a panel of proteins, each of which contributes to the final diagnosis, prognosis or treatment.

Protein markers have also been identified that correlate with clinical improvements following a treatment. These markers, therefore, can be used to monitor the treatment of patients. For example, when the markers show that a treatment has been effective in a patient, the patient may be instructed to continue the treatment. By contrast, if the markers show that no desired improvements have been achieved with the treatment, then a new treatment (e.g., a new medicine or a higher dose) may be used.

In accordance with one embodiment of the present disclosure, therefore, provided is a method of determining the stage or status of liver disease or condition in a human subject, e.g., one that is suspected to have a liver disease or condition. The method, in some embodiments, entails measuring the expression levels of one or more proteins/genes, selected from Tables 1A-1F, in a biological sample isolated from the human subject and determining the stage of liver fibrosis in the human subject based on the expression levels. In some embodiments, the determination comprises comparing the expression levels to reference levels. In some embodiments, the reference levels are obtained from a human subject not suffering from liver fibrosis.

For instance, Table 1A shows that the protein marker “Collectin Kidney 1” has expression levels 5987.2, 4903.3, 8174.95, 11267.55, and 15604.5 (unit: relative fluorescent unit (RFU)), at CRN fibrosis stages 0-4, respectively in the test sample. When a new subject is tested and the expression level of Collectin Kidney 1 is 11000 (RFU, normalized), a determination can be made that this new subject likely has a liver fibrosis at CRN fibrosis stage 3.

In some embodiments, the proteins are selected from Table 1A or Table 2A. The diagnostic variable, in this aspect, is a CRN fibrosis stage. In some embodiments, the proteins are selected from Table 1B or 2B, and the diagnostic variable is an Ishak Fibrosis stage. In some embodiments, the proteins are selected from Table 1C or 2C, and the diagnostic variable is a NAS score. In some embodiments, the proteins are selected from Table 1D or 2D, and the diagnostic variable is a steatosis. In some embodiments, the proteins are selected from Table 1E or 2E, and the diagnostic variable is lobular inflammation (LI). In some embodiments, the proteins are selected from Table 1F or 2F, and the diagnostic variable is hepatic ballooning (HB).

In some embodiments, the expression levels of at least two, or three, four, five, six, seven, eight, nine or ten proteins are measured.

It can be seen from the tables that some protein markers are associated with multiple diagnostic variables while some are unique to a particular variable (see summary in Table A). For instance, 6Ckine, BSSP4, IL-8, LIF sR, LTBP4, MIC-1, SAP, SEM6B, SLAF7, and Spondin-1 are unique to CRN fibrosis and HSP 70 is common to all five parameters in Table 5. Accordingly, a suitable expression level of 6Ckine may indicate a particular CRN fibrosis stage, while a suitable expression level of HSP 70 implicates a particular stage or status for all five variables.

As shown in the multivariant analysis of Example 1, a group of seven protein markers, when used in combination, had even greater diagnostic capability. These seven protein markers include C7 (Complement component 7), CL-K1 (Collectin Kidney 1), IGFBP7 (Insulin-like growth factor binding protein 7), Spondin 1 (RSPO1), IL-5Ra (UniProt: Q01344; Interleukin 5 receptor subunit alpha), MMP-7 (Matrix metallopeptidase 7) and TSP2 (Thrombospondin-2). In some embodiments, at least two of the seven markers are used. In some embodiments, at least three, four, five, or six, or all seven of the seven markers are used. In some embodiments, the selected markers include at least Spondin 1. In some embodiments, the selected markers include at least IL-5Ra. In some embodiments, the selected markers include at least MMP 7.

In some embodiments, as Example 2 has demonstrated, the serum levels of C4 (7α-hydroxy-4-cholesten-3-one) correlate with the stage and status of liver diseases as well, which can individually, or in combination with other markers such as those disclosed herein, for the purpose of making diagnosis for liver diseases or conditions.

In addition or independently, the disease assessment can be made with information obtained through conventional or non-conventional methods. In one embodiment, a method is provided for identifying a non-alcoholic steatohepatitis (NASH) patient as likely suffering advanced fibrosis. The method entails, in one embodiment, measuring the AST level, ALT level and platelet count for the NASH patient and calculating a Fibrosis-4 (FIB-4) index, measuring the serum concentrations of tissue inhibitor of metalloproteinases 1 (TIMP-1), amino-terminal propeptide of type III procollagen (PIIINP) and hyaluronic acid (HA) for the NASH patient and calculating an enhanced liver fibrosis (ELF) score, or conducting a transient elastographic (Fibroscan) of the NASH patient to determine a Fibroscan score, and comparing the FIB-4 index and either or both of the ELF score and the Fibroscan score to reference values, and identifying the NASH patient as likely suffering from advanced fibrosis based on the comparison.

In some embodiments, the FIB-4 index and the ELF score are determined. In some embodiments, the FIB-4 index, the ELF score and the Fibroscan score are determined. In some embodiments, no invasive measurements are made to the NASH patient. In one embodiment, an assessment of the FIB-4 or ELF score is made and a patient is treated with one or more therapeutic agents selected from an ASK1 inhibitor, an ACC inhibitor, or an FXR agonist. In one embodiment, an assessment of the FIB-4 or ELF score is made and a patient is treated with selonsertib.

As demonstrated in Example 6, metabolites measured in serum samples of an individual can also be used as biomarkers for assessing disease stages. In accordance with one embodiment of the present disclosure, therefore, provided is a method of determining the stage or status of liver disease or condition in a human subject, e.g., one that is suspected to have a liver disease or condition. The method, in some embodiments, entails measuring the levels of one or more metabolites, selected from Tables 15-27, in a biological sample (e.g., serum) obtained from the human subject and determining the stage of liver fibrosis in the human subject based on the levels. In some embodiments, the determination comprises comparing the levels to reference levels. In some embodiments, the reference levels are obtained from a human subject not suffering from liver fibrosis.

In some embodiments, the metabolites are selected from Table 15. The diagnostic variable, in this aspect, is a CRN fibrosis stage. In some embodiments, the metabolites are selected from Table 16, and the diagnostic variable is a NAS score. In some embodiments, the metabolites are selected from Table 17, and the diagnostic variable is a steatosis. In some embodiments, the metabolites are selected from 19, and the diagnostic variable is lobular inflammation (LI). In some embodiments, the metabolites are selected from Table 18, and the diagnostic variable is hepatic ballooning (HB).

In some embodiments, the expression levels of at least two, or three, four, five, six, seven, eight, nine or ten metabolites are measured.

In some embodiments, determination of a liver disease or condition in a patient is followed by treatment as described herein. In other embodiments, determination of a liver disease or condition is conducted during the course of treatment.

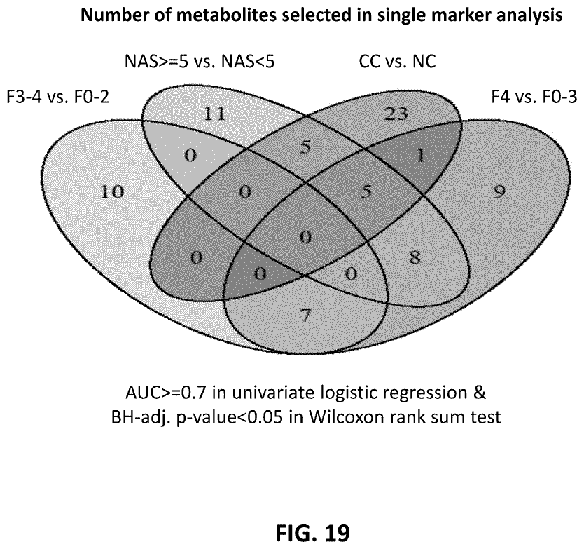

As demonstrated in Example 6 and FIG. 19, different panels of biomarkers can differentiate patients with different presentation of disease. In one embodiment, such a biomarker panel as described in Example 6 may be used to distinguish patients with the following presentations: F0-2 versus F3-4, NAS>=5 versus NAS<5, cryptogenic cirrhotics versus non-cryptogenic cirrhotics, and F4 versus F0-3 patients. Once the presentation of a patient is determined from obtaining a biological sample including the panel described in Example 6, the patient may be treated by one or more therapeutic agents as described herein.

### Monitoring of Clinical Improvements

As demonstrated in Example 1, protein markers have been identified that correlate well with the improvement of certain clinical endpoints. These protein markers, therefore, can be used to monitor the effectiveness of a treatment in a patient.

In one embodiment, therefore, the present disclosure provides a method for assessing the effect of a treatment in a patient suffering from a liver disease or condition and having received the treatment. In some embodiments, the method entails measuring the expression levels of one or more proteins, selected from Tables 3A-D and 12, in a biological sample isolated from the patient; and assessing the effect of the treatment by comparing the expression levels to baseline expression levels obtained from the patients prior to the treatment.

For instance, for a patient having liver fibrosis, if the KYNU levels goes down 30% after the treatment, the patient is likely responsive to the treatment as this change suggests improvement of steatosis. By contrast, if the decrease of KYNU level is only about 5%, this patient is then likely not responding to the treatment, and a new treatment option (e.g., different drug, longer treatment or higher dose) is warranted.

Each of the clinical endpoints/variables (steatosis, lobular inflammation, hepatic ballooning, and CRN fibrosis stage) has a corresponding list of protein markers for monitoring its improvement. In one embodiment, the clinical endpoint is improvement of steatosis, and the protein marker is selected from Table 3A, Table 4A or Table 12. In one embodiment, the clinical endpoint is improvement of lobular inflammation, and the protein marker is selected from Table 3B, Table 4B or Table 12. In one embodiment, the clinical endpoint is improvement of hepatic ballooning, and the protein marker is selected from Table 3C, Table 4C or Table 12. In one embodiment, the clinical endpoint is improvement of CRN fibrosis stage, and the protein marker is selected from Table 3D or Table 4D.

Multivariate marker groups are also identified for each clinical endpoints, which are summarized in Table B. In one embodiment, the clinical endpoint is improvement of CRN fibrosis stage and the protein markers are two, three, four, five, six or more, or all seven selected from pTEN (P60484), CD70 (P32970), Caspase-2 (P42575), Cathepsin H (P09668), LAG-1 (Q8NHW4), PDXK (O00764), and GITR (Q9Y5U5).

In one embodiment, the clinical endpoint is improvement of steatosis and the protein markers are two, three, four, five, six, seven, eight, nine, ten, eleven or more, or all twelve selected from Integrin a1b1 (P56199, P05556), Nectin-like protein 2 (Q9BY67), PDGF Rb (P09619), LRP8 (Q14114), CD30 Ligand (P32971), Lumican (P51884), SAP (P02743), YKL-40 (P36222), sTie-2 (Q02763), HSP 90a/b (P07900 P08238), TSP2 (P35442), and YES (P07947).

In one embodiment, the clinical endpoint is improvement of lobular inflammation and the protein markers are two, three, four, five or more, or all five selected from HSP 90a/b (P07900 P08238), Aminoacylase-1 (Q03154), FCG3B (O75015), M-CSF R (P07333), and Keratin 18 (P05783).

In one embodiment, the clinical endpoint is improvement of hepatic ballooning and the protein markers are two, three, four, five, six or more, or all seven selected from Fibronectin (P02751), Thyroxine-Binding Globulin (P05543), FGF23 (Q9GZV9), LG3BP (Q08380), Heparin cofactor II (P05546), Protein C (P04070) and STAT3 (P40763).

### Kits and Panels

The present disclosure also provides kits, packages and diagnostic panels for use in methods of various embodiments. For instance, the kits may include antibodies, nucleotide probes or primers and other reagents for measuring the protein or mRNA expression of various lists of proteins (e.g., Tables 1A-1F, 2A-2F, 3A-3D, 4A-4D, 11A-11D, 12, A, and B) as disclosure herein. In another embodiment, the kit or package further includes a suitable therapy.

### Panel Testing

Various embodiments disclosure above include multiple protein markers, the measurement of which can be conducted together (simultaneously or sequentially). Such testing will provide information for suitable diagnosis, prognosis, and clinical monitoring, without limitation.

One embodiment provides a method for providing biological information for diagnosing a liver disease or condition in a human subject, comprising measuring the expression levels of two, three, four, five, six, seven, eight, nine, ten, fifty, twenty or more proteins, selected from Tables 1A-1F, Tables 2A-2F or Table 11A-11D, in a biological sample isolated from the human subject. In some embodiments, the proteins are selected from Complement component 7 (C7), Collectin Kidney 1 (CL-K1), Insulin-like growth factor binding protein 7 (IGFBP7), Spondin-1(RSPO1), Interleukin 5 receptor subunit alpha (IL5-Ra), Matrix metallopeptidase (MMP-7), and Thrombospondin-2 (TSP2). In some embodiments, the measurement is carried out for no more than 20, 25, 30, 35, 40, or 50 proteins.

Another embodiment provides a method for providing biological information for determining the CRN (Nonalcoholic Steatohepatitis Clinical Research Network) fibrosis stage in a human subject, comprising measuring the expression levels of two, three, four, five, six, seven, eight, nine, ten, fifty, twenty or more proteins, selected from Tables 1A, 2A or 11A, in a biological sample isolated from the human subject. In some embodiments, the measurement is carried out for no more than 20, 25, 30, 35, 40, or 50 proteins.

Another embodiment provides a method for providing biological information for determining the Ishak fibrosis stage in a human subject, comprising measuring the expression levels of two, three, four, five, six, seven, eight, nine, ten, fifty, twenty or more proteins, selected from Tables 1B or 2B, in a biological sample isolated from the human subject. In some embodiments, the measurement is carried out for no more than 20, 25, 30, 35, 40, or 50 proteins.

Another embodiment provides a method for providing biological information for determining the NAS (nonalcoholic fatty liver disease (NAFLD) activity score) in a human subject, comprising measuring the expression levels of two, three, four, five, six, seven, eight, nine, ten, fifty, twenty or more proteins, selected from Tables 1C, 2C or 11B, in a biological sample isolated from the human subject. In some embodiments, the measurement is carried out for no more than 20, 25, 30, 35, 40, or 50 proteins.

Another embodiment provides a method for providing biological information for characterizing steatosis in a human subject, comprising measuring the expression levels of two, three, four, five, six, seven, eight, nine, ten, fifty, twenty or more proteins, selected from Tables 1D or 2D, in a biological sample isolated from the human subject. In some embodiments, the measurement is carried out for no more than 20, 25, 30, 35, 40, or 50 proteins.

Another embodiment provides a method for providing biological information for characterizing lobular inflammation in a human subject, comprising measuring the expression levels of two, three, four, five, six, seven, eight, nine, ten, fifty, twenty or more proteins, selected from Tables 1E, 2E or 11C, in a biological sample isolated from the human subject. In some embodiments, the measurement is carried out for no more than 20, 25, 30, 35, 40, or 50 proteins.

Another embodiment provides a method for providing biological information for characterizing hepatic ballooning in a human subject, comprising measuring the expression levels of two, three, four, five, six, seven, eight, nine, ten, fifty, twenty or more proteins, selected from Tables 1F, 2F or 11D, in a biological sample isolated from the human subject. In some embodiments, the measurement is carried out for no more than 20, 25, 30, 35, 40, or 50 proteins.

In some embodiments, the method further comprises making a diagnosis based on the biological information. In some embodiments, the method further comprises prescribing or administering to the human subject a therapy according to the diagnosis.

One embodiment provides a method for providing biological information for assessing the effect of a treatment in a patient suffering from liver disease or condition and having received the treatment, comprising measuring the expression levels of two, three, four, five, six, seven, eight, nine, ten, fifty, twenty or more proteins, selected from Tables 3A-3D and 12, in a biological sample isolated from the patient. In some embodiments, the proteins are selected from Phosphatase and tensin homolog (PTEN), CD70, Caspase 2, Cathepsin H (CTSH), Sphingosine N-acyltransferase (LAG-1), Pyridoxal kinase (PDXK), and Glucocorticoid-induced TNFR-related protein (GITR). In some embodiments, the measurement is carried out for no more than 20, 25, 30, 35, 40, or 50 proteins.

Another embodiment provides a method for providing biological information for assessing whether a liver disease or condition patient exhibits improvement on steatosis following a treatment, comprising measuring the expression levels of two, three, four, five, six, seven, eight, nine, ten, fifty, twenty or more proteins, selected from Tables 3A, 4A or 12, in a biological sample isolated from the human subject. In some embodiments, the proteins are selected from Integrin a1b1 (P56199, P05556), Nectin-like protein 2 (Q9BY67), PDGF Rb (P09619), LRP8 (Q14114), CD30 Ligand (P32971), Lumican (P51884), SAP (P02743), YKL-40 (P36222), sTie-2 (Q02763), HSP 90a/b (P07900 P08238), TSP2 (P35442), and YES (P07947). In some embodiments, the measurement is carried out for no more than 20, 25, 30, 35, 40, or 50 proteins.

Another embodiment provides a method for providing biological information for assessing whether a liver disease or condition patient exhibits improvement on lobular inflammation following a treatment, comprising measuring the expression levels of two, three, four, five, six, seven, eight, nine, ten, fifty, twenty or more proteins, selected from Tables 3B, 4B or 12, in a biological sample isolated from the human subject. In some embodiments, the proteins are selected from HSP 90a/b (P07900 P08238), Aminoacylase-1 (Q03154), FCG3B (O75015), M-CSF R (P07333), and Keratin 18 (P05783). In some embodiments, the measurement is carried out for no more than 20, 25, 30, 35, 40, or 50 proteins.

Another embodiment provides a method for providing biological information for assessing whether a liver disease or condition patient exhibits improvement on hepatic ballooning following a treatment, comprising measuring the expression levels of two, three, four, five, six, seven, eight, nine, ten, fifty, twenty or more proteins, selected from Tables 3C, 4C or 12, in a biological sample isolated from the human subject. In some embodiments, the proteins are selected from Fibronectin (P02751), Thyroxine-Binding Globulin (P05543), FGF23 (Q9GZV9), LG3BP (Q08380), Heparin cofactor II (P05546), Protein C (P04070) and STAT3 (P40763). In some embodiments, the measurement is carried out for no more than 20, 25, 30, 35, 40, or 50 proteins.

Another embodiment provides a method for providing biological information for assessing whether a liver disease or condition patient exhibits improvement on CRN fibrosis stage following a treatment, comprising measuring the expression levels of two, three, four, five, six, seven, eight, nine, ten, fifty, twenty or more proteins, selected from Tables 3D or 4D, in a biological sample isolated from the human subject. In some embodiments, the proteins are selected from pTEN (P60484), CD70 (P32970), Caspase-2 (P42575), Cathepsin H (P09668), LAG-1 (Q8NHW4), PDXK (O00764), and GITR (Q9Y5U5). In some embodiments, the measurement is carried out for no more than 20, 25, 30, 35, 40, or 50 proteins.

In some embodiments, the method further comprises making a treatment assessment based on the biological information. In some embodiments, the method further comprises prescribing or administering to the human subject a therapy according to the diagnosis.

### Treatment Methods and Uses

Upon obtaining information relating to the diagnosis of a liver disease or condition, or confirmation of effectiveness of a treatment, the present disclosure further provides suitable treatment methods or uses to the patient. In some embodiments, the patient has been analyzed accordingly any embodiment of the present disclosure, with one or more protein markers, optionally with other markers or clinical tests. In some embodiments, the treatment uses one or more of the following therapeutic agents.

### Therapeutic Agents

In some embodiments, one or more therapeutic agents include, and are not limited to, a compound disclosed herein is administered in combination with one or more additional therapeutic agents to treat or prevent a disease or condition disclosed herein. In some embodiments, the one or more additional therapeutic agents are a(n) ACE inhibitor, Acetyl CoA carboxylase inhibitor, Adenosine A3 receptor agonist, Adiponectin receptor agonist, AKT protein kinase inhibitor, AMP-activated protein kinases (AMPK), Amylin receptor agonist, Angiotensin II AT-1 receptor antagonist, Autotaxin inhibitors, Bioactive lipid, Calcitonin agonist, Caspase inhibitor, Caspase-3 stimulator, Cathepsin inhibitor, Caveolin 1 inhibitor, CCR2 chemokine antagonist, CCR3 chemokine antagonist, CCR5 chemokine antagonist, Chloride channel stimulator, CNR1 inhibitor, Cyclin D1 inhibitor, Cytochrome P450 7A1 inhibitor, DGAT1/2 inhibitor, Dipeptidyl peptidase IV inhibitor, Endosialin modulator, Eotaxin ligand inhibitor, Extracellular matrix protein modulator, Farnesoid X receptor agonist, Fatty acid synthase inhibitors, FGF1 receptor agonist, Fibroblast growth factor (FGF-15, FGF-19, FGF-21) ligands, Galectin-3 inhibitor, Glucagon receptor agonist, Glucagon-like peptide 1 agonist, G-protein coupled bile acid receptor 1 agonist, Hedgehog (Hh) modulator, Hepatitis C virus NS3 protease inhibitor, Hepatocyte nuclear factor 4 alpha modulator (HNF4A), Hepatocyte growth factor modulator, HMG CoA reductase inhibitor, IL-10 agonist, IL-17 antagonist, Ileal sodium bile acid cotransporter inhibitor, Insulin sensitizer, integrin modulator, intereukin-1 receptor-associated kinase 4 (IRAK4) inhibitor, Jak2 tyrosine kinase inhibitor, Klotho beta stimulator, ketohexokinase inhibitors such as PF-06835919, 5-Lipoxygenase inhibitor, Lipoprotein lipase inhibitor, Liver X receptor, LPL gene stimulator, Lysophosphatidate-1 receptor antagonist, Lysyl oxidase homolog 2 inhibitor, Matrix metalloproteinases (MMPs) inhibitor, MEKK-5 protein kinase inhibitor, Membrane copper amine oxidase (VAP-1) inhibitor, Methionine aminopeptidase-2 inhibitor, Methyl CpG binding protein 2 modulator, MicroRNA-21(miR-21) inhibitor, Mitochondrial uncoupler such as nitazoxanide, Myelin basic protein stimulator, NACHT LRR PYD domain protein 3 (NLRP3) inhibitor, NAD-dependent deacetylase sirtuin stimulator, NADPH oxidase inhibitor (NOX), Nicotinic acid receptor 1 agonist, P2Y13 purinoceptor stimulator, PDE 3 inhibitor, PDE 4 inhibitor, PDE 5 inhibitor, PDGF receptor beta modulator, Phospholipase C inhibitor, PPAR alpha agonist, PPAR delta agonist, PPAR gamma agonist, PPAR gamma modulator, Protease-activated receptor-2 antagonist, Protein kinase modulator, Rho associated protein kinase inhibitor, Sodium glucose transporter-2 inhibitor, SREBP transcription factor inhibitor, STAT-1 inhibitor, Stearoyl CoA desaturase-1 inhibitor, Suppressor of cytokine signalling-1 stimulator, Suppressor of cytokine signalling-3 stimulator, Transforming growth factor β (TGF-β), Transforming growth factor β activated Kinase 1 (TAK1), Thyroid hormone receptor beta agonist, TLR-4 antagonist, Transglutaminase inhibitor, Tyrosine kinase receptor modulator, GPCR modulator, nuclear hormone receptor modulator, WNT modulators, or YAP/TAZ modulator.

Non-limiting examples of the one or more additional therapeutic agents include:

ACE inhibitors, such as enalapril;

Acetyl CoA carboxylase (ACC) inhibitors, such as DRM-01, gemcabene, PF-05175157, and QLT-091382;

Adenosine receptor agonists, such as CF-102, CF-101, CF-502, and CGS21680;

Adiponectin receptor agonists, such as ADP-355;

Amylin/calcitonin receptor agonists, such as KBP-042;

AMP activated protein kinase stimulators, such as 0-304;

Angiotensin II AT-1 receptor antagonists, such as irbesartan;

Autotaxin inhibitors, such as PAT-505, PAT-048, GLPG-1690, X-165, PF-8380, and AM-063;

Bioactive lipids, such as DS-102;

Cannabinoid receptor type 1 (CNR1) inhibitors, such as namacizumab and GWP-42004;

Caspase inhibitors, such as emricasan;

Pan cathepsin B inhibitors, such as VBY-376;

Pan cathepsin inhibitors, such as VBY-825;

CCR2/CCR5 chemokine antagonists, such as cenicriviroc;

CCR2 chemokine antagonists, such as propagermanium;

CCR3 chemokine antagonists, such as bertilimumab;

Chloride channel stimulators, such as cobiprostone;

Diglyceride acyltransferase 2 (DGAT2) inhibitors, such as IONIS-DGAT2Rx;

Dipeptidyl peptidase IV inhibitors, such as linagliptin;

Eotaxin ligand inhibitors, such as bertilimumab;

Extracellular matrix protein modulators, such as CNX-024;

Fatty acid synthase inhibitors, such as TVB-2640;

Fibroblast growth factor 19 (rhFGF19)/cytochrome P450 (CYP)7A1 inhibitors, such as NGM-282;

Fibroblast growth factor 21(FGF-21) ligand, such as BMS-986171, BMS-986036;

Fibroblast growth factor 21(FGF-21)/glucagon like peptide 1 (GLP-1) agonists, such as YH-25723;

Galectin-3 inhibitors, such as GR-MD-02;

Glucagon-like peptide 1(GLP1R) agonists, such as AC-3174, liraglutide, semaglutide;

G-protein coupled bile acid receptor 1(TGR5) agonists, such as RDX-009, INT-777;

Heat shock protein 47 (HSP47) inhibitors, such as ND-L02-s0201;

HMG CoA reductase inhibitors, such as atorvastatin, fluvastatin, pitavastatin, pravastatin, rosuvastatin, and simvastatin;

IL-10 agonists, such as peg-ilodecakin;

Ileal sodium bile acid cotransporter inhibitors, such as A-4250, volixibat potassium ethanolate hydrate (SHP-262), and GSK2330672;

Insulin sensitizers, such as, KBP-042, MSDC-0602K, Px-102, RG-125 (AZD4076), and VVP-100X;

beta Klotho (KLB)-FGF1c agonist, such as NGM-313;

ketohexokinase inhibitors such as PF-06835919;

5-Lipoxygenase inhibitors, such as tipelukast (MN-001);

Lipoprotein lipase inhibitors, such as CAT-2003;

LPL gene stimulators, such as alipogene tiparvovec;

Liver X receptor (LXR) modulators, such as PX-L603, PX-L493, BMS-852927, T-0901317, GW-3965, and SR-9238;

Lysophosphatidate-1 receptor antagonists, such as BMT-053011, UD-009. AR-479, ITMN-10534, BMS-986020, and KI-16198;

Lysyl oxidase homolog 2 inhibitors, such as simtuzumab;

Semicarbazide-Sensitive Amine Oxidase/Vascular Adhesion Protein-1 (SSAO/VAP-1) Inhibitors, such as PXS-4728A;

Methionine aminopeptidase-2 inhibitors, such as ZGN-839;

Methyl CpG binding protein 2 modulators, such as mercaptamine;

Mitochondrial uncouplers, such as 2,4-dinitrophenol or nitazoxanide;

Myelin basic protein stimulators, such as olesoxime;

NADPH oxidase ¼ inhibitors, such as GKT-831;

Nicotinic acid receptor 1 agonists, such as ARI-3037MO;

NACHT LRR PYD domain protein 3 (NLRP3) inhibitors, such as KDDF-201406-03, and NBC-6;

Nuclear receptor modulators, such as DUR-928;

P2Y13 purinoceptor stimulators, such as CER-209;

PDE 3/4 inhibitors, such as tipelukast (MN-001);

PDE 5 inhibitors, such as sildenafil;

PDGF receptor beta modulators, such as BOT-191, BOT-509;

PPAR agonists, such as elafibranor (GFT-505), MBX-8025, deuterated pioglitazone R-enantiomer, pioglitazone, DRX-065, saroglitazar, and IVA-337;

Protease-activated receptor-2 antagonists, such as PZ-235;

Protein kinase modulators, such as CNX-014;

Rho associated protein kinase (ROCK) inhibitors, such as KD-025;

Sodium glucose transporter-2(SGLT2) inhibitors, such as ipragliflozin, remogliflozin etabonate, ertugliflozin, dapagliflozin, and sotagliflozin;

SREBP transcription factor inhibitors, such as CAT-2003 and MDV-4463;

Stearoyl CoA desaturase-1 inhibitors, such as aramchol;

Thyroid hormone receptor beta agonists, such as MGL-3196, MGL-3745, VK-2809;

TLR-4 antagonists, such as JKB-121;

Tyrosine kinase receptor modulators, such as CNX-025;

GPCR modulators, such as CNX-023; and

Nuclear hormone receptor modulators, such as Px-102.

In certain specific embodiments, the one or more additional therapeutic agents are selected from A-4250, AC-3174, acetylsalicylic acid, AK-20, AKN-083, alipogene tiparvovec, aramchol, ARI-3037M0, ASP-8232, atorvastatin, bertilimumab, Betaine anhydrous, BAR-704, BI-1467335, BMS-986036, BMS-986171, BMT-053011, BOT-191, BTT-1023, BWD-100, BWL-200, CAT-2003, cenicriviroc, CER-209, CF-102, CGS21680, CNX-014, CNX-023, CNX-024, CNX-025, cobiprostone, colesevelam, dapagliflozin, 16-dehydro-pregnenolone, deuterated pioglitazone R-enantiomer, 2,4-dinitrophenol, DRX-065, DS-102, DUR-928, EDP-305, elafibranor (GFT-505), emricasan, enalapril, EP-024297, ertugliflozin, evogliptin, EYP-001, F-351, fexaramine, GKT-831, GNF-5120, GR-MD-02, hydrochlorothiazide, icosapent ethyl ester, IMM-124-E, INT-767, IONIS-DGAT2Rx, INV-33, ipragliflozin, Irbesarta, propagermanium, IVA-337, JKB-121, KB-GE-001, KBP-042, KD-025, M790, M780, M450, metformin, sildenafil, LC-280126, linagliptin, liraglutide, LJN-452, LMB-763, MBX-8025, MDV-4463, mercaptamine, MET-409, MGL-3196, MGL-3745, MSDC-0602K, namacizumab, NC-101, ND-L02-s0201, NFX-21, NGM-282, NGM-313, NGM-386, NGM-395, NTX-023-1, norursodeoxycholic acid, O-304, obeticholic acid, 25HC3S, olesoxime, PAT-505, PAT-048, peg-ilodecakin, pioglitazone, pirfenidone, PRI-724, PX20606, Px-102, PX-L603, PX-L493, PXS-4728A, PZ-235, RDX-009, RDX-023, remogliflozin etabonate, repurposed tricaprilin, RG-125 (AZD4076), saroglitazar, semaglutide, simtuzumab, SIPI-7623, solithromycin, sotagliflozin, statins (atorvastatin, fluvastatin, pitavastatin, pravastatin, rosuvastatin, simvastatin), TCM-606F, TERN-101, TEV-45478, tipelukast (MN-001), TLY-012, tropifexor, TRX-318, TVB-2640, UD-009, ursodeoxycholic acid, VBY-376, VBY-825, VK-2809, vismodegib, volixibat potassium ethanolate hydrate (SHP-626), VVP-100X, WAV-301, WNT-974, and ZGN-839

In some embodiments, the one or more therapeutic agent is an ACC inhibitor described in WO2013/071169. In some embodiments, the one or more therapeutic agent is an ASK1 inhibitor described in WO2013/112741. In some embodiments, the one or more therapeutic agent is an FXR agonist such as the one described in WO2013/007387. In particular embodiments, the two therapeutic agents are an ASK1 and an ACC inhibitor. In particular embodiments, the therapeutic agents are an FXR agonist and an ASK1 inhibitor. In still other embodiments, the two therapeutic agents are an FXR agonist and an ACC inhibitor. In yet another embodiment, three therapeutic agents are used: an ASK1 inhibitor, and ACC inhibitor, and an FXR agonist.

### Pharmaceutical Compositions and Modes of Administration

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Evaluation of SOMAscan as a Discovery Platform to Identify Non-Invasive Protein Biomarkers in NASH Patients Treated with Selonsertib

This example employed SOMAscan to identify candidate protein biomarkers for diagnosis and disease monitoring in F2-F3 NASH subjects. SOMAscan is a proteomic biomarker discovery platform and has been used to identify disease-associated protein biomarkers in blood and other biological fluids.

### Method:

Seventy-two subjects with nonalcoholic steatohepatitis (NASH) (NAS ≥5) and F2-3 fibrosis were treated with selonsertib (SEL, an inhibitor of apoptosis signal-regulating kinase 1 (ASK1)) 6 mg or 18 mg orally QD alone or in combination with simtuzumab (SIM, 125 mg SQ weekly) or SIM alone for 24 weeks. Baseline and week 24 serum samples from these study subjects together with additional F0, F1 and F4 baseline samples were tested with SOMAscan (SOMAlogic, Inc., Boulder, Colo.). Associations of 1300 proteins with fibrosis stages and NAS components at baseline and after 24 weeks were examined.

### Results:

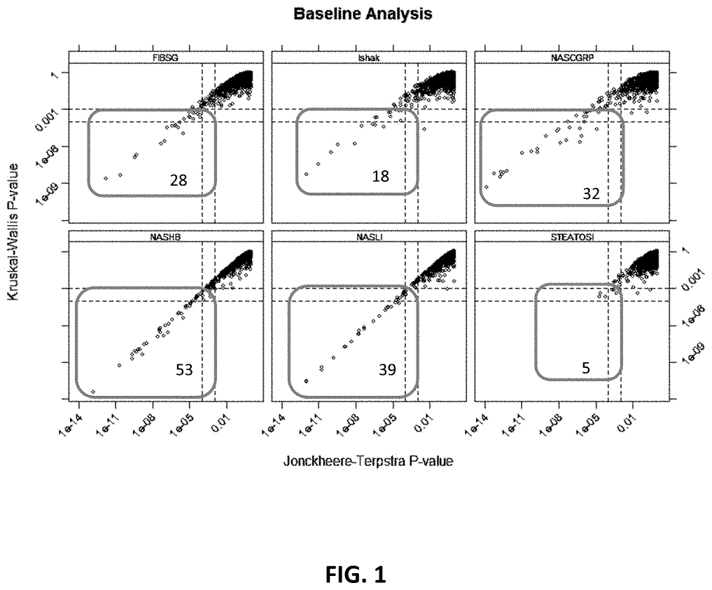

Univariate analysis identified 28 proteins that are significantly (p<0.001 for both Kruskal-Wallis test and Jonckheere-Terpstra test, only Kruskal-Wallis test p values are shown in the tables below; representative protein markers for CRN fibrosis stages are shown in FIG. 1) associated with CRN fibrosis stage, 18 proteins that are significantly associated with Ishak fibrosis stage, 34 proteins significantly associated with NAS score, 7 proteins that are significantly associated with steatosis, 39 proteins that are significantly associated with lobular inflammation, and 53 proteins that are significantly associated with hepatic ballooning at baseline. Among these proteins, several markers (ACY1, HSP90a/b, and Integrin a1b1) are associated with fibrosis stage, steatosis, lobular inflammation and hepatic ballooning. The univariate results are presented in Table 1A-F below (the protein expression levels are shown in Relative Fluorescent Unit (RFU), which is the readout from the instrument).

### Tables 1A-F: Protein Markers for Diagnosis

TABLE 1A
Protein Markers for CRN Fibrosis Stages (CRN)

| | | | CRN | CRN | CRN | CRN | CRN |
|---|---|---|---|---|---|---|---|
| | | | fibrosis | fibrosis | fibrosis | fibrosis | fibrosis |
| Marker Name | UniProt | p value | stage 0 | stage 1 | stage 2 | stage 3 | stage 4 |
| Collectin | Q9BWP8 | 2.21E-09 | 5987.2 | 4903.3 | 8174.95 | 11267.55 | 15604.5 |
| Kidney 1 | | | | | | | |
| C7 | P10643 | 4.14E-09 | 2173.6 | 1930.4 | 2330.55 | 2590.2 | 3379.95 |
| IGFBP-7 | Q16270 | 1.09E-07 | 5898.4 | 6361.5 | 7167.65 | 8593.2 | 9992.25 |
| MMP-7 | P09237 | 2.21E-07 | 1220.8 | 1321.9 | 1577.3 | 2013.65 | 2684.1 |
| Spondin-1 | Q9HCB6 | 1.53E-06 | 1600.8 | 1760.5 | 1825.3 | 2131.55 | 3023.9 |
| TSP2 | P35442 | 2.29E-06 | 575.9 | 879.3 | 1396.65 | 1761.05 | 1791.45 |
| sE-Selectin | P16581 | 3.18E-05 | 23557.3 | 25301.3 | 29424.7 | 36202.85 | 49398.9 |
| IL-1 sRI | P14778 | 4.52E-05 | 3858.3 | 3942.3 | 4198.55 | 5472.45 | 7327.05 |
| 6Ckine | 000585 | 4.25E-05 | 5152.7 | 4733.2 | 5371.65 | 6293.8 | 7693.5 |
| LTBP4 | Q8N2S1 | 5.35E-05 | 935.5 | 1033.7 | 1098.4 | 1210.85 | 1467 |
| MIC-1 | Q99988 | 3.48E-05 | 569.4 | 657 | 917 | 1046.05 | 1246.8 |
| XPNPEP1 | Q9NQW7 | 8.45E-05 | 2285.1 | 2688.5 | 3077.95 | 3519.55 | 4201.25 |
| IL-18 BPa | 095998 | 1.14E-05 | 6253.3 | 7763.5 | 8939.8 | 8942.1 | 12208.5 |
| SHP-2 | Q06124 | 0.00014999 | 2444.7 | 2746.1 | 3160.2 | 3676.75 | 4811.5 |
| IL-19 | Q9UHD0 | 0.00054898 | 6650.8 | 6412 | 7286.7 | 8225.55 | 9611.05 |
| ERAB | Q99714 | 0.00024364 | 915.8 | 1030.6 | 1101 | 1340.2 | 1274.2 |
| BSSP4 | Q9GZN4 | 0.00019407 | 816.3 | 900.9 | 974.95 | 1073.35 | 1316.85 |
| HSP 70 | P0DMV8 | 0.00084118 | 5665 | 6574.1 | 7380.75 | 8344.75 | 10561.55 |
| SEM6B | Q9H3T3 | 0.00013854 | 3329.4 | 4060.3 | 4452.6 | 4726.25 | 6304.8 |
| SLAF7 | Q9NQ25 | 0.00052829 | 46254.9 | 45233.4 | 48063.25 | 56140.5 | 74638.2 |
| CD48 | P09326 | 0.00075613 | 623.6 | 660 | 672.5 | 747.35 | 758.55 |
| FSTL3 | O95633 | 0.00088627 | 3254.9 | 3410 | 3497.55 | 3948.25 | 4403.1 |
| sCD163 | Q86VB7 | 0.00058386 | 2026.6 | 2190.2 | 2614.75 | 2745.6 | 3265.25 |
| NADPH-P450 | P16435 | 0.00043972 | 5941 | 6559.3 | 9070.3 | 10918.25 | 11295.15 |
| Oxidoreductase | | | | | | | |
| LIF sR | P42702 | 0.00084016 | 3449.2 | 3827 | 3873.75 | 4142.7 | 5350.85 |
| IL-8 | P10145 | 0.00084147 | 1456.2 | 1572.7 | 1836.55 | 1895.75 | 2405.2 |
| CD30 Ligand | P32971 | 0.0002216 | 2017.3 | 2341.4 | 2410.75 | 2459.35 | 2877.35 |
| SAP | P02743 | 0.00035833 | 34662 | 31095.8 | 29256.95 | 28337.7 | 23256.35 |

TABLE 1B
Protein Markers for Ishak Fibrosis Stages

| | | | Ishak | Ishak | Ishak | Ishak | Ishak | Ishak | Ishak |
|---|---|---|---|---|---|---|---|---|---|
| Marker | | | fibrosis | fibrosis | fibrosis | fibrosis | fibrosis | fibrosis | fibrosis |
| Name | UniProt | p value | stage 0 | stage 1 | stage 2 | stage 3 | stage 4 | stage 5 | stage 6 |
| Collectin | Q9BWP8 | 5.05E-09 | 4963.25 | 5586.5 | 8304.6 | 9376.5 | 13458.5 | 11252.75 | 24984.7 |
| Kidney 1 | | | | | | | | | |
| C7 | P10643 | 3.44E-08 | 2040.3 | 1972.6 | 2339.6 | 2419.1 | 2837.8 | 3313.525 | 3711.1 |
| IGFBP-7 | Q16270 | 3.41E-07 | 5739.9 | 6361.5 | 7210.5 | 7783.2 | 8979 | 8668.9 | 10130.15 |
| MMP-7 | P09237 | 1.14E-06 | 1271.35 | 1304 | 1590.9 | 1904.2 | 2313.6 | 2836.075 | 2784.5 |
| TSP2 | P35442 | 1.53E-06 | 738.85 | 859.4 | 1375.9 | 1426.8 | 2288.8 | 2220.55 | 1791.45 |
| sE-Selectin | P16581 | 4.76E-05 | 22373.85 | 25498 | 29931 | 34267.9 | 41464.2 | 40645.18 | 50563 |
| LTBP4 | Q8N251 | 6.18E-05 | 930.3 | 1059.7 | 1102.7 | 1113.8 | 1247.7 | 1466.4 | 1501 |
| MIC-1 | Q99988 | 4.79E-05 | 561.3 | 657 | 917.9 | 936.9 | 1144.1 | 1334.4 | 1236.3 |
| IL-1 sRI | P14778 | 0.000214 | 3701.65 | 3942.3 | 4265.2 | 5393.4 | 5330.4 | 7033.1 | 6845.45 |
| 6Ckine | 000585 | 0.000257 | 4942.95 | 4852 | 5341.4 | 6155.1 | 6818.5 | 7692.525 | 7354.8 |
| Spondin-1 | Q9HCB6 | 3.82E-05 | 1595.9 | 1871.4 | 1842.2 | 2112.1 | 2123.4 | 2916.7 | 3102.6 |
| XPNPEP1 | Q9NQW7 | 0.000539 | 2282.3 | 2753.5 | 3094.8 | 3447.2 | 3868 | 4279.625 | 4090.7 |
| ERAB | Q99714 | 0.000995 | 989.1 | 1030.6 | 1105.1 | 1258.2 | 1500.6 | 1498.35 | 1345.45 |
| BSSP4 | Q9GZN4 | 0.000525 | 858.6 | 896.9 | 981.2 | 1023.6 | 1187.2 | 1443.1 | 1316.85 |
| SAP | P02743 | 0.000271 | 31665.7 | 31374.3 | 29587.8 | 30274.7 | 27366.6 | 26341.85 | 20586.05 |
| IL-19 | Q9UHD0 | 0.001486 | 6639.5 | 6646.5 | 7290.4 | 7639.5 | 8622.7 | 8096 | 10594.2 |
| IL-8 | P10145 | 0.000723 | 1456 | 1584.9 | 1742.2 | 1857 | 1962.8 | 2554.575 | 2611.3 |
| SEM6B | Q9H3T3 | 0.000799 | 3305.25 | 4083 | 4488.2 | 4487.5 | 4742.2 | 6640.3 | 6304.8 |

TABLE 1C
Protein Markers for NAS Scores (NAS)

| Marker Name | UniProt | p value | NAS stages | | | | | | | |
|---|---|---|---|---|---|---|---|---|---|---|
| Aminoacylase-1 | Q03154 | 5.29E-10 | 4603.1 | 5770.9 | 9341.9 | 10238.4 | 14257.5 | 17923.1 | 23098.7 | 19241.9 |
| Integrin a1b1 | P56199, | 3.17E-09 | 781 | 926.8 | 956.1 | 1269.5 | 1309.1 | 2006.25 | 2542.2 | 1635 |
| | P05556 | | | | | | | | | |
| TSP2 | P35442 | 5.02E-09 | 603.9 | 652.1 | 901.8 | 975.3 | 1458.6 | 1773.25 | 2363.5 | 2284.4 |
| HSP 90a/b | P07900 | 5.26E-09 | 2710.5 | 3648.6 | 3606.3 | 4344.65 | 4465.1 | 5661.85 | 7165.9 | 7471.5 |
| | P08238 | | | | | | | | | |
| HSP 90b | P08238 | 7.07E-09 | 11239.4 | 14527.4 | 16297.7 | 18441.65 | 18554 | 24236.45 | 27701.9 | 29499.4 |
| NADPH-P450 | P16435 | 8.42E-09 | 4256.6 | 5687.5 | 6126.2 | 7149.85 | 8088 | 11520.7 | 12706.2 | 16688.9 |
| Oxidoreductase | | | | | | | | | | |
| KYNU | Q16719 | 9.53E-09 | 1596.4 | 1306.5 | 1749.9 | 1755.4 | 2097.9 | 2830.7 | 3690.8 | 3831.1 |
| ERAB | Q99714 | 2.59E-07 | 985.9 | 901.3 | 937.4 | 1032.15 | 1135.2 | 1434.65 | 1396.2 | 1497.4 |
| PHI | P06744 | 3.24E-07 | 338.2 | 503.6 | 472.7 | 575.3 | 724.1 | 789.65 | 755.2 | 1174.4 |
| FTCD | O95954 | 3.24E-07 | 2750.3 | 8873.4 | 8231.4 | 18059.9 | 21526.2 | 31366.9 | 34054.8 | 46087.7 |
| HSP 70 | PODMV8 | 6.89E-07 | 4723.9 | 5461.8 | 6315 | 7491.25 | 7694.2 | 8903.45 | 10700.5 | 10243.7 |
| sE-Selectin | P16581 | 1.74E-06 | 16016.8 | 20281.3 | 29931 | 28256.45 | 30675.2 | 36832.25 | 41029 | 45677.7 |
| Testican-1 | Q08629 | 2.15E-06 | 11858.9 | 9319.1 | 9409.3 | 9169.7 | 7865.6 | 7660.65 | 7854.7 | 8924.2 |
| ApoM | 095445 | 2.99E-06 | 5405.9 | 3828.2 | 4477.2 | 3809.6 | 3240.1 | 3155.35 | 3195.5 | 3026.8 |
| XPNPEP1 | Q9NQW7 | 6.28E-06 | 1941.6 | 2384.2 | 2429.7 | 2932.9 | 3328.7 | 3635.1 | 4251.6 | 4766.6 |
| HEMK2 | Q9Y5N5 | 6.63E-06 | 11355.9 | 9257 | 9087.2 | 8659.15 | 7762 | 7573.85 | 7178 | 8489.3 |
| Collectin | Q9BWP8 | 1.92E-05 | 4522.9 | 4689.7 | 7122.6 | 8226.4 | 8045.3 | 12168 | 16342.4 | 10018.1 |
| Kidney 1 | | | | | | | | | | |
| ENPP7 | Q6UWV6 | 2.20E-05 | 6355.4 | 2192 | 4459.6 | 6498.85 | 7703.1 | 6493.85 | 10944.8 | 12407.3 |
| AK1A1 | P14550 | 2.51E-05 | 1170 | 1582.5 | 1771.3 | 1691.95 | 1915.8 | 2408.75 | 3121.6 | 2642.9 |
| C1-Esterase | P05155 | 2.74E-05 | 6066.3 | 7494.9 | 8541.6 | 6895.8 | 6346.3 | 5897.9 | 6176.7 | 5234.7 |
| Inhibitor | | | | | | | | | | |
| HSP70 protein 8 | P11142 | 3.25E-05 | 2452.1 | 2911.8 | 2661.3 | 2979.2 | 3134.4 | 3255.35 | 3466.1 | 4060.1 |
| Cytochrome c | P99999 | 9.34E-05 | 1771.5 | 1339.2 | 1412.4 | 1316.25 | 1244.5 | 1194 | 1205.1 | 1314.5 |
| C7 | P10643 | 0.000107 | 1930.4 | 2111.3 | 2267.9 | 2153.35 | 2368.8 | 2643.25 | 3093.5 | 2804.6 |
| Cathepsin D | P07339 | 0.00025 | 867.8 | 731 | 955.1 | 893.8 | 1054.5 | 1200.2 | 1402.9 | 1037.7 |
| CD30 Ligand | P32971 | 0.000279 | 2209.5 | 2067.4 | 2239 | 2420.4 | 2458 | 2524.5 | 2688.7 | 2568.3 |
| YES | P07947 | 0.000323 | 1122.1 | 1187 | 1331.5 | 1359.2 | 1426.9 | 1522.05 | 1684.5 | 1692.5 |
| PIGR | P01833 | 0.000487 | 3151.5 | 2740.5 | 4116.2 | 3579.5 | 4070.7 | 4843.85 | 4827.2 | 6861.9 |
| QORL1 | 095825 | 0.000554 | 2484.4 | 2828.5 | 3099.3 | 3354.25 | 3708.7 | 3837.05 | 2982.6 | 3195.1 |
| AMPM 2 | P50579 | 0.000565 | 9067.1 | 10689.6 | 14417.8 | 13313.45 | 13071.7 | 16369.35 | 16596.3 | 20628.8 |
| IL-19 | Q9UHD0 | 0.000619 | 5871.2 | 5731.1 | 7359.9 | 6945.55 | 7826.4 | 7867.15 | 10139.2 | 7308 |
| BMP-1 | P13497 | 0.000632 | 843.3 | 886.9 | 1257.2 | 1245.3 | 1141.3 | 1153.3 | 1272.7 | 1329.6 |
| CAMK2D | Q13557 | 0.000684 | 2680.4 | 2866.5 | 2892.3 | 3264.25 | 3420.5 | 3633.15 | 3776.1 | 4058.2 |
| sCD163 | Q86VB7 | 0.000766 | 2429.2 | 1943.2 | 2362.6 | 2258.15 | 2717.5 | 2782.95 | 2917.8 | 3033 |
| M-CSF R | P07333 | 0.000838 | 321.9 | 246.8 | 294.6 | 320.35 | 361.3 | 356.35 | 486.9 | 367 |

TABLE 1D
Protein Markers for Steatosis

| | | | Steatosis | Steatosis | Steatosis | Steatosis |
|---|---|---|---|---|---|---|
| Marker Name | UniProt | p value | stage 0 | stage 1 | stage 2 | stage 3 |
| HSP 90b | P08238 | 0.000218 | 14145.9 | 19107.9 | 22238.45 | 26074 |
| HSP 90a/b | P07900 | 0.000221 | 3373.4 | 4670.85 | 5334.6 | 6426.6 |
| | P08238 | | | | | |
| HSP 70 | P0DMV8 | 0.000354 | 5647.5 | 8043.75 | 8569.6 | 7897.6 |
| CAMK2D | Q13557 | 0.000453 | 2680.4 | 3298.3 | 3753.95 | 3728.9 |
| Integrin a1b1 | P56199, | 0.000688 | 866.5 | 1478.55 | 1602.65 | 1464.8 |
| | P05556 | | | | | |
| Aminoacylase-1 | Q03154 | 0.000779 | 6993 | 13331 | 15128.35 | 18432.1 |

TABLE 1E
Protein Markers for Lobular Inflammation (LI)

| | | | Lobular | Lobular | Lobular |
|---|---|---|---|---|---|
| | | | inflammation | inflammation | inflammation |
| Marker Name | UniProt | p value | stage 0 | stage 1 | stage 2 |
| NADPH-P450 | P16435 | 3.03E−11 | 5303.25 | 7040 | 12303.55 |
| Oxidoreductase | | | | | |
| Aminoacylase-1 | Q03154 | 2.63E−11 | 6042.6 | 11085.1 | 19724.4 |
| Integrin a1b1 | P56199, | 2.75E−11 | 869.1 | 1233.25 | 2041.7 |
| | P05556 | | | | |
| KYNU | Q16719 | 2.69E−10 | 1400.15 | 1837.35 | 3042.45 |
| HSP 90b | P08238 | 3.87E−10 | 14336.65 | 17853.9 | 25489.65 |
| HSP 90a/b | P07900 | 2.36E−09 | 3646.55 | 4192.55 | 6228.25 |
| | P08238 | | | | |
| FTCD | O95954 | 1.62E−08 | 5909.3 | 14891.15 | 32845.3 |
| ERAB | Q99714 | 2.01E−08 | 911.15 | 1083 | 1386.65 |
| HSP 70 | P0DMV8 | 4.56E−08 | 5751.85 | 7117.95 | 9299.45 |
| XPNPEP1 | Q9NQW7 | 2.09E−07 | 2446.55 | 2814.9 | 3918.35 |
| PHI | P06744 | 1.73E-07 | 472.1 | 592.95 | 861.4 |
| Collectin | Q9BWP8 | 3.68E−07 | 4675.05 | 7928.35 | 12412.75 |
| Kidney 1 | | | | | |
| sE-Selectin | P16581 | 5.63E−07 | 18974.2 | 29030.55 | 38522.55 |
| TSP2 | P35442 | 7.19E−07 | 647.3 | 1112.8 | 1953.2 |
| AK1A1 | P14550 | 1.95E−06 | 1572.15 | 1784.1 | 2571.85 |
| dopa | P20711 | 5.65E−06 | 335.95 | 341.85 | 415.3 |
| decarboxylase | | | | | |
| HTRA2 | O43464 | 1.73E−05 | 4398.7 | 4940.3 | 5524.3 |
| YES | P07947 | 2.33E−05 | 1178.4 | 1335.15 | 1612.3 |
| ApoM | O95445 | 2.90E−05 | 4449.85 | 3773.2 | 3155.35 |
| AMPM2 | P50579 | 3.23E−05 | 11354.55 | 13312.3 | 16787.55 |
| IGFBP-7 | Q16270 | 3.56E−05 | 6824.55 | 7146.1 | 8682.05 |
| C1-Esterase | P05155 | 7.05E−05 | 7848.45 | 6795.4 | 5911.95 |
| Inhibitor | | | | | |
| C7 | P10643 | 0.000127 | 2111.25 | 2284.6 | 2665.55 |
| PIK3CA/PIK3R1 | P42336 | 0.000174 | 777.1 | 826.75 | 922 |
| | P27986 | | | | |
| HSP70 protein 8 | P11142 | 0.000131 | 2903.05 | 2959.65 | 3370.4 |
| PIGR | P01833 | 7.61E−05 | 2946 | 3806.8 | 4801.1 |
| CD30 Ligand | P32971 | 5.74E−05 | 2126.05 | 2395.3 | 2611.6 |
| IGFBP-5 | P24593 | 0.000147 | 1792.4 | 1881.85 | 1671.25 |
| NAGK | Q9UJ70 | 0.000234 | 816.05 | 931.05 | 1051.2 |
| PLXB2 | O15031 | 0.000303 | 2789.9 | 3417.55 | 3817.1 |
| IL-19 | Q9UHD0 | 0.000307 | 5868.8 | 7329.4 | 8338.8 |
| sCD163 | Q86VB7 | 0.000396 | 2001.7 | 2413.4 | 2846.25 |
| MDHC | P40925 | 0.0006 | 10621.8 | 12866.5 | 15239.55 |
| PLXC1 | O60486 | 0.000462 | 801.4 | 863.9 | 973.3 |
| IL-1 sRII | P27930 | 0.000441 | 7385.85 | 8902.45 | 10079.9 |
| HEMK2 | Q9Y5N5 | 0.000244 | 9389.7 | 8514.75 | 7863.05 |
| Testican-1 | Q08629 | 0.000375 | 9543.6 | 8546.45 | 7860.5 |
| NSE | P09104 | 0.000543 | 1103.55 | 1001.9 | 929 |
| Cathepsin D | P07339 | 0.00084 | 806.9 | 959.85 | 1153.2 |

TABLE 1F
Protein Markers for Hepatic Ballooning (HB)

| | | | Hepatic | Hepatic | Hepatic |
|---|---|---|---|---|---|
| | | | ballooning | ballooning | ballooning |
| Marker Name | UniProt | p value | stage 0 | stage 1 | stage 2 |
| TSP2 | P35442 | 3.86E−12 | 767.6 | 1105.2 | 1973.2 |
| Aminoacylase-1 | Q03154 | 5.85E−10 | 9112.9 | 11498.3 | 17923.1 |
| ERAB | Q99714 | 1.85E−09 | 985.9 | 1062.8 | 1364.45 |
| Integrin a1b1 | P56199, P05556 | 4.79E−09 | 949.1 | 1291.2 | 2006.25 |
| HSP 90a/b | P07900 | 7.19E−09 | 3648.6 | 4465.1 | 5581.3 |
| | P08238 | | | | |
| NADPH-P450 | P16435 | 7.22E−09 | 5941 | 8104.5 | 11520.7 |
| Oxidoreductase | | | | | |
| HSP 90b | P08238 | 1.28E−08 | 15659.5 | 18730.6 | 23601.1 |
| C7 | P10643 | 1.07E−08 | 2100.3 | 2173.8 | 2706.1 |
| Collectin Kidney 1 | Q9BWP8 | 3.82E−09 | 5987.2 | 8714.3 | 11197.75 |
| KYNU | Q16719 | 2.94E−08 | 1596.4 | 1796.6 | 2883.4 |
| PHI | P06744 | 3.31E−08 | 472.7 | 577.1 | 789.65 |
| XPNPEP1 | Q9NQW7 | 1.26E−07 | 2404.7 | 3127.6 | 3706.4 |
| Cathepsin D | P07339 | 5.34E−07 | 778.3 | 924.5 | 1214.15 |
| FTCD | O95954 | 3.47E−07 | 8873.4 | 16470.2 | 30715.2 |
| HSP 70 | P0DMV8 | 6.08E−07 | 6129.5 | 7057.1 | 9118.35 |
| AK1A1 | P14550 | 2.99E−07 | 1582.5 | 1757.3 | 2286.8 |
| sCD163 | Q86VB7 | 1.68E−07 | 1966.1 | 2472.6 | 2863.15 |
| HEMK2 | Q9Y5N5 | 8.82E−07 | 9257 | 8618.6 | 7700.3 |
| ApoM | O95445 | 2.58E−07 | 4477.2 | 3640 | 3174.15 |
| sE-Selectin | P16581 | 1.23E−06 | 25015.8 | 29949.5 | 36832.25 |
| Testican-1 | Q08629 | 2.69E−06 | 9409.3 | 8911.2 | 7820.55 |
| HSP70 protein 8 | P11142 | 9.96E−06 | 2844.1 | 2991.3 | 3343 |
| Cytochrome c | P99999 | 9.56E−06 | 1369.4 | 1317.6 | 1205.7 |
| SHP-2 | Q06124 | 8.69E−06 | 2489.8 | 2969.1 | 3735.45 |
| CD30 Ligand | P32971 | 3.86E−06 | 2123.2 | 2429.3 | 2611.6 |
| C1-Esterase Inhibitor | P05155 | 2.92E−05 | 7494.9 | 6866.2 | 6074.35 |
| YES | P07947 | 3.45E−05 | 1219.3 | 1348.4 | 1569 |
| PIGR | P01833 | 3.58E−05 | 3612.4 | 3694.2 | 4973.5 |
| PLXC1 | O60486 | 3.54E−05 | 801.1 | 898.8 | 964.3 |
| IL-1 sRI | P14778 | 6.90E−05 | 3581.3 | 4558.4 | 5651.75 |
| M-CSF R | P07333 | 7.07E−05 | 283.4 | 324.8 | 371.55 |
| CAMK2D | Q13557 | 5.51E−05 | 2931.9 | 3127.5 | 3711.65 |
| FABPL | P07148 | 9.97E−05 | 4770.5 | 5401.5 | 6909.6 |
| HSP 60 | P10809 | 0.000107 | 1757 | 1693.8 | 1972.3 |
| IGFBP-7 | Q16270 | 0.000172 | 6612.2 | 7445 | 8525.65 |
| Gelsolin | P06396 | 0.000172 | 553.7 | 520.1 | 464.05 |
| CD48 | P09326 | 0.000204 | 657.9 | 688.7 | 753.05 |
| PSA | P07288 | 0.000237 | 1664.7 | 1435.4 | 1245 |
| IGFBP-5 | P24593 | 0.000229 | 1999.2 | 1787 | 1700.5 |
| MMP-7 | P09237 | 0.00022 | 1365.3 | 1604.2 | 2048.9 |
| IL-19 | Q9UHD0 | 0.000303 | 6650.8 | 7034.5 | 8107.95 |
| AMPM2 | P50579 | 0.000336 | 12818.4 | 13425.9 | 16362.15 |
| Nectin-like protein 2 | Q9BY67 | 0.000463 | 1700.5 | 1818.9 | 1997.55 |
| Glutamate | Q96KP4 | 0.000262 | 4745.6 | 4343 | 4036.1 |
| carboxypeptidase | | | | | |
| CATZ | Q9UBR2 | 0.000709 | 4278.7 | 4655.3 | 5183.05 |
| ENPP7 | Q6UWV6 | 0.000121 | 3489.3 | 6355.8 | 7882.05 |
| PLXB2 | O15031 | 0.000752 | 3023.5 | 3310.8 | 3786 |
| IL-18 BPa | O95998 | 0.000771 | 7737.2 | 8266.1 | 9431.35 |
| dopa decarboxylase | P20711 | 0.000963 | 337.7 | 366.2 | 404.55 |
| STAT1 | P42224 | 0.000756 | 713.3 | 800.6 | 918.45 |
| FSTL3 | O95633 | 0.000891 | 3377.9 | 3393.1 | 4021.05 |
| IL-18 Ra | Q13478 | 0.00034 | 1010.8 | 965.4 | 1109.1 |
| IL-1 R AcP | Q9NPH3 | 0.00022 | 16533.8 | 13461.7 | 12779.85 |

The markers in Tables 1A-F were further manually filtered with their activities and other information and the filtered lists are presented in Tables 2A-F below.

### Tables 2A-F: Protein Markers for Diagnosis, Filtered

TABLE 2A
Protein Markers for CRN Fibrosis Stages, Filtered

| Marker Name | UniProt |
|---|---|
| MMP-7 | P09237 |
| Spondin-1 | Q9HCB6 |
| sE-Selectin | P16581 |
| IL-1 sRI | P14778 |
| 6Ckine | O00585 |
| LTBP4 | Q8N2S1 |
| MIC-1 | Q99988 |
| XPNPEP1 | Q9NQW7 |
| IL-18 BPa | O95998 |
| SHP-2 | Q06124 |
| IL-19 | Q9UHD0 |
| ERAB | Q99714 |
| BSSP4 | Q9GZN4 |
| HSP 70 | P0DMV8 |
| SEM6B | Q9H3T3 |
| SLAF7 | Q9NQ25 |
| CD48 | P09326 |
| FSTL3 | O95633 |
| sCD163 | Q86VB7 |
| LIF sR | P42702 |
| IL-8 | P10145 |
| CD30 Ligand | P32971 |
| SAP | P02743 |

TABLE 2B
Protein Markers for Ishak Fibrosis Stages, Filtered

| Marker Name | UniProt |
|---|---|
| MMP-7 | P09237 |
| sE-Selectin | P16581 |
| LTBP4 | Q8N2S1 |
| MIC-1 | Q99988 |
| IL-1 sRI | P14778 |
| 6Ckine | O00585 |
| Spondin-1 | Q9HCB6 |
| XPNPEP1 | Q9NQW7 |
| ERAB | Q99714 |
| BSSP4 | Q9GZN4 |
| SAP | P02743 |
| IL-19 | Q9UHD0 |
| IL-8 | P10145 |
| SEM6B | Q9H3T3 |

TABLE 2C
Protein Markers for NAS Scores, Filtered

| Marker Name | UniProt |
|---|---|
| HSP 90b | P08238 |
| ERAB | Q99714 |
| PHI | P06744 |
| FTCD | O95954 |
| HSP 70 | P0DMV8 |
| sE-Selectin | P16581 |
| Testican-1 | Q08629 |
| ApoM | O95445 |
| XPNPEP1 | Q9NQW7 |
| HEMK2 | Q9Y5N5 |
| ENPP7 | Q6UWV6 |
| AK1A1 | P14550 |
| C1-Esterase Inhibitor | P05155 |
| HSP70 protein 8 | P11142 |
| Cytochrome c | P99999 |
| Cathepsin D | P07339 |
| CD30 Ligand | P32971 |
| YES | P07947 |
| PIGR | P01833 |
| QORL1 | O95825 |
| AMPM2 | P50579 |
| IL-19 | Q9UHD0 |
| BMP-1 | P13497 |
| CAMK2D | Q13557 |
| sCD163 | Q86VB7 |

TABLE 2D
Protein Markers for Steatosis, Filtered

| Marker Name | UniProt |
|---|---|
| HSP 70 | P0DMV8 |
| CAMK2D | Q13557 |

TABLE 2E
Protein Markers for Lobular Inflammation, Filtered

| Marker Name | UniProt |
|---|---|
| HSP 90b | P08238 |
| FTCD | O95954 |
| ERAB | Q99714 |
| HSP 70 | P0DMV8 |
| XPNPEP1 | Q9NQW7 |
| PHI | P06744 |
| sE-Selectin | P16581 |
| AK1A1 | P14550 |
| HTRA2 | O43464 |
| YES | P07947 |
| ApoM | O95445 |
| AMPM2 | P50579 |
| C1-Esterase Inhibitor | P05155 |
| HSP70 protein 8 | P11142 |
| PIGR | P01833 |
| CD30 Ligand | P32971 |
| IGFBP-5 | P24593 |
| PLXB2 | O15031 |
| IL-19 | Q9UHD0 |
| sCD163 | Q86VB7 |
| MDHC | P40925 |
| PLXC1 | O60486 |
| IL-1 sRII | P27930 |
| HEMK2 | Q9Y5N5 |
| Testican-1 | Q08629 |
| NSE | P09104 |
| Cathepsin D | P07339 |

TABLE 2F
Protein Markers for Hepatic Ballooning, Filtered

| Marker Name | UniProt |
|---|---|
| ERAB | Q99714 |
| PHI | P06744 |
| XPNPEP1 | Q9NQW7 |
| Cathepsin D | P07339 |
| FTCD | O95954 |
| HSP 70 | P0DMV8 |
| AK1A1 | P14550 |
| sCD163 | Q86VB7 |
| HEMK2 | Q9Y5N5 |
| ApoM | O95445 |
| sE-Selectin | P16581 |
| Testican-1 | Q08629 |
| HSP70 protein 8 | P11142 |
| Cytochrome c | P99999 |
| SHP-2 | Q06124 |
| CD30 Ligand | P32971 |
| C1-Esterase Inhibitor | P05155 |
| YES | P07947 |
| PIGR | P01833 |
| PLXC1 | O60486 |
| IL-1 sRI | P14778 |
| CAMK2D | Q13557 |
| FABPL | P07148 |
| HSP 60 | P10809 |
| Gelsolin | P06396 |
| CD48 | P09326 |
| PSA | P07288 |
| IGFBP-5 | P24593 |
| MMP-7 | P09237 |
| IL-19 | Q9UHD0 |
| AMPM2 | P50579 |
| Nectin-like protein 2 | Q9BY67 |
| Glutamate carboxypeptidase | Q96KP4 |
| CATZ | Q9UBR2 |
| ENPP7 | Q6UWV6 |
| PLXB2 | O15031 |
| IL-18 BPa | O95998 |
| STAT1 | P42224 |
| FSTL3 | O95633 |
| IL-18 Ra | Q13478 |
| IL-1 R AcP | Q9NPH3 |

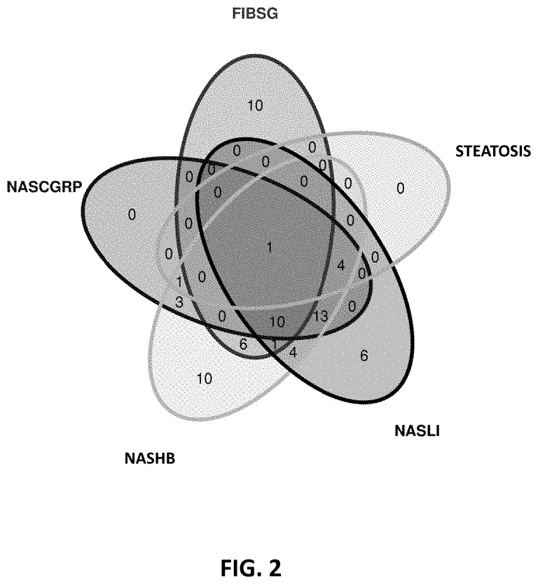

FIG. 2 shows common proteins markers are present between different diagnostic variables (FIBSG: CRN fibrosis stages; STEATOSI: steatosis; NASLI: NAS Lobular Inflammation; NASHB: NAS Hepatic Ballooning; NASCGRP: NAS score). Certain overlaps are summarized in Table A below:

TABLE A
Common Protein Markers between Groups

| Group | Markers |
|---|---|
| Unique to CRN fibrosis (10) | 6Ckine, BSSP4, IL-8, LIF sR, LTBP4, MIC-1, SAP, SEM6B, |
| | SLAF7, Spondin-1 |
| Unique to HB (10) | CATZ, FABPL, Gelsolin, Glutamate carboxypeptidase, HSP |
| | 60, IL-1 R AcP, IL-18 Ra Nectin-like protein 2, PSA, STAT1 |
| Unique to L (6) | HTRA2, IL-1 sRII, MDHC, NAGK, NSE, PIK3CA/PIK3R1 |
| Common to all 5 responsive | HSP 70 |
| variables (1) | |
| Common to CRN fibrosis/HB (6) | CD48, FSTL3, IL-1 sRI, IL-18 BPa, MMP-7, SHP-2 |
| Common to CRN fibrosis/HS/LI (1) | IGFBP-7 |
| Common to CRN | C7, CD30 Ligand, Collectin Kidney 1, ERAB, IL-19, NADPH- |
| fibrosis/NAS/HB/LI (10) | P450 Oxidoreductase, sCD163, sE-Selectin TSP2, |
| | XPNPEP1 |
| Common to NAS/HB/LI/Steatosis | Aminoacylase-1, HSP 90a/b, HSP 90b, Integrin a1b |
| (4) | |
| Common to NAS/HB (3) | Cytochrome c, ENPP7, M-CSF R |
| Common to HB/LI(4) | dopa decarboxylase, IGFBP-5, PLXB2, PLXC |
| Common to NAS/HB/Steatosis (1) | CAMK2D |
| Common to NAS/HB/LI (13) | AK1A1, AMPM2, ApoM, C1-Esterase Inhibitor, Cathepsin |
| | D, FTCD, HEMK2, HSP70 protein 8, KYNU, PHI, PmGR, |
| | Testican-1, YES |

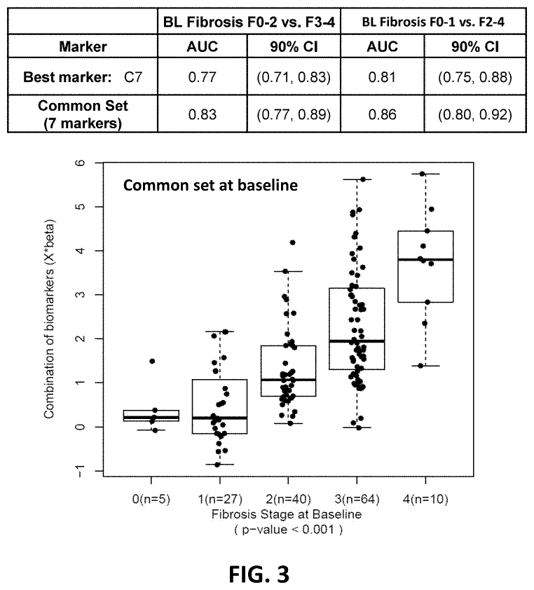

Multivariate analysis further identified a panel of 7 protein markers (C7, CL-K1, IGFBP7, Spondin 1, IL-5Ra (UniProt: Q01344), MMP-7 and TSP2) that possess good diagnostic value to classify NASH subjects with severe fibrosis (F0-1 vs F3-4; AUROC: 0.83; FIG. 3). Changes in circulating levels of the biomarkers were generally reflected in the expression of their corresponding RNAs by RNAseq of formalin-fixed paraffin-embedded (FFPE) sections of liver.

Longitudinal changes of several protein markers (KYNU, Integrin a1b1, CL-K1, C7, ACY1 and TSP2) that are associated with fibrosis stage or NAS score at baseline also significantly correlate with improvement in one or more NASH clinical features (fibrosis, steatosis and lobular inflammation). The detailed listing of protein markers for monitoring clinical improvement features are provided in Tables 3A-D below.

### Tables 3A-D: Protein Markers for Monitoring Clinical Improvements

TABLE 3A
Protein Biomarkers for Monitoring Steatosis Improvement

| | | Median percent | Median percent | |
|---|---|---|---|---|
| | | CHG at W24 in | CHG at W24 in | |
| Marker Name | UniProt | Non-improver | improver | p value |
| KYNU | Q16719 | −6.785 | −30.97 | 6.57E-05 |
| Nectin-like protein 2 | Q9BY67 | 7.925 | −7.93 | 0.000107 |
| CD30 Ligand | P32971 | 0.81 | −9.18 | 0.000215 |
| YKL-40 | P36222 | 13.28 | −14.29 | 0.000368 |
| TSP2 | P35442 | 21.215 | −25.59 | 0.000493 |
| PCSK7 | Q16549 | 7.995 | −3.57 | 0.000522 |
| IR | P06213 | 0.875 | −13.12 | 0.000576 |
| AK1A1 | P14550 | −8.95 | −28.48 | 0.000586 |
| FLRT3 | Q9NZU0 | 3.09 | −10.72 | 0.000586 |
| TIMD3 | Q8TDQ0 | 4.615 | −8.75 | 0.00062 |
| SAP | P02743 | −1.14 | 5.44 | 0.000695 |
| YES | P07947 | −0.1 | −11.71 | 0.000706 |
| Integrin a1b1 | P56199, P05556 | −5.025 | −41.26 | 0.000724 |
| CNTN2 | Q02246 | 3.91 | −6.28 | 0.000821 |
| sTie-2 | Q02763 | −0.925 | −8.34 | 0.000821 |
| Lumican | P51884 | 4.69 | −6.81 | 0.000821 |
| HSP 70 | P0DMV8 | −0.875 | −27.05 | 0.000868 |

TABLE 3B
Protein Biomarkers for Monitoring Lobular
Inflammation Improvement

| | | Median | | |
|---|---|---|---|---|
| | | percent | Median | |
| | | CHG at | percent | |
| | | W24 in | CHG at | |
| | | Non- | W24 in | |
| Marker Name | UniProt | improver | improver | p value |
| M-CSF R | P07333 | 5.72 | −12.625 | 4.58E−05 |
| ACE2 | Q9BYF1 | −2.05 | 2.605 | 0.000138 |
| FCG3B | O75015 | −1.44 | −14.29 | 0.000191 |
| IL24 | Q13007 | 2.03 | −3.985 | 0.00038 |
| ERAB | Q99714 | 2.01 | −26.665 | 0.000429 |
| Keratin 18 | P05783 | −0.96 | 4.1 | 0.000515 |
| Aminoacylase-1 | Q03154 | −11.97 | −37.08 | 0.000727 |
| PHI | P06744 | −5.37 | −30.06 | 0.000862 |
| C4 | P0C0L4, | −2.53 | 7.51 | 0.001019 |
| | P0C0L5 | | | |
| HSP 90a/b | P07900 | −4.98 | −21.04 | 0.001202 |
| | P08238 | | | |
| CHST2 | Q9Y4C5 | 1.67 | −5.34 | 0.001291 |
| Integrin a1b1 | P56199, | −5.31 | −34.885 | 0.00195 |
| | P05556 | | | |
| PIK3CA/PIK3R1 | P42336 | −2.3 | −13.095 | 0.001996 |
| | P27986 | | | |
| Cathepsin D | P07339 | 4.99 | −14.74 | 0.002711 |
| NADPH-P450 | P16435 | −8.01 | −37.395 | 0.002777 |
| Oxidoreductase | | | | |
| CBG | P08185 | −2.61 | 1.495 | 0.003557 |
| MDM2 | Q00987 | 1.43 | −3.26 | 0.004668 |
| C7 | P10643 | 5.79 | −7.535 | 0.004745 |
| CD5L | O43866 | 6.76 | −6.625 | 0.00581 |
| IL-8 | P10145 | −4.72 | −15.795 | 0.005992 |
| LYVE1 | Q9Y5Y7 | 4.61 | −4.21 | 0.00661 |
| Eotaxin | P51671 | 4.34 | 15.185 | 0.007197 |
| Albumin | P02768 | −0.65 | 13.88 | 0.007351 |
| Histone H2A.z | P0C0S5 | 10.31 | −8.545 | 0.007519 |
| KIF23 | Q02241 | −2.64 | 2.93 | 0.007519 |
| ALT | P24298 | −0.07 | −9.27 | 0.008514 |
| EPO-R | P19235 | −1.13 | 2.2 | 0.008591 |
| STX1a | Q16623 | 3.03 | −2.59 | 0.00906 |
| MED-1 | Q15648 | −2.61 | 11.545 | 0.009378 |

TABLE 3C
Protein Biomarkers for Monitoring Hepatic
Ballooning Improvement

| | | Median | | |
|---|---|---|---|---|
| | | percent | Median | |
| | | CHG at | percent | |
| | | W24 in | CHG at | |
| | | Non- | W24 in | |
| Marker Name | UniProt | improver | improver | p value |
| Protein C | P04070 | −0.09 | 9.69 | 0.000589 |
| LG3BP | Q08380 | 7.44 | −7.56 | 0.006607 |
| Cathepsin D | P07339 | 4.95 | −15.96 | 0.01054 |
| Coagulation Factor Xa | P00742 | 0.825 | 6.52 | 0.010541 |
| Thyroxine-Binding | P05543 | −1.5 | −7.58 | 0.014594 |
| Globulin | | | | |
| XEDAR | Q9HAV5 | −0.875 | 1.66 | 0.014887 |
| CBG | P08185 | −2.655 | 1.44 | 0.015188 |
| HGF | P14210 | −4.445 | −9.9 | 0.015802 |
| LTBP4 | Q8N2S1 | 2.525 | −5.93 | 0.017776 |
| Eotaxin-2 | O00175 | −0.04 | 4.07 | 0.018122 |
| B7-H2 | O75144 | −5.27 | −12.52 | 0.01921 |
| JAG1 | P78504 | 2.6 | 0.16 | 0.019963 |
| STAT3 | P40763 | −4.58 | −8.59 | 0.020743 |
| VEGF sR2 | P35968 | −1.275 | 4.03 | 0.020744 |
| Laminin | P25391, | 2.15 | −8.68 | 0.021551 |
| | P07942, | | | |
| | P11047 | | | |
| Topoisomerase I | P11387 | 6.47 | −13.48 | 0.021551 |
| IGFBP-5 | P24593 | −0.695 | 6.44 | 0.023245 |
| Heparin cofactor II | P05546 | −1.345 | 3.88 | 0.024135 |
| Osteopontin | P10451 | −1.54 | 1.79 | 0.024585 |
| BAFF | Q9Y275 | 2.16 | −7.94 | 0.026002 |
| IL-13 Ra1 | P78552 | −0.565 | 5.91 | 0.026002 |
| PH | P01298 | 6.14 | −3.88 | 0.026004 |
| EphA1 | P21709 | 1.83 | 8.65 | 0.026984 |
| HINT1 | P49773 | −10.655 | −30.56 | 0.026984 |
| kallikrein 12 | Q9UKR0 | −1.51 | 3.44 | 0.026984 |
| M2-PK | P14618 | −3.615 | −22.55 | 0.026984 |
| Lymphotoxin b R | P36941 | −0.575 | 3.35 | 0.027996 |
| C3 | P01024 | −3.82 | −24.36 | 0.029041 |
| pTEN | P60484 | −0.705 | −5.01 | 0.029041 |
| Fucosyltransferase 3 | P21217 | 3.205 | −1.89 | 0.031232 |
| Myeloperoxidase | P05164 | −9.61 | −17.96 | 0.031232 |
| Tropomyosin 2 | P07951 | −0.98 | 4.09 | 0.032377 |
| ARMEL | Q49AH0 | 1.91 | 10.94 | 0.032379 |
| PKB beta | P31751 | −0.495 | −7.77 | 0.032379 |
| I-309 | P22362 | −2.48 | 7.66 | 0.03356 |
| GDF2 | Q9UK05 | −10.91 | −21.82 | 0.033562 |
| TrkA | P04629 | −2.235 | 5.74 | 0.033562 |
| PLXB2 | O15031 | 3.24 | −3.7 | 0.03478 |
| Proteinase-3 | P24158 | −14.585 | −26.62 | 0.034782 |
| S100A7 | P31151 | 1.06 | −1.99 | 0.03604 |
| ARGI1 | P05089 | −6.98 | −19.27 | 0.037336 |
| sCD163 | Q86VB7 | 2.88 | −4.88 | 0.037995 |
| Adrenomedullin | P35318 | 1.22 | −5.44 | 0.038672 |
| MAPK14 | Q16539 | −0.58 | −5.45 | 0.040046 |
| TMA | P07202 | −0.93 | 2.55 | 0.040048 |
| Epo | P01588 | 18.195 | 1.25 | 0.041464 |
| annexin I | P04083 | 2.89 | −12.32 | 0.042189 |
| BPI | P17213 | −20.905 | −39.76 | 0.044429 |
| Collectin Kidney 1 | Q9BWP8 | −1.64 | −13.64 | 0.044429 |
| IL-17F | Q96PD4 | −1.62 | 4.93 | 0.044429 |
| JAM-B | P57087 | −0.86 | 6.48 | 0.044429 |
| Integrin aVb5 | P06756, | 1.765 | −5.42 | 0.045976 |
| | P18084 | | | |
| MP2K2 | P36507 | 1.985 | 10.3 | 0.045976 |
| SLIK1 | Q96PX8 | −0.975 | 5.51 | 0.045976 |
| SP-D | P35247 | −7.39 | −30.86 | 0.045976 |
| CD63 | P08962 | −1.285 | 4.6 | 0.048379 |
| KEAP1 | Q14145 | −0.79 | 5.74 | 0.049202 |
| calgranulin B | P06702 | −11.515 | −28.5 | 0.049204 |
| PSD7 | P51665 | 1.45 | −0.75 | 0.049204 |
| RNase H1 | 060930 | 0.585 | −4.06 | 0.049204 |
| GNS | P15586 | 3.03 | −6.64 | 0.049207 |

TABLE 3D
Protein Biomarkers for Monitoring CRN
Fibrosis Stage Improvement

| | | Median | | |
|---|---|---|---|---|
| | | percent | Median | |
| | | CHG at | percent | |
| | | W24 in | CHG at | |
| | | Non- | W24 in | |
| Marker Name | UniProt | improver | improver | p value |
| GITR | Q9Y5U5 | −1.84 | −7.67 | 0.00178 |
| Cathepsin H | P09668 | −3.02 | −13.86 | 0.0038 |
| DcR3 | O95407 | −2.01 | 1.1 | 0.005199 |
| LAG-1 | Q8NHW4 | −4.56 | −11.84 | 0.00619 |
| PDXK | O00764 | −2.82 | −13.7 | 0.00766 |
| RNase H1 | O60930 | −2.6 | 2.2 | 0.007865 |
| GP1BA | P07359 | −4.35 | 1.52 | 0.009824 |
| IL-1a | P01583 | −0.12 | −4.67 | 0.011627 |
| GIB | P04054 | 2.12 | −12.9 | 0.012548 |
| carbonic | P00918 | −2 | 4.59 | 0.01301 |
| anhydrase II | | | | |
| Caspase-2 | P42575 | −2.27 | 2.84 | 0.015708 |
| Cystatin-S | P01036 | −1.78 | 3.35 | 0.016421 |
| Troponin I, | P48788 | 6.48 | −1.77 | 0.018398 |
| skeletal, | | | | |
| fast twitch | | | | |
| IF4A3 | P38919 | −1.61 | 2.58 | 0.02063 |
| PSMA | Q04609 | 1.42 | −6.53 | 0.021352 |
| Dtk | Q06418 | 2.32 | −1.06 | 0.022546 |
| LY9 | Q9HBG7 | 3.6 | −6.33 | 0.022975 |
| MK13 | O15264 | −4.33 | 0.54 | 0.024616 |
| KI3L2 | P43630 | −1.21 | 1.92 | 0.024702 |
| TrkC | Q16288 | 3.16 | −4.72 | 0.026387 |
| PCI | P05154 | 12.19 | 1.79 | 0.026539 |
| TNFSF15 | O95150 | 2.31 | −2.49 | 0.026539 |
| Coagulation | P00740 | 6.7 | 0.35 | 0.0275 |
| Factor IX | | | | |
| MMP-3 | P08254 | 12.69 | 2.97 | 0.027786 |
| INGR2 | P38484 | −1 | 2.76 | 0.028756 |
| C3a | P01024 | −19.78 | 15.9 | 0.029511 |
| LRP8 | Q14114 | 1.37 | −2.81 | 0.029511 |
| 17-beta-HSD 1 | P14061 | −4.14 | 2.16 | 0.029753 |
| MP2K3 | P46734 | −3.46 | −0.97 | 0.030562 |
| CD70 | P32970 | −1.88 | 3.16 | 0.031303 |
| CPNE1 | Q99829 | −4.36 | 2.87 | 0.031645 |
| NEUREGULIN-1 | Q02297 | −0.69 | 6.86 | 0.031645 |
| PCSK9 | Q8NBP7 | −2.85 | 8.12 | 0.031645 |
| pTEN | P60484 | −4.94 | 2.32 | 0.031645 |
| K-ras | P01116 | 0.27 | −3.79 | 0.033907 |
| ATS15 | Q8TE58 | −0.15 | 4.2 | 0.036304 |
| Ephrin-A5 | P52803 | 5.39 | −0.13 | 0.036304 |
| Endoglin | P17813 | 1.62 | −3.86 | 0.037555 |
| AFP | P02771 | −0.39 | 1.07 | 0.037603 |
| FTCD | O95954 | −6.71 | −42.29 | 0.037603 |
| Granzyme B | P10144 | −1.2 | 2.74 | 0.038842 |
| Activin RIB | P36896 | −2.91 | 0.64 | 0.038856 |
| TGF-b3 | P10600 | −0.94 | 2.97 | 0.040146 |
| DSC3 | Q14574 | 1.23 | 6.37 | 0.040165 |
| WFKN2 | Q8TEU8 | 0.47 | −2.98 | 0.042148 |
| c-Myc | P01106 | −2.91 | 0.18 | 0.043526 |
| b-Endorphin | P01189 | -0.56 | 2.33 | 0.043529 |
| MICA | Q29983 | 1.73 | −5.58 | 0.044362 |
| ART | Q00253 | 9.05 | −1.82 | 0.04584 |
| CD30 Ligand | P32971 | −0.44 | −4.89 | 0.047903 |
| GV | P39877 | 0.22 | 3.21 | 0.048664 |
| CLC7A | Q9BXN2 | −3.19 | 3.23 | 0.048918 |
| IL-17D | Q8TAD2 | 0.85 | −1.33 | 0.048918 |
| KYNU | Q16719 | −8.6 | −26.22 | 0.048918 |
| SLAF5 | Q9UIB8 | −4.04 | −0.59 | 0.048918 |
| SSRP1 | Q08945 | −1.51 | −0.48 | 0.049438 |

The markers were further manually filtered with their activities and other information and the filtered lists are presented in Tables 4A-D below.

### Tables 4A-D: Protein Markers for Monitoring Clinical Improvements, Filtered

TABLE 4A
Protein Biomarkers for Monitoring Steatosis
Improvement, Filtered

| Marker Name | UniProt |
|---|---|
| Nectin-like protein 2 | Q9BY67 |
| CD30 Ligand | P32971 |
| YKL-40 | P36222 |
| TSP2 | P35442 |
| PCSK7 | Q16549 |
| IR | P06213 |
| AK1A1 | P14550 |
| FLRT3 | Q9NZU0 |
| TIMD3 | Q8TDQ0 |
| SAP | P02743 |
| YES | P07947 |
| CNTN2 | Q02246 |
| sTie-2 | Q02763 |
| Lumican | P51884 |
| HSP 70 | P0DMV8 |

TABLE 4B
Protein Biomarkers for Monitoring Lobular
Inflammation Improvement, Filtered

| Marker Name | UniProt |
|---|---|
| ACE2 | Q9BYF1 |
| IL24 | Q13007 |
| ERAB | Q99714 |
| Keratin 18 | P05783 |
| PHI | P06744 |
| C4 | P0C0L4, |
| | P0C0L5 |
| CHST2 | Q9Y4C5 |
| Cathepsin D | P07339 |
| CBG | P08185 |
| MDM2 | Q00987 |
| CD5L | O43866 |
| IL-8 | P10145 |
| LYVE1 | Q9Y5Y7 |
| Eotaxin | P51671 |
| Albumin | P02768 |
| Histone H2A.z | P0C0S5 |
| KIF23 | Q02241 |
| ALT | P24298 |
| EPO-R | P19235 |
| STX1a | Q16623 |
| MED-1 | Q15648 |

TABLE 3C
Protein Biomarkers for Monitoring Hepatic Ballooning
Improvement, Filtered

| Marker Name | UniProt |
|---|---|
| Protein C | P04070 |
| LG3BP | Q08380 |
| Cathepsin D | P07339 |
| Coagulation Factor Xa | P00742 |
| Thyroxine-Binding | P05543 |
| Globulin | |
| XEDAR | Q9HAV5 |
| CBG | P08185 |
| HGF | P14210 |
| LTBP4 | Q8N2S1 |
| Eotaxin-2 | O00175 |
| B7-H2 | O75144 |
| JAG1 | P78504 |
| STAT3 | P40763 |
| VEGF sR2 | P35968 |
| Laminin | P25391, |
| | P07942, |
| | P11047 |
| Topoisomerase I | P11387 |
| IGFBP-5 | P24593 |
| Heparin cofactor II | P05546 |
| Osteopontin | P10451 |
| BAFF | Q9Y275 |
| IL-13 Ra1 | P78552 |
| PH | P01298 |
| EphA1 | P21709 |
| HINT1 | P49773 |
| kallikrein 12 | Q9UKR0 |
| M2-PK | P14618 |
| Lymphotoxin b R | P36941 |
| C3 | P01024 |
| pTEN | P60484 |
| Fucosyltransferase 3 | P21217 |
| Myeloperoxidase | P05164 |
| Tropomyosin 2 | P07951 |
| ARMEL | Q49AH0 |
| PKB beta | P31751 |
| 1-309 | P22362 |
| GDF2 | Q9UK05 |
| TrkA | P04629 |
| PLXB2 | O15031 |
| Proteinase-3 | P24158 |
| S100A7 | P31151 |
| ARGI1 | P05089 |
| sCD163 | Q86VB7 |
| Adrenomedullin | P35318 |
| MAPK14 | Q16539 |
| TMA | P07202 |
| Epo | P01588 |
| annexin 1 | P04083 |
| BPI | P17213 |
| IL-17F | Q96PD4 |
| JAM-B | P57087 |
| Integrin aVb5 | P06756, |
| | P18084 |
| MP2K2 | P36507 |
| SLIK1 | Q96PX8 |
| SP-D | P35247 |
| CD63 | P08962 |
| KEAP1 | Q14145 |
| calgranulin B | P06702 |
| PSD7 | P51665 |
| RNase H1 | O60930 |
| GNS | P15586 |

TABLE 4D
Protein Biomarkers for Monitoring CRN
Fibrosis Stage Improvement, Filtered

| Marker Name | UniProt |
|---|---|
| GITR | Q9Y5U5 |
| Cathepsin H | P09668 |
| DcR3 | O95407 |
| LAG-1 | Q8NHW4 |
| PDXK | O00764 |
| RNase H1 | O60930 |
| GP1BA | P07359 |
| IL-1a | P01583 |
| GIB | P04054 |
| carbonic | P00918 |
| anhydrase II | |
| Caspase-2 | P42575 |
| Cystatin-S | P01036 |
| Troponin I, | P48788 |
| skeletal, fast | |
| twitch | |
| IF4A3 | P38919 |
| PSMA | Q04609 |
| Dtk | Q06418 |
| LY9 | Q9HBG7 |
| MK13 | O15264 |
| KI3L2 | P43630 |
| TrkC | Q16288 |
| PCI | P05154 |
| TNFSF15 | O95150 |
| Coagulation | P00740 |
| Factor IX | |
| MMP-3 | P08254 |
| INGR2 | P38484 |
| C3a | P01024 |
| LRP8 | Q14114 |
| 17-beta-HSD 1 | P14061 |
| MP2K3 | P46734 |
| CD70 | P32970 |
| CPNE1 | Q99829 |
| NEUREGULIN-1 | Q02297 |
| PCSK9 | Q8NBP7 |
| pTEN | P60484 |
| K-ras | P01116 |
| ATS15 | Q8TE58 |
| Ephrin-A5 | P52803 |
| Endoglin | P17813 |
| AFP | P02771 |
| FTCD | O95954 |
| Granzyme B | P10144 |
| Activin RIB | P36896 |
| TGF-b3 | P10600 |
| DSC3 | Q14574 |
| WFKN2 | Q8TEU8 |
| c-Myc | P01106 |
| b-Endorphin | P01189 |
| MICA | Q29983 |
| ART | O00253 |
| CD30 Ligand | P32971 |
| GV | P39877 |
| CLC7A | Q9BXN2 |
| IL-17D | Q8TAD2 |
| SLAF5 | Q9UIB8 |
| SSRP1 | Q08945 |

Multivariate analysis for the monitoring markers also identifies a few groups of markers, when used collectively, possess better monitoring capabilities. These multivariate marker groups are listed in Table B below.

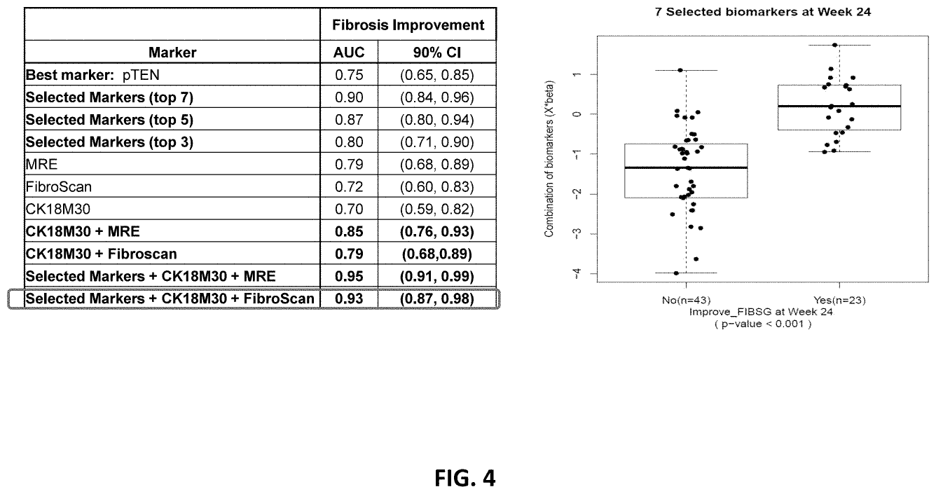

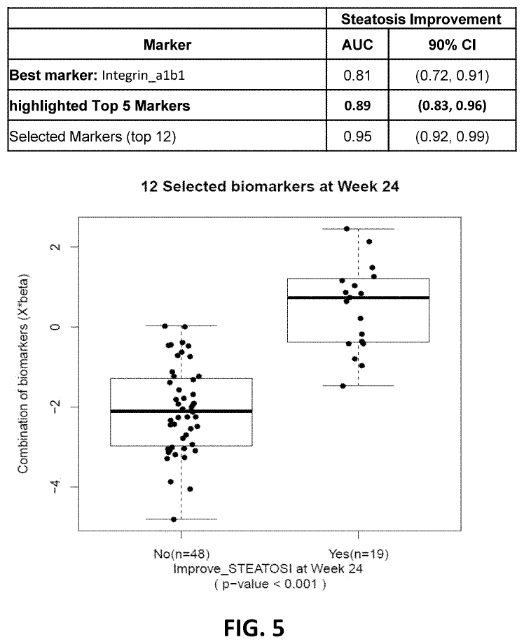

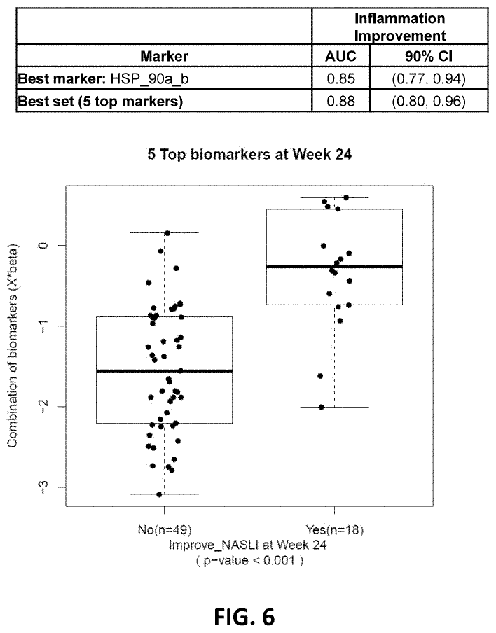

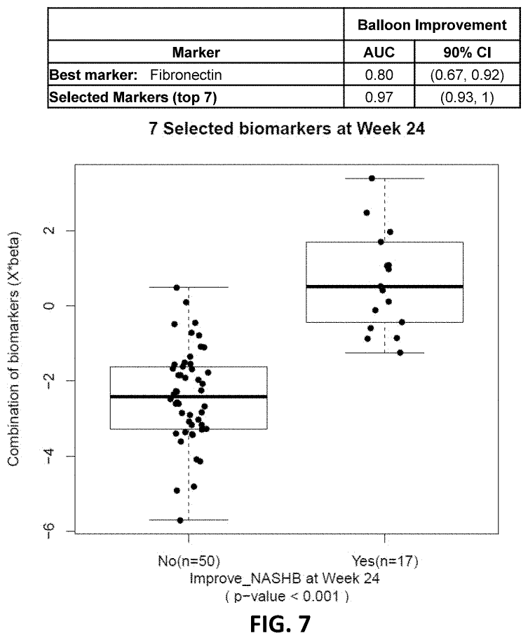

TABLE B
Multivariate Protein Markers for Treatment Monitoring

| Endpoint | Protein Markers | | FIG. |
|---|---|---|---|
| CRN Fibrosis stage | Marker | (UniProt) | FIG. 4 |
| | pTEN | (P60484) | |
| | CD70 | (P32970) | |
| | Caspase-2 | (P42575) | |
| | Cathepsin H | (P09668) | |
| | LAG-1 | (Q8NHW4) | |
| | PDXK | (O00764) | |
| | GITR | (Q9Y5U5) | |
| Steatosis | Marker | (UniProt) | FIG. 5 |
| | Integrin a1b1 | (P56199, P05556) | |
| | Nectin-like protein 2 | (Q9BY67) | |
| | PDGF Rb | (P09619) | |
| | LRP8 | (Q14114) | |
| | CD30 Ligand | (P32971) | |
| | Lumican | (P51884) | |
| | SAP | (P02743) | |
| | YKL-40 | (P36222) | |
| | sTie-2 | (Q02763) | |
| | HSP 90a/b | (P07900 P08238) | |
| | TSP2 | (P35442) | |
| | YES | (P07947) | |
| Lobular | | | |
| Inflammation | Marker | (UniProt) | FIG. 6 |
| | HSP 90a/b | (P07900 P08238) | |
| | Aminoacylase-1 | (Q03154) | |
| | FCG3B | (O75015) | |
| | M-CSF R | (P07333) | |
| | Keratin 18 | (P05783) | |
| Hepatic Ballooning | Marker | (UniProt) | FIG. 7 |
| | Fibronectin | (P02751) | |
| | Thyroxine-Binding | (P05543) | |
| | Globulin | | |
| | FGF23 | (Q9GZV9) | |
| | LG3BP | (Q08380) | |
| | Heparin cofactor II | (P05546) | |
| | Protein C | (P04070) | |
| | STAT3 | (P40763) | |

This example identifies new protein biomarker candidates for staging fibrosis, steatosis, lobular inflammation, and hepatic ballooning in NASH subjects are identified using SOMAscan. Additionally, in F2-3 NASH subjects treated with selonsertib, markers that show treatment response monitoring characteristics are also identified.

### Example 2. Serum Bile Acid Levels are Reciprocally Regulated with C4 Levels Across the Spectrum of Disease Severity in Patients with Nonalcoholic Steatohepatitis (NASH)

Serum bile acid (BA) profiles are altered in patients with NASH. Data describing associations between BA composition and fibrosis stage are limited. This example was performed to: 1) characterize circulating BAs and its intermediate, 7α-hydroxy-4-cholesten-3-one (C4), in patients across the spectrum of NASH and healthy controls; and 2) determine associations between serum BAs and fibrosis stage in NASH.

### Methods:

Fasting serum levels of 15 BAs were quantified in healthy controls (n=118), NASH F2/3 (n=72), and NASH cirrhosis (n=29) by LC-MS/MS (Agilent 1290/Sciex, Metabolon). Serum from clinical studies of healthy controls in PK studies of Compound A (a selective, non-steroidal agonist of the Farnesoid X receptor), simtuzumab in F2/3 fibrosis, and cirrhosis was used. Analysis of serum BA from cirrhosis patients occurred prior and after clinical decompensation. Serum C4 levels were measured to reflect hepatic bile acid biosynthesis. One-way ANOVA was used to assess differences in BA and C4 levels between groups. Data presented as median (IQR).

### Results:

Fasting total serum BA levels in healthy controls were 877 ng/mL (582, 1298) and increased in a step-wise fashion with progressive fibrosis: F2, 1483 (937, 2356); F3, 1825 (1226, 2848); F4, 12,161 (5614, 22,112). The ˜14-fold increase in total serum BA in NASH cirrhosis consisted primarily of an increase in conjugated BAs 11,518 (4737, 17,572) compared with F2 NASH, 1141 (431, 1714). Primary conjugated bile acids [glycocholate (GCA), taurocholate (TCA), glycodeoxycholate (GCDCA), and taurodeoxycholate (TCDCA)] were the major primary BA species elevated in F4 vs F2 NASH. GCA and TCA were ˜15 and 35-fold higher, respectively, in F4 compared to F2 NASH.

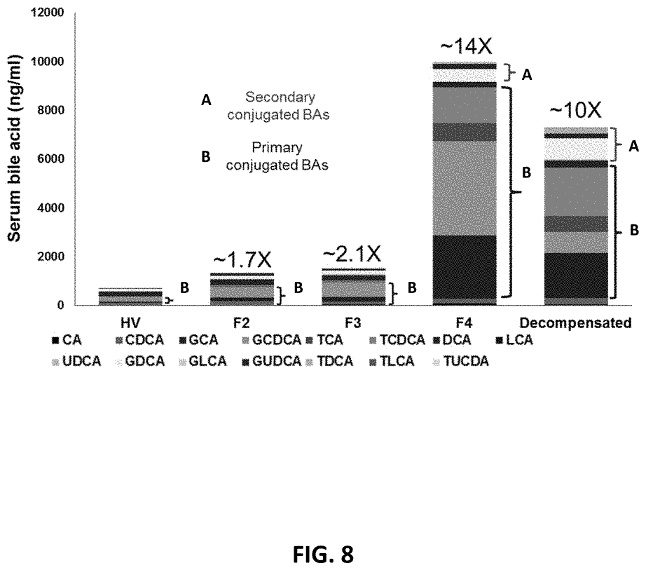

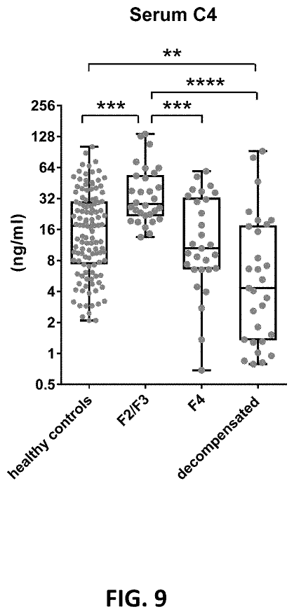

Clinical decompensation of NASH cirrhosis subjects led to decreased BA levels, but at levels still ˜10-fold higher than controls (FIG. 8). In contrast, BA synthesis as reflected in serum C4 levels declined with the development of cirrhosis and further decreased with clinical decompensation (FIG. 9). C4 levels in F2/F3 patients, 28.4 ng/mL (21.5, 54.1), were significantly higher than controls, 17.4 (7.3, 30.0). Development of cirrhosis resulted in C4 levels that were 63% lower than F2/F3 patients, 10.5 (6.6, 32.8) and decreased further with clinical decompensation, 4.3 (1.3, 17.6).

An increase in overall BAs and conjugated primary BAs occurs in F2/F3 NASH compared to healthy controls and is accompanied by elevated C4 levels. In NASH cirrhosis, pronounced elevation in BAs occurs despite lower C4 levels than those found in F2/F3 NASH. These results indicate that the mechanisms responsible for BA homeostasis are lost in cirrhosis, and become further dysregulated with clinical decompensation.

### Example 3. Extracellular Proteome-RNAseq Analysis for Identifying Non-Invasive Nash-Fibrosis Biomarker

This example used a combination of NASH biopsy-derived transcriptomics analysis and predictive bioinformatics algorithms to identify 100 transcripts that could produce secreted/leaked proteins (so called NASH secretome). These transcripts exhibited fibrosis stage dependent expression profiles and are relevant to NASH biology. Individual or combined transcripts are good discriminators (AUROC >0.86) for classifying NASH subjects with cirrhosis (F4) or severe fibrosis (F3/F4).

ELISA assays were selected and qualified for the top 30 candidates. 11 proteins (YKL-40, FAP, ITGB6, EMILIN1, FNDC1, IGDCC4, MASP2, SCF, LTBP2, ADAMTS12 and MCM2) exhibited significant association with CRN fibrosis in F0-F4 samples. FNDC1 and MCM2 (AUROC=0.80 and 0.76, respectively) are good discriminators of severe fibrosis (F≥3). Longitudinal changes of the 11 markers are not associated with fibrosis improvement or worsening. Changes in YKL-40, FAP, and SCF (AUROC=0.70, 0.67 and 0.72, respectively) are fair monitoring markers of steatosis improvement. Baseline levels of FAP, SCF, and LTBP2 are fair prognostic markers (AUROC=0.78, 0.71 and 0.70, respectively) of fibrosis improvement in F4 subjects.

In Example 1, assays for selective protein targets (LOXL2, Lumican, TGFBI, CK-18s, Pro-C3) as well as high-content proteomic platform (SOMAscan) were employed as part of the comprehensive proteomic approaches to identify and evaluate novel protein markers for NASH. Even though about 1350 proteins were covered by these two approaches, there are still many potential circulating proteins that are not included. As a complementary proteomic approach, NASH biopsy-derived transcriptomics analysis was combined with predictive bioinformatics algorithms to identify additional secreted/leaked proteins (so called NASH secretome) from liver for further exploration.

Proteins in circulation may come from 1) “Classic secretory” via exocytosis, 2) “Non-classic secretory” through translocation, lysosomal secretion or exosome, or 3) tissue leakage due to cell death or damage.

In order to predict changes in potential circulating proteins, transcriptomic information from tissues of interest to predict potential secreted or leaked protein. This example developed bioinformatics algorithms to predict genes to 1) demonstrate fibrosis stage dependent differential expression in NASH subjects and 2) encode for secreted proteins. These NASH secretome will represent potential tissue-selective and disease severity dependent candidates as circulating protein biomarkers. After these candidates were identified, protein quantification data were either generated using ELISA or derived from SOMAscan if available.

### Samples

Procured FFPE liver samples listed in Table 5 were used for RNA extraction and RNAseq

TABLE 5
Procured Samples for RNAseq

| | Healthy | F1 | F2 | F3 | F4 |
|---|---|---|---|---|---|
| Phase I: pilot (completed) | 6* | 4 | 14 | 11 | 1 |
| Phase 2: second dataset | | | | | |
| (initiated, data expected in | 21 | 46 | 27 | 5 | 10 |
| May) | | | | | |
| Total | 27 | 50 | 41 | 16 | 11 |

### ELISA Testing for Selected Candidates

Procures serum sample were used for initial ELISA screening experiments to identify candidates for further testing using clinical study samples.

### Bioinformatics Methods for Candidate Selection

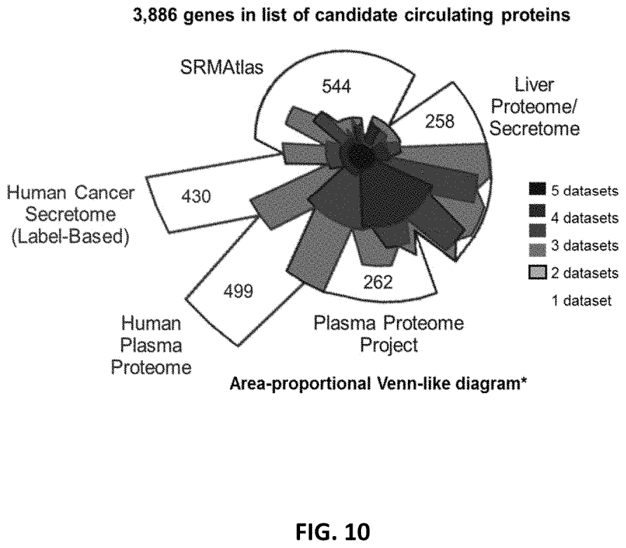

Genes with experimental/MS evidence of secretion were identified in public databases/datasets. Collectively, about 3886 potential secreted or leaked proteins were identified and overlapped or unique proteins from each dataset are illustrated in FIG. 10.

A candidate list of non-invasive, circulating biomarkers for NASH progression using RNA-seq data and public databases/datasets was then compiled with a bioinformatic workflow.

### NASH Secretome Experimental Workflow

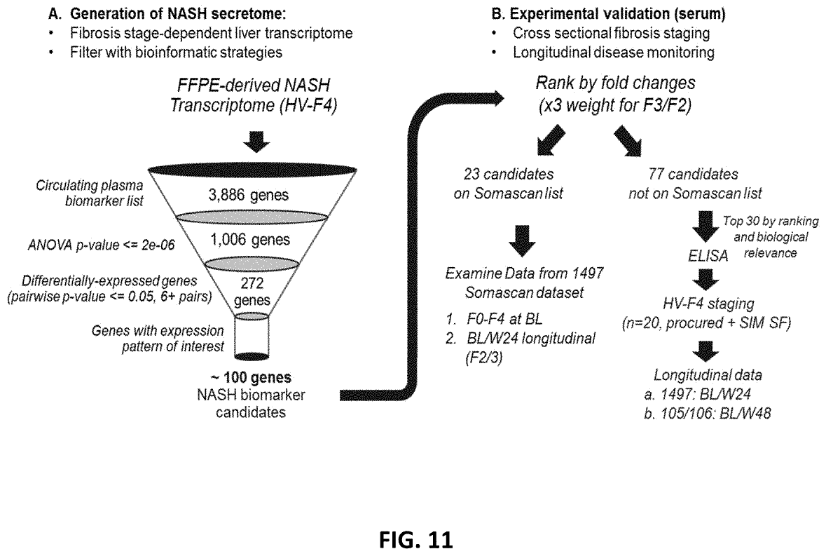

About 100 genes were selected after the filtering procedures mentioned above. These potential fibrosis stage-dependent, liver selective secreted proteins were then going through experimental validation either by SOMAscan (depends on availability) or ELISA testing (FIG. 11).

### Objectives

As a complementary proteomic approach, bioinformatics algorithms were employed to predict potential hepatic secreted/leaked proteins from NASH subjects. Circulating levels of promising candidates are measured using ELISA assays or SOMAscan (depends on availability) to examine association of: levels of these protein markers with fibrosis stages at BL; longitudinal changes of these markers with improvement/worsening of liver fibrosis in NASH subjects.

Evaluate the performance (i.e., AUROC) of markers from secretome panel for diagnosing advanced fibrosis (F3-4 vs. F0-2) and cirrhosis (F4 vs. F0-3) in combined datasets.

Evaluate the performance of markers secretome panel for monitoring CRN fibrosis stage change.

Evaluate the performance of markers secretome panel for prognosing CRN fibrosis stage change (improvement and worsening).

Evaluate the performance of markers secretome panel for monitoring improvement in NAS components (hepatic ballooning, lobular inflammation and steatosis).

Evaluate the performance of markers secretome panel for diagnosing NASH vs. non-NASH in combined dataset across studies SEL1497, SIM 0105 and 0106, where non-NASH is evaluated using the following four definitions: 1) 0 in any NAS subscore (lobular inflammation, hepatic ballooning and steatosis), 2) no to mild inflammation (NASLI≤1), 3) no ballooning (NASHB=0), 4) no active NASH (NASLI≤1 and NASHB=0).

### Statistical Methods

For AUROC calculation for binary endpoint, the method of repeated cross-validation was performed. Specifically, the data is randomly divided into 5-folds, using 4 of the folds for the modeling (i.e. logistic regression) and predicting on the left-out fold, and performing the modeling/predicting process for each of the 5-folds until the predictions are obtained for all 5 folds (i.e. whole dataset). AUROC performance metric is obtained. The cross-validation procedure is then repeated 100 times with a different randomly-divided 5 folds each time, and the mean and 95% CI for the AUROC are provided across the 100 repeats. In the modeling, logistic regression was used to model the binary endpoint (e.g. baseline F4 vs. F0-3) with the biomarker(s) as covariate.

Additional analyses for baseline CRN fibrosis stage are provided. In particular, boxplots of baseline levels of specific conventional tests by baseline CRN fibrosis stage are provided. The Jonckheere-Terpstra trend test was conducted to assess the trend (whether increasing or decreasing) of biomarker levels with fibrosis stage.

### Data

100 NASH Secretome candidate genes were generated after the bioinformatics filters were applied. Among these 100 targets, many code for proteins having functions related to NASH biology (Table 6).

TABLE 6
Secretome Candidates Participate in NASH Relevant Biological Pathways

| | | Cytoskeleton | | | Platelet/ | | |
|---|---|---|---|---|---|---|---|
| | Membrane | myosin | Neuro- | Immunoglobulin | complement | Enzyme/ | |
| ECM | receptors | related | endocrine | cyto/chemokine | related | TF | Others |
| CoIA1 | EPHA3 | MYH10 | TENM4 | IGDCC4 | THBS2 | PLCE1 | FAM129B |
| CoI3A1 | EPHB2 | MY05A | SYTL2 | IGSF3 | C4BPB | FUBP1 | FNDC1 |
| CoI4A1 | FGFR2 | TPM1 | TANC1 | PRTG | C4BPA | AEBP1 | HPX |
| CoI4A2 | FGFR3 | LIMA1 | NEO1 | SCF | MASP2 | ZNF671 | GC |
| CoI5A1 | DDR1 | CALD1 | ROBO1 | CCL14 | TFPI | PTGDS | AGT |
| CoI6A3 | TLR7 | PODN | UNC5B | | | ZNF101 | CPN2 |
| CoI14A1 | LTBP2 | SPTAN1 | THY1 | | | LUZP1 | CLEC3B |
| CHI3L1 | MRC2 | KIF23 | NAV3 | | | ATP5J2 | APOC3 |
| LOXL1 | ITGB6 | DPYSL3 | PCLO | | | FAP | APOH |
| LAMC2 | MARCO | TNS3 | LRRC4B | | | CPB2 | APCS |
| EMILIN1 | LYVE1 | UTRN | CADPS | | | ALDH6A1 | |
| CDH6 | | | AHNAK | | | PCBD1 | |
| PCDH18 | | | | | | PRDX6 | |
| ADAMTSL2 | | | | | | PON3 | |
| ADAMS12 | | | | | | SMPDL3A | |
| AGRN | | | | | | GLO1 | |
| DNAH7 | | | | | | PSMB2 | |
| CCDC80 | | | | | | CTH | |
| YKL-40 | | | | | | MOCS2 | |
| FCN3 | | | | | | SERPINF2 | |
| FCN2 | | | | | | KIAA1161 | |

* bold and underlined: levels decreased with increased fibrosis stage; otherwise levels decreased with increased fibrosis stage.

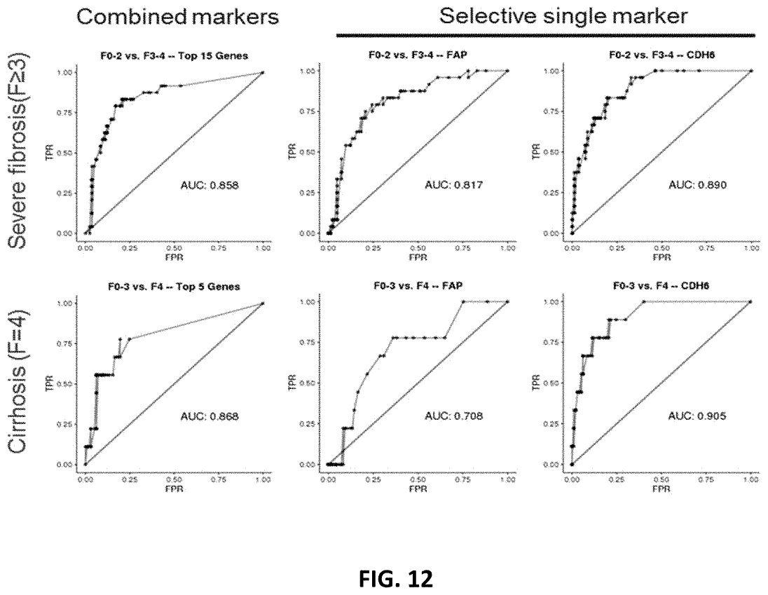

This example then performed analyses to ask whether hepatic expressions of these genes can be used as classifiers for severe fibrosis and/or cirrhosis. It was noticed that both single (FAP or CDH6) and combined genes (panel of 15 for F3, panel of 5 for F4) are good classifier for severe fibrosis or cirrhosis (FIG. 12).

However, utilizing hepatic RNA as NASH biomarkers is not practical, circulating protein levels of these candidates are further explored to be candidates for diagnostic and/or disease monitoring markers for fibrosis in NASH.

Two approaches were taken to generate circulating protein data for selected Secretome candidates, SOMAscan and ELISA.

### 1. SOMAscan

There are 23 Secretome candidates included in the SOMAscan platform.

TABLE 7
Secretome Candidates Included in SOMAascan

| Protein | Gene | Full name |
|---|---|---|
| TSP2 | THBS2 | Thrombospondin-2 |
| MRC2 | MRC2 | C-type mannose receptor 2 |
| SAP | APCS | Serum amyloid P-component |
| EPHB2 | EPHB2 | Ephrin type-B receptor 2 |
| SPTA2 | SPTAN1 | Spectrin alpha chain, non-erythrocytic 1 |
| A2AP | SERPINF2 | Alpha 2-antiplasmin |
| CDH6 | CDH6 | Cadherin-6 |
| CCDC80 (URB) | CCDC80 | Coiled-coil domain-containing protein 80 |
| CCL14 (HCC-1) | CCL14 | C-C motif chemokine 14 |
| CPB2 (TAFI) | CPB2 | Carboxypeptidase B2 |
| DDR1 | DDR1 | Epithelial discoidin domain- |
| | | containing receptor 1 |
| EPHA3 | EPHA3 | Ephrin type-A receptor 3 |
| FCN2 | FCN2 | Ficolin-2 |
| FCN3 | FCN3 | Ficolin-3 |
| FGFR2 | FGFR2 | Fibroblast growth factor receptor 2 |
| FGFR3 | FGFR3 | Fibroblast growth factor receptor 3 |
| HPX | HPX | Hemopexin |
| KIF23 | KIF23 | kinesin family member 23provided |
| LYVE1 | LYVE1 | Lymphatic vessel endothelial hyaluronic |
| | | acid receptor 1 |
| PRDX6 | PRDX6 | Peroxiredoxin 6 |
| ASM3A | SMPDL3A | Acid sphingomyelinase-like |
| | | phosphodiesterase 3a |
| TPM1 | TPM1 | Tropomyosin 1 alpha chain |
| TFPI | TFPI | Tissue factor pathway inhibitor |

Circulating levels of the coded proteins for these 23 genes were examined from the SOMA logic dataset in Example 1 and it was found that:
TSP2, MRC2, SAP, EPHB2, SPTA2, and SEPR were significantly (p<0.05) associated with CRN fibrosis stage.
No significant associations identified between changes in these markers with improvements in fibrosis stage, however, change in A2AP showed potential disease monitoring characteristics.
Changes in TSP2, MRC2, SAP, EPHB2, HPX, and LYVE1 were associated with steatosis improvement (Table 8).

TABLE 8
Changes in Secretome Candidate Markers with
Improvements in Fibrosis or NAS Components in
SOMAscan Dataset (1497; BL/Week 24)

| | Fibrosis | Steatosis | LI | HB |
|---|---|---|---|---|
| THBS2(TSP2) | P = 0.47 | P < 0.001 | P < 0.05 | P = 0.085 |
| MRC2 | P = 0.84 | P < 0.05 | P = 0.67 | P = 0.41 |
| APCS(SAP) | P = 0.44 | P < 0.001 | P = 0.25 | P = 0.63 |
| EPHB2 | P = 0.62 | P < 0.05 | P = 0.66 | P = 0.67 |
| CPB2(TAFI) | P = 0.52 | P = 0.59 | P < 0.05 | P = 0.55 |
| DDR1(discoidin | P = 0.51 | P = 0.63 | P < 0.05 | P = 0.87 |
| domain receptor | | | | |
| 1) | | | | |
| HPX(hemopexin) | P = 0.72 | P < 0.005 | P = 0.28 | P = 0.24 |
| KIF23 | P = 0.14 | P = 0.30 | P < 0.01 | P = 0.93 |
| LYVE1 | P = 0.41 | P < 0.005 | P < 0.01 | P = 0.38 |

### 2. ELISA Assays

ELISA assays were developed and qualified for secretome candidates that were not included on the SOMAscan (ITGB6, FNDC1, MCM2, EMILIN1, IGDCC4, MASP2, SCF, LTBP2, ADAMTS12) as well as for those (TSP2, A2AP, MRC2, SAP, CTSH, IGFBP7, C7, MAC2BP) that were on SOMAscan platform and demonstrated promising preliminary results on fibrosis staging associations (Table 9).

TABLE 9
ELISA Assays Developed for Secretome Candidates

| Protein | Gene | Full name | Biological function |
|---|---|---|---|
| CHI3L1 (YKL- | CHI3L1 | Chitinase-3-like protein 1 | ECM glycoprotein |
| 40) | | | |
| FAP | FAP | Fibroblast activation protein | Membrane-bound |
| | | | protease |
| ITGB6 | ITGB6 | Integrin, beta 6 | Membrane receptor |
| FNDC1 | FNDC1 | Fibronectin type III domain | G-protein signaling |
| | | containing 1 | |
| MCM2 | MCM2 | Mini-chromosome | Genome |
| | | maintenance protein 2 | replication/liver |
| | | | regeneration |
| EMILIN1 | EMILIN1 | Elastin microfibril interfacer 1 | ECM glycoprotein |
| IGDCC4 | IGDCC4 | Immunoglobulin superfamily | Immunoglobulin |
| | | DCC subclass member 4 | |
| MASP2 | MASP2 | Mannan-binding lectin serine | Protease to cleave |
| | | protease 2 | complement(C4/C2) |
| SCF(Kit ligand) | KITLG | Kit ligand | Cytokine |
| LTBP2 | LTBP2 | Latent-transforming growth | ECM; binds to TGF [symbol] |
| | | factor beta-binding protein 2 | Extracellular |
| ADAMTS12 | ADAMTS12 | A disintegrin and | protease; |
| | | metalloproteinase with | promote |
| | | thrombospondin motifs 12 | fibrogenesis |
| TSP2 | THBS2 | Thrombospondin-2 | Glycoprotein; mediates |
| | | | cell-to-cell and cell-to- |
| | | | matrix interactions |
| A2AP | SERPINF2 | Alpha 2-antiplasmin | Serine protease inhibitor |
| MRC2 | MRC2 | C-type mannose receptor 2 | Endocytotic receptor; |
| | | | internalizes glycosylated |
| | | | ligands |
| SAP | APCS | Serum amyloid P-component | Acute phase proteins (APP) |
| CTSH | CTSH | Cathepsin H | lysosomal cysteine |
| | | | proteinase |
| IGFBP7 | IGFBP7 | Insulin-like growth factor- | Controls |
| | | binding protein 7 | availability of IGFs |
| | | | to tissue/cells |
| C7 | C7 | Complement C7 | Serum glycoprotein, |
| | | | complement complex |
| | | | component |
| MAC2BP | LGALS3BP | Galectin-3-binding protein | Modulates cell-cell |
| | | | and cell-matrix |
| | | | interactions |

### Results

Association of Circulating Secretome Candidates with Fibrosis Stages

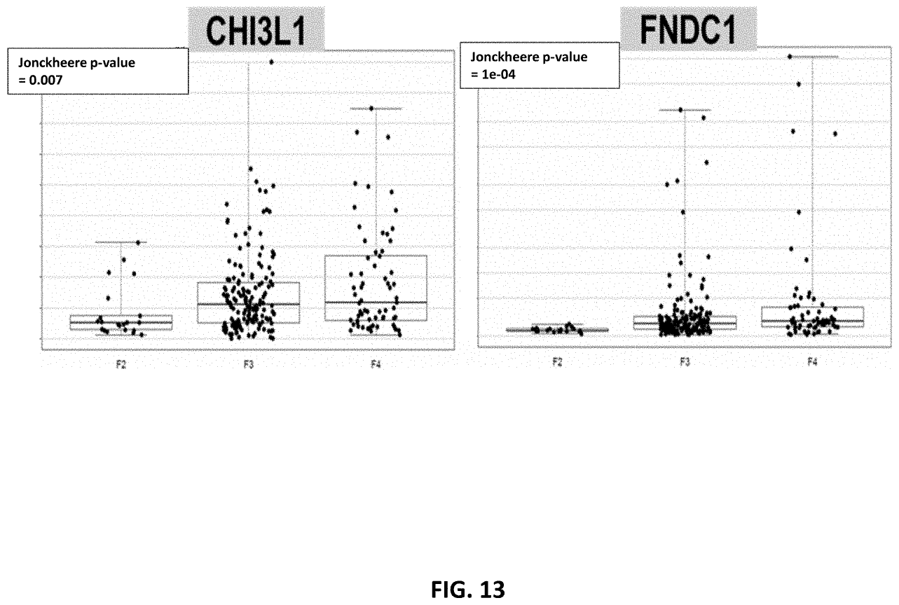

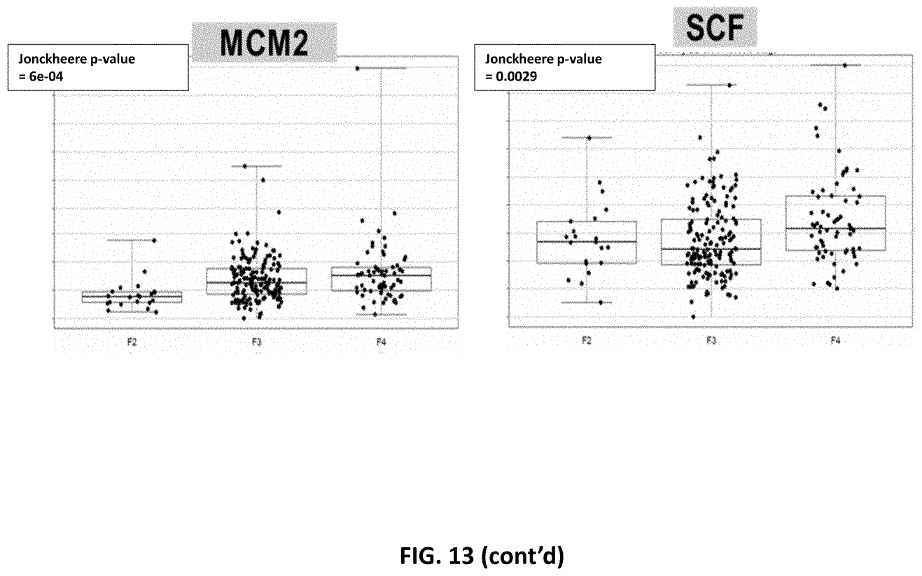

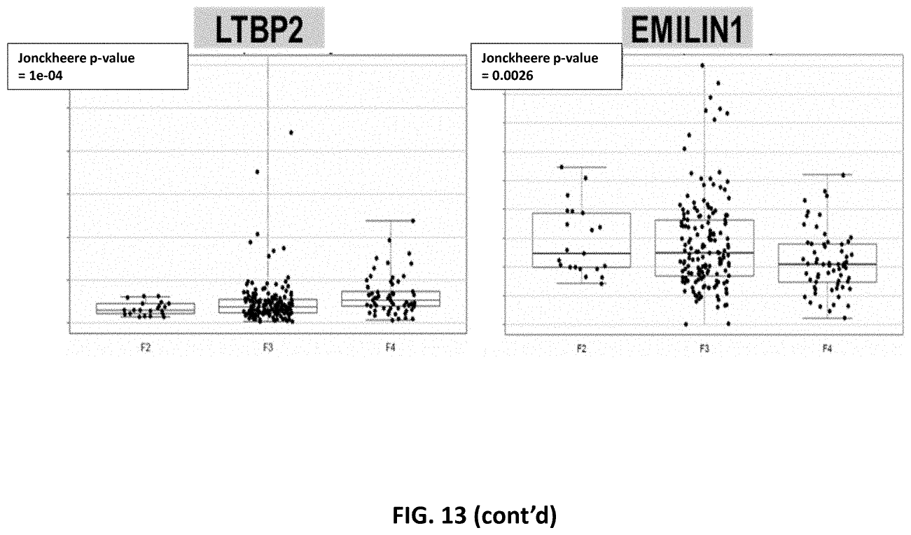

ELISA assays were performed in batches, first 11 candidates (CHI3L1, FAP, ITGB6, FNDC1, MCM2, EMILIN1, IGDCC4, MASP2, SCF, LTBP2, ADAMTS12) were tested and 6 out of 11 candidates demonstrated significant association with fibrosis stage (FIG. 13).

Due to limitation in 1497 study samples, 8 additional secretome candidates (TSP2, A2AP, MRC2, SAP, CTSH, IGFBP7, C7, MAC2BP) were examined using additional samples both at baseline and at week 48. 5 out of 8 candidates demonstrated significant association with fibrosis stage, including TSP2, A2AP, SAP, IGFBP7 and C7.

These results showed that the bioinformatics predictive algorism developed here successfully identified potential hepatic secreted/leaked proteins that correlate with NASH disease severity and could have potential for diagnosing and monitoring fibrosis in NASH.

### Diagnosis of CRN Fibrosis Stage (Advanced Fibrosis [F3, 4] and Cirrhosis [F4])

In order to evaluate the performance of these novel protein candidates on staging fibrosis in NASH subjects, univariate and multivariate analysis were used to identify individual markers or marker sets that can classify subjects with advanced fibrosis (F>3) or with cirrhosis and the results are summarized below.

AUROC≥0.7 was observed for diagnosing advanced fibrosis for IGFBP7 and TSP2, and for diagnosing cirrhosis for SAP, A2AP and IGFBP7 using baseline and wk48 data [Table 16.8.2.1]. C7 had AUROC of 0.65 for diagnosing cirrhosis.

Differences were observed in AUROC for diagnosing baseline advanced fibrosis and cirrhosis using baseline and screen fail data and baseline, screen fail and wk48 data due to small sample size in F0-2 in baseline and screen fail data (Table 10).

TABLE 10
Evaluation of Secretome Panel for Diagnosing Baseline Advanced Fibrosis
(F3-4 vs. F0-2) and Cirrhosis (F4 vs. F0-3) in Enrolled, Screen-Failed and
Week 48 Data from Combined Studies

| | AUC (95% CI)* | | | | | | |
|---|---|---|---|---|---|---|---|
| | | | | | | BL/SF + | BL/SF + |
| | BL + SF | BL + SF | | Wk48 | Wk48 | Wk48 | Wk48 |
| | (3 studies) | (3 studies) | Wk24 | (105/106) | (105/106) | (3 studies)** | (3 studies)** |
| | F3, 4 vs. F0, | F4 vs. F0, | (1497) | F3, 4 vs. F0, | F4 vs. F0, | F3, 4 vs. F0, | F4 vs. F0, |
| Test | 1, 2 | 1, 2, 3 | F3 vs. F1, 2 | 1, 2 | 1, 2, 3 | 1, 2 | 1, 2, 3 |
| ADAMTS12 | 0.54 | 0.53 | 0.52 | 0.58 | 0.53 | 0.51 | 0.54 |
| | (0.49, 0.57) | (0.49, 0.55) | (0.47, 0.62) | (0.52, 0.65) | (0.48, 0.55) | (0.48, 0.55) | (0.52, 0.55) |
| CHI3L1 | 0.66 | 0.55 | 0.52 | 0.59 | 0.63 | 0.63 | 0.6 |
| | (0.64, 0.68) | (0.49, 0.57) | (0.47, 0.59) | (0.56, 0.62) | (0.61, 0.64) | (0.6, 0.64) | (0.59, 0.61) |
| EMILIN1 | 0.53 | 0.62 | 0.58 | 0.59 | 0.56 | 0.57 | 0.6 |
| | (0.45, 0.58) | (0.61, 0.62) | (0.44, 0.67) | (0.57, 0.61) | (0.54, 0.57) | (0.54, 0.59) | (0.59, 0.6) |
| FAP | 0.55 | 0.51 | 0.66 | 0.64 | 0.71 | 0.54 | 0.58 |
| | (0.48, 0.6) | (0.48, 0.57) | (0.63, 0.68) | (0.62, 0.65) | (0.69, 0.71) | (0.51, 0.56) | (0.57, 0.58) |
| FNDC1 | 0.81 | 0.57 | 0.58 | 0.58 | 0.54 | 0.56 | 0.55 |
| | (0.8, 0.81) | (0.51, 0.59) | (0.56, 0.61) | (0.48, 0.66) | (0.47, 0.61) | (0.53, 0.59) | (0.52, 0.57) |
| IGDCC4 | 0.57 | 0.52 | 0.6 | 0.53 | 0.52 | 0.51 | 0.52 |
| | (0.54, 0.62) | (0.48, 0.58) | (0.51, 0.69) | (0.47, 0.58) | (0.47, 0.56) | (0.48, 0.56) | (0.48, 0.55) |
| ITGB6 | 0.56 | 0.54 | 0.55 | 0.51 | 0.57 | 0.52 | 0.56 |
| | (0.48, 0.68) | (0.5, 0.57) | (0.46, 0.67) | (0.44, 0.55) | (0.54, 0.59) | (0.48, 0.59) | (0.54, 0.57) |
| MASP2 | 0.59 | 0.53 | 0.59 | 0.53 | 0.52 | 0.52 | 0.52 |
| | (0.56, 0.65) | (0.48, 0.58) | (0.47, 0.69) | (0.48, 0.58) | (0.48, 0.56) | (0.49, 0.57) | (0.49, 0.55) |
| SCF | 0.55 | 0.63 | 0.55 | 0.59 | 0.6 | 0.57 | 0.63 |
| | (0.47, 0.64) | (0.62, 0.64) | (0.46, 0.66) | (0.5, 0.66) | (0.57, 0.61) | (0.52, 0.63) | (0.62, 0.63) |
| LTBP2 | 0.62 | 0.65 | 0.59 | 0.53 | 0.54 | 0.58 | 0.53 |
| | (0.6, 0.63) | (0.63, 0.67) | (0.53, 0.63) | (0.47, 0.59) | (0.48, 0.61) | (0.53, 0.61) | (0.48, 0.58) |
| MCM2 | 0.76 | 0.58 | 0.52 | 0.51 | 0.56 | 0.57 | 0.59 |
| | (0.74, 0.77) | (0.56, 0.59) | (0.45, 0.64) | (0.48, 0.56) | (0.54, 0.58) | (0.55, 0.58) | (0.58, 0.59) |
| FNDC1 + MCM2 | 0.84 | 0.6 | 0.54 | 0.55 | 0.53 | 0.57 | 0.59 |
| | (0.83, 0.86) | (0.57, 0.61) | (0.43, 0.61) | (0.51, 0.6) | (0.47, 0.56) | (0.55, 0.59) | (0.58, 0.6) |
| FNDC1 + MCM2 + | 0.86 | 0.77 | 0.65 | 0.78 | 0.8 | 0.72 | 0.8 |
| BA + NFS | (0.83, 0.87) | (0.75, 0.78) | (0.59, 0.7) | (0.75, 0.8) | (0.77, 0.82) | (0.7, 0.74) | (0.79, 0.81) |
| | AUC (95% CI)* | | | | | | |
| | | | | | | BL + | BL + |
| | | | Wk24 | | | Wk48 | Wk48 |
| | BL | BL | (1497) | | | F3, 4 vs. F0, | F4 vs. F0, |
| Test | F3, 4 vs. F2 | F4 vs. F3 | F3 vs. F1, 2 | | | 1, 2 | 1, 2, 3 |
| TSP2 | | 0.63 | | 0.7 | 0.71 | 0.71 | 0.67 |
| | | (0.61, 0.64) | | (0.69, 0.72) | (0.7, 0.72) | (0.69, 0.72) | (0.66, 0.67) |
| A2AP | | 0.71 | | 0.67 | 0.72 | 0.66 | 0.71 |
| | | (0.7, 0.72) | | (0.65, 0.68) | (0.71, 0.72) | (0.64, 0.66) | (0.71, 0.72) |
| MRC2 | | 0.58 | | 0.59 | 0.52 | 0.58 | 0.52 |
| | | (0.47, 0.65) | | (0.47, 0.65) | (0.47, 0.58) | (0.47, 0.65) | (0.47, 0.58) |
| SAP | | 0.78 | | 0.62 | 0.77 | 0.59 | 0.77 |
| | | (0.77, 0.78) | | (0.59, 0.63) | (0.76, 0.77) | (0.56, 0.61) | (0.77, 0.78) |
| CTSH | | 0.5 | | 0.55 | 0.52 | 0.52 | 0.51 |
| | | (0.48, 0.53) | | (0.52, 0.58) | (0.43, 0.57) | (0.47, 0.55) | (0.46, 0.55) |
| IGFBP7 | | 0.63 | | 0.76 | 0.78 | 0.75 | 0.71 |
| | | (0.6, 0.64) | | (0.75, 0.77) | (0.77, 0.78) | (0.74, 0.76) | (0.7, 0.71) |
| C7 | | 0.72 | | 0.54 | 0.56 | 0.53 | 0.65 |
| | | (0.71, 0.73) | | (0.5, 0.56) | (0.53, 0.57) | (0.49, 0.58) | (0.64,0.65) |
| MAC2BP | | 0.54 | | 0.6 | 0.61 | 0.59 | 0.55 |
| | | (0.49, 0.59) | | (0.58, 0.62) | (0.59, 0.62) | (0.56, 0.61) | (0.53, 0.56) |

*Denotes mean and corresponding 95% CI for 5-fold cross-validation repeated 100 times.
**BL/SF from all 3 studies, but wk48 values from only SIM 105/106 (analyzing all data together).

### Monitoring of CRN Fibrosis Stage Change (Improvement or Worsening)

This example next examined the longitudinal changes of these novel protein markers and asked whether these markers can have potential values for monitoring fibrosis stage changes and the results are summarized below.

Using only % change from baseline as predictors, several markers have AUROC between 0.60-0.70 for monitoring fibrosis stage changes but none of the markers from secretome panel have performance of AUROC≥0.7 for monitoring CRN fibrosis improvement or CRN fibrosis worsening.

In general, the performance of AUROC is improved when using both baseline and % change from baseline compared to using only change from baseline as predictors. FAP, LTBP2, SAP, IGFBP7, and MAC2BP have AUROC>0.70 for monitoring CRN fibrosis improvement or CRN fibrosis worsening (SIM 105).

### Prognosing of CRN Fibrosis Stage Change (Improvement or Worsening at Week 48) Using Baseline Biomarker Levels

This example also performed analysis to address whether baseline levels of these markers has prognosis values to predict week 48 fibrosis stage changes in SIM study samples since it is believed these studies are considered as nature progression due to lack of efficacy by SIM. The results are summarized below.

Several Markers (Using Baseline Levels) have Performance of AUROC≥0.7 for Predicting.

CNR fibrosis improvement: IGFBP7 (F3 to lower at wk48 in SIM105, F4 to lower at wk48 in SIM106, F4/3 to lower at wk48), FAP and SCF (F4 to lower at wk48).

CRN fibrosis worsening (F3 to F4 at wk48): IGFBP7 and SAP.

### Monitoring of Changes (Improvement or Worsening) in NAS Score Components

Using % change from baseline as predictors, several markers have AUROC between 0.60-0.70 for monitoring improvement in NAS component (hepatic ballooning, lobular inflammation or steatosis).

In general, the performance of AUROC for monitoring improvement in NAS component is improved when using both baseline and change from baseline as predictors. AUROC≥0.7 was observed for FAP for monitoring lobular inflammation improvement (Grade 3 to lower at wk48), and ADAMTS12 for monitoring steatosis improvement (Grade 2/3 to lower at wk48).

Diagnosis of NASH vs. Non-NASH in SEL 1497, SIM 0105 and 0106 Studies

Using data from baseline and wk48, TSP2 had AUROC of 0.66/0.68/0.7/0.71 for diagnosing NASH vs. non-NASH [4 definitions of non-NASH: 1) 0 for any NAS subscore (lobular inflammation, hepatic ballooning or steatosis); 2) no to mild inflammation; 3) no ballooning; 4) no active NASH (no to mild inflammation and no ballooning)]. Similar AUROC was observed when using only baseline data from enrolled patients in combined SIM105/106 studies.

### Conclusions

In this example, a new approach was taken to generate potential liver selective and fibrosis stage dependent protein biomarkers using biopsy derived transcriptome data and bioinformatic algorisms to predict secreted/leaking proteins. This strategy was proven to be fruitful with the following key findings, (1) candidate genes code for proteins that are involved in fibrosis biology; (2) hepatic expression profiles of these genes (single or selective panel) have good classifying characteristics (AUROC 0.8-0.9) for identification of severe fibrosis (F3) or cirrhosis (F4); and (3) circulating levels of selective candidates were evaluated either by SOMAscan (depends on availability) or ELISA assays.

AUROC≥0.7 was observed for diagnosing advanced fibrosis for IGFBP7 and TSP2, and for diagnosing cirrhosis for SAP, A2AP and IGFBP7 using baseline, screen fail and wk48 data. C7 had AUROC of 0.65 for diagnosing cirrhosis.

None of the markers from secretome panel (using % change from baseline) had AUROC≥0.7 for monitoring CRN fibrosis changes (either improvement or worsening) and improvement in NAS component (hepatic ballooning, lobular inflammation, and steatosis). In general, the performance of AUROC is improved when using both baseline and % change from baseline.

Using baseline levels, several markers had AUROC≥0.7 for predicting:

CRN fibrosis improvement: IGFBP7 (F3 to lower at wk48, F4 to lower at wk48, F4/3 to lower at wk48), FAP and SCF (F4 to lower at wk48); and

Fibrosis worsening (F3 to F4 at wk48): IGFBP7 and SAP.

Using data from baseline and wk48 in combined SIM105/106 studies, TSP2 had AUROC of ˜0.7 for diagnosing NASH vs. non-NASH across the 4 definitions.

### Example 4. Additional SOMAscan Data for GDF-15 and CD163

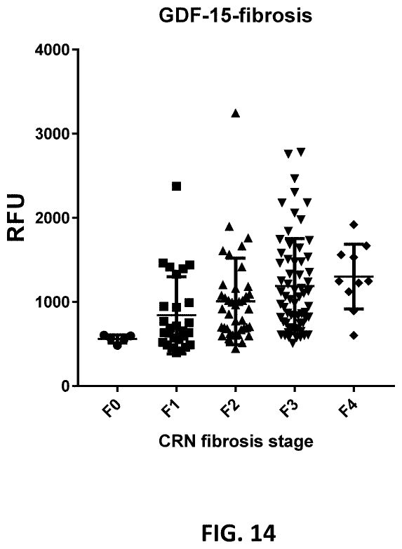

This example reports additional SOMAscan data collected with the method in Example 1, for the circulating proteins GDF-15 and CD163. Summary data are presented in charts in FIG. 14-18.

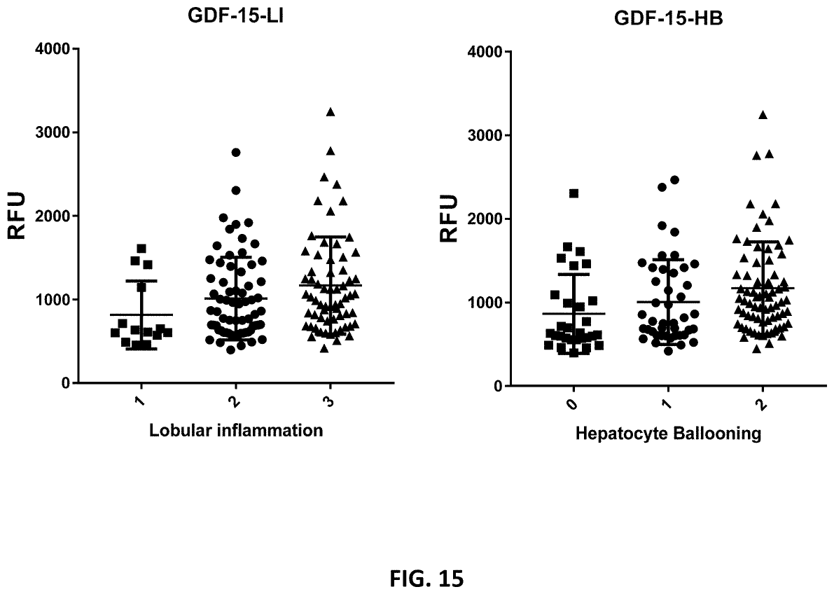

As shown in FIG. 14, circulating GDF-15 levels were significantly associated with fibrosis stage in NASH subjects; p<0.0001 (Kruskal-Wallis test). Likewise, as shown in FIG. 15, the circulating GDF-15 levels were significantly associated with Lobular inflammation (left panel; P<0.05 (Kruskal-Wallis test)) and Hepatic Ballooning in the NASH subjects (right panel; P<0.005 (Kruskal-Wallis test)). However, the circulating GDF-15 levels were not associated with steatosis or NAS scores in the NASH subjects.

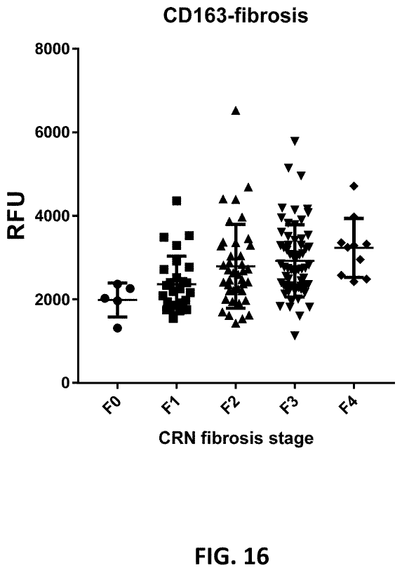

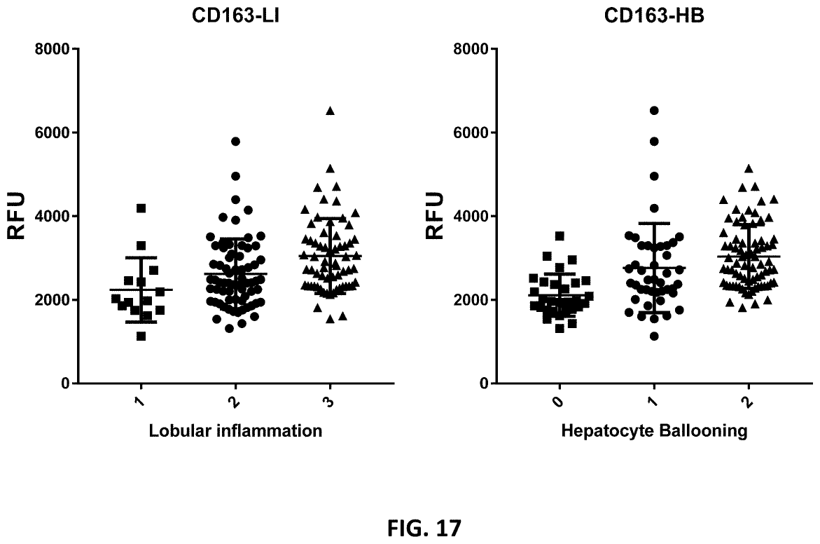

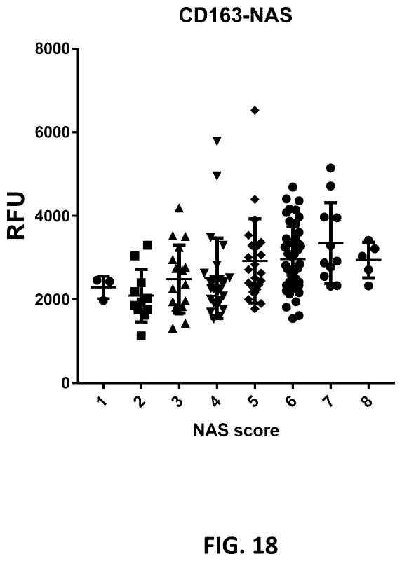

The chart is FIG. 16 shows that circulating CD163 levels were significantly associated with fibrosis stages in NASH subjects, p<0.001 (Kruskal-Wallis test). The circulating CD163 levels were also significantly associated with Lobular inflammation (FIG. 17, left panel, p<0.0005 (Kruskal-Wallis test)) and Hepatic Ballooning (FIG. 17, right panel, p<0.0001 (Kruskal-Wallis test)) in the NASH subjects. The circulating CD163 levels were also significantly associated with NAS scores (FIG. 18, p<0.001 (Kruskal-Wallis test)) but not with steatosis in the NASH subjects.

The results of Examples 3 and 4 are summarized in the following tables, complementary to Tables 1-4.

### Tables 11A-D: Protein Markers for Diagnosis

TABLE 11A
Protein Markers for CRN Fibrosis Stages (CRN)

| | | | CRN | CRN | CRN | CRN | CRN |
|---|---|---|---|---|---|---|---|
| Marker | | | fibrosis | fibrosis | fibrosis | fibrosis | fibrosis |
| Name | UniProt | p value | stage 0 | stage 1 | stage 2 | stage 3 | stage 4 |
| YKL-40 | P36222 | <0.01 | N/A | N/A | 63100 | 124716 | 129864 |
| FAP | Q12884 | 0.606 | N/A | N/A | 50761 | 44068 | 44705 |
| ITGB6 | P18564 | 0.202 | N/A | N/A | 14.5 | 14.0 | 17.1 |
| EMILIN1 | Q9Y6C2 | <0.005 | N/A | N/A | 16802 | 16818 | 14941 |
| FNDC1 | Q4ZHG4 | <0.0005 | N/A | N/A | 0.4 | 1.0 | 1.2 |
| IGDCC4 | Q8TDY8 | 0.076 | N/A | N/A | 321.2 | 274.1 | 262 |
| MASP2 | O00187 | 0.127 | N/A | N/A | 718.8 | 579.5 | 591.8 |
| SCF | P21583 | <0.005 | N/A | N/A | 732.3 | 693.7 | 798.8 |
| LTBP2 | Q14767 | <0.0005 | N/A | N/A | 1.5 | 1.9 | 2.6 |
| ADAMTS12 | P58397 | 0.199 | N/A | N/A | 15825.8 | 19191 | 25317.7 |
| MCM2 | P49736 | <0.0005 | N/A | N/A | 12.1 | 16.1 | 18.1 |
| GDF-15 | Q99988 | <0.0001 | 569.4 | 657 | 917 | 1046 | 1247 |
| CD163 | Q86VB7 | <0.001 | 2027 | 2190 | 2615 | 2746 | 3265 |

Table 11B
Protein Markers for NAS Scores (NAS)

| Marker Name | UniProt | p value | NAS stages | | | | | | | |
|---|---|---|---|---|---|---|---|---|---|---|
| CD163 | Q86VB7 | <0.001 | 2429 | 1943 | 2363 | 2258 | 2676 | 2817 | 2918 | 3033 |

TABLE 11C
Protein Markers for Lobular Inflammation (LI)

| | | | Lobular | Lobular | Lobular |
|---|---|---|---|---|---|
| | | | inflam- | inflam- | inflam- |
| | | | mation | mation | mation |
| Marker Name | UniProt | p value | stage 0 | stage 1 | stage 2 |
| GDF-15 | Q99988 | <0.05 | 623.2 | 858.6 | 1024 |
| CD163 | Q86VB7 | <0.001 | 2002 | 2413 | 2846 |

TABLE 11D
Protein Markers for Hepatic Ballooning (HB)

| | | | Hepatic | Hepatic | Hepatic |
|---|---|---|---|---|---|
| | | | ballooning | ballooning | ballooning |
| Marker Name | UniProt | p value | stage 0 | stage 1 | stage 2 |
| GDF-15 | Q99988 | <0.005 | 630.9 | 774.7 | 1039 |
| CD163 | Q86VB7 | <0.0001 | 1966 | 2473 | 2863 |

Table 12: Protein markers for monitoring clinical improvements

TABLE 12
Protein Biomarkers for Monitoring Improvements in
NASH related clinical endpoints

| | | | Median | | |
|---|---|---|---|---|---|
| | | | percent | Median | |
| | | | CHG at | percent | |
| | | | W24 in | CHG at | |
| Marker | | Clinical | Non- | W24 in | |
| Name | UniProt | endpoints | improver | improver | p value |
| YKL-40 | P36222 | NAS score | 0.5 | −23.9 | <0.05 |
| YKL-40 | P36222 | Steatosis | 10.8 | −22.9 | <0.05 |
| FAP | Q12884 | Steatosis | 2.4 | −6.2 | <0.05 |
| MCM2 | P49736 | Steatosis | 4.3 | −11.8 | 0.08 |
| SCF | P21583 | Ballooning | 1.9 | 7.4 | <0.05 |
| YKL-40 | P36222 | Inflammation | 4.3 | −22 | <0.05 |
| FAP | Q12884 | Inflammation | 1.5 | −12.6 | <0.05 |
| MCM2 | P49736 | MRE | 4.3 | −7.9 | 0.06 |
| YKL-40 | P36222 | MRIPDFF | 6.5 | −22 | <0.05 |
| EMILIN1 | Q9Y6C2 | MRIPDFF | −2.8 | −16.6 | <0.05 |
| FAP | Q12884 | MRIPDFF | 1.2 | −17.5 | <0.05 |

### Example 5. Algorithms Using Noninvasive Tests can Accurately Identify Patients with Advanced Fibrosis Due to NASH: Data from STELLAR Clinical Trials

There is a major unmet need for accurate, readily available noninvasive tests (NITs) to identify patients with advanced fibrosis (F3-F4) due to NASH. The goal of this example was to evaluate sequential NITs to minimize the requirement for biopsy and improve accuracy over use of single tests.

Methods:

The STELLAR studies (NCT03053050 and NCT03053063) enrolled NASH patients with bridging fibrosis (F3) or compensated cirrhosis (F4). Baseline liver biopsies were centrally read using the NASH CRN fibrosis classification and noninvasive markers of fibrosis, including the Fibrosis-4 (FIB-4) index, Enhanced Liver Fibrosis (ELF) test, and FibroScan® (FS) were measured. The performance of these tests to discriminate advanced fibrosis was evaluated using AUROCs with 5-fold cross-validation repeated 100×. Thresholds were obtained by maximizing specificity given ≥85% sensitivity (and vice versa). The cohort was divided (80%/20%) into evaluation/validation sets. The evaluation set was further stratified 250× into training and test sets (66%/33%). Optimal thresholds were derived as average across training sets, and applied sequentially (FIB-4 followed by ELF and/or FS) to the validation set.

Results:

All screened and enrolled patients with available liver histology (N=3202, 71% F3-F4) and NIT results were included in the analysis. Using thresholds derived from STELLAR study data, FIB-4 followed by FS or ELF test in those with indeterminate FIB-4 values (1.23 to 2.1) demonstrated good performance characteristics while minimizing frequency of indeterminate values to as low as 13% (Table). Using published NIT thresholds resulted in similar findings (data not shown). Adding a third test (FIB-4 then ELF then FS) reduced the rate of indeterminate results to 8%. Misclassification occurred at rates similar to biopsy (15-21%). The majority of misclassifications (63-81%) were false negatives; among false positive cases (19-27% of misclassifications) up to 70% had F2 fibrosis.

Conclusion:

In these large, global, phase 3 trials with newly derived thresholds optimized for the STELLAR trials, FIB-4 followed by ELF and/or FS nearly eliminated the need for liver biopsy and accurately identified patients with advanced fibrosis due to NASH with misclassification rates similar to liver biopsy. Further validation of these findings in additional cohorts is planned.

TABLE 13
Diagnostic Performance of NITs to Discriminate Advanced Fibrosis (F3-F4)

| | | Sample | | | | |
|---|---|---|---|---|---|---|
| Test* | Cohort | Size | Sensitivity | Specificity | Indeterminate | Misclassified** |
| FIB-4 (1.23, 2.1) | Train + Test | 2496 | 85% | 85% | 32% | 15% |
| | Validation | 627 | 83% | 89% | 32% | 15% |
| ELF (9.35, 10.24) | Train + Test | 2536 | 85% | 85% | 29% | 15% |
| | Validation | 637 | 85% | 85% | 29% | 15% |
| FS (9.6 kPa, 14.53 kPa) | Train + Test | 1408 | 85% | 86% | 28% | 15% |
| | Validation | 357 | 82% | 88% | 25% | 17% |
| FIB-4 (1.23, 2.1) | Train + Test | 2542 | 79% | 81% | 13% | 20% |
| then ELF (9.35, 10.24) | Validation | 638 | 78% | 82% | 13% | 21% |
| FIB-4 (1.23, 2.1) then | Train + Test | 2509 | 82% | 85% | 20% | 17% |
| FS (9.6 kPa, 14.53 kPa) | Validation | 632 | 78% | 87% | 20% | 19% |

*Lower value represents optimal threshold to exclude advanced fibrosis, higher value to diagnose advanced fibrosis, with in-between values classified as indeterminate
**Proportion of misclassified patients relative to total sample size including indeterminate zone

### Example 6. Identification of NASH Associated Serum Metabolites and Diagnosis Biomarkers Using an OWLiver® Metabolomics Platform

This example was conducted to identify serum metabolites correlated with NASH disease severities, and to evaluate the performance of selected metabolite panels as classifiers for advance fibrosis, cirrhosis, active NASH and cryptogenic cirrhosis. The technology used here, referred to as OWLiver® metabolomics, is commercially available from OWL Metabolomics (Bizkaia, Spain) and is described in Barr et al, J Proteome Res. 2010 September 3; 9(9):4501-12 and Mayo et al., Hepatol Commun. 2018 May 4; 2(7):807-820.

Study Design:

Samples: 279 serum samples from either healthy individuals or NAFLS/NASH subjects with various degree of biopsy-confirmed liver fibrosis (F0-F4) were included in current study (Table 14).

TABLE 14
Serum samples included in the study

| Fibrosis stage | Healthy | F0 | Fl | F2 | F3 | F4 | Total |
|---|---|---|---|---|---|---|---|
| No. of samples | 30 | 39 | 45 | 55 | 52 | 58 | 279 |

Assay platform: Metabolomics profiling in serum were performed using mass spectrometry (MS) based approach at OWL Metabolomics.

Data Analysis:

Trend analysis was performed for categorical NASH parameters(fibrosis, NAS & components) and correlation analysis was performed for continuous variables (MQC, ELF, MRE, and MRI-PDFF). Univariate logistic regression (LR) with 5-fold cross-validation 100 times was used to select markers with AUROC>=0.7. Wilcoxon rank sum test was used to select markers with BH-adjusted p-value<0.05. Overlapping markers from the two analyses were selected as classifiers.

The analysis included metabolites in the families as shown in Table 15.

| Family | | Number |
|---|---|---|
| Phospho-ethanolamine (PE) | MEMAPE | 6 |
| | MAPE | 18 |
| | MEPE | 2 |
| | DAPE | 8 |
| Phospho-cholines (PC) | MAPC | 35 |
| | MEPC | 12 |
| | DAPC | 27 |
| | MEMAPC | 18 |
| Phospho-inositol (PI) | DAPI | 2 |
| | MAPI | 5 |
| | TAG | 50 |
| | DAG | 6 |
| | FFA | 21 |
| | Bile acids | 8 |
| | Acylcarnitines | 2 |
| | Ceramides | 3 |
| | Cholesterol Esters | 10 |
| | Sphingolipids | 22 |
| | Amino acids | 24 |
| | TAG | 50 |

MEMAPE (1-ether, 2-acylglycerophosphoethanolamine), MAPE (monoacylglycerophosphoethanolamine), MEPE (1-monoetherglycerophosphoethanolamine), DAPE (diacylglycerophosphoethanolamines), MAPC (1-monoacylglycerophosphocholine), MEPC (1-monoetherglycerophosphocholine), DAPC (diacylglycerophosphocholines), MEMAPC (1-ether, 2-acylglycerophosphocholine), DAPI (diacylglycerophosphoinositol), MAPI (monoacylglycerophosphoinositol), TAG: (triglycerides); DAG (diacylglycerides); FFA (free fatty acids)

Metabolites significantly associated with one or more of the fibrosis stages are listed in the tables below.

TABLE 15
Metabolites significantly (p < 0.01) associated with
CRN fibrosis stage in NASH subjects

| Phosphocholines (PC) | | Phosphoethanol | Amino acids | Sphingolipids |
|---|---|---|---|---|
| PC(0:0/14:0) | PC(O-16:0/0:0) | amine (PE) | Valine | SM(d18:1/17:0) |
| PC(16:0/0:0) | PC(O-18:1/0:0) | PE(0:0/16:0) | Taurine | SM(d18:1/18:0) |
| PC(17:0/0:0) | PC(O-20:0/0:0) | PE(0:0/18:0) | Leucine | SM(d18:1/22:0) |
| PC(18:0/0:0) | PC(O-20:1/0:0) | PE(P-16:1/0:0) | Glutamine | SM(d18:1/23:0) |
| PC(14:0/20:4) | PC(O-20:2/0:0) | PE(P-18:2/0:0) | Lysine | SM(d18:2/14:0) |
| PC(15:0/20:4) | PC(O-22:0/0:0) | LPE(20:5) | Histidine | SM(d18:2/16:0) |
| PC(16:0/20:5) | PC(O-22:1/0:0) | PE(16:0/18:2) | Sarcosine | SM(d18:2/20:0) |
| PC(18:2/20:4) | PC(O-24:2/0:0) | PE(18:1/18:2) | Phe-Phe | SM(31:1) |
| PC(18:3/18:3) | PC(P-16:0/0:0) | PE(22:5/0:0) | Glutamic Acid | SM(36:2) |
| PC(0:0/18:2) | PC(P-18:0/0:0) | PE(22:5/0:0) | Phenylalanine | SM(38:1) |
| PC(18:2/0:0) | PC(P-18:1/0:0) | Phosphoinositol | Ceramides | SM(39:1) |
| PC(0:0/20:2) | PC(P-20:2/0:0) | (PI) | Cer(d18:1/22:0) | SM(42:1) |
| PC(0:0/22:4) | PC(O-16:0/22:4) | LPI(18:0) | Cer(d18:1/24:0) | SM(42:3) |
| PC(22:4/0:0) | PC(O-20:0/20:4) | LPI(18:2) | Cer(d18:1/16:0) | Bile acids |
| PC(0:0/22:5) | PC(O-22:0/20:4) | LPI(20:3) | Free fatty acids | Chenodeoxycholic |
| PC(0:0/22:5) | PC(O-34:0) | LPI(20:4) | 20:3n-3 | acid |
| PC(22:5/0:0) | PC(16:0/16:0) | LPI(22:6) | 20:3n-9 | Glycocholic acid |
| PC(22:5/0:0) | PC(16:0/18:0) | PI(18:0/18:2) | 20:4n-6 | Taurocholic acid |
| PC(40:5) | PC(18:0/18:2) | Cholesterol | 20:5n-3 | Taurodeoxycholic |
| Acylcarnitine | Triacyl- | Esters | 22:6n-3 | acid |
| AC(12:0) | glycerides | ChoE(20:3) | | Taurochenodeoxy- |
| | TG(44:2) | ChoE(20:4) | | cholic acid |
| | TG(53:0) | ChoE(20:5) | | |
| | | ChoE(22:6) | | |

Underlined and bold: negative correlation; otherwise positive correlation.

TABLE 16
Metabolites significantly (p < 0. 001) associated with NAS score

| Phosphocholines (PC) | | Phosphoethanolamine (PE) |
|---|---|---|
| PC(16:0/16:0) | PC(O-16:0/14:0) | PE(0:0/16:1) |
| PC(16:0/18:0) | PC(O-16:0/16:0) | Spingolipids |
| PC(20:0/18:2) | PC(O-34:1) | SM(d18:0/18:0) |
| PC(40:8) | PC(P-16:0/20:4) | Amino acids |
| PC(0:0/20:0) | PC(O-16:0/22:4) | Valine |
| PC(20:0/0:0) | PC(O-18:0/20:4) | Methionine |
| PC(0:0/20:1) | PC(O-20:0/20:4) | Ceramides |
| PC(20:1/0:0) | PC(O-22:1/20:4) | Cer(d18:1/16:0) |
| PC(19:0/0:0) | PC(O-22:0/20:4) | |
| | PC(O-24:1/20:4) | |

Underlined and bold: negative correlation; otherwise positive correlation.

TABLE 17
Metabolites significantly(p < 0.001) associated with Steatosis

| Phosphocholines (PC) | | Phosphoethanolamine (PE) | Amino acids |
|---|---|---|---|
| PC(16:0/16:0) | PC(O-16:0/14:0) | PE(0:0/16:1) | Valine |
| PC(16:0/18:0) | PC(O-16:0/16:0) | Phosphoinositol (PI) | Taurine |
| PC(18:0/18:2) | PC(P-16:0/18:2) | PI(18:0/20:4) | Leucine |
| PC(20:0/18:2) | PC(P-16:0/20:4) | Sphingolipids | Methionine |
| PC(19:0/0:0) | PC(O-16:0/22:4) | SM(d18:1/16:0) | Tyrosine |
| PC(0:0/20:0) | PC(P-17:0/20:4) | Cholesterol Esters | L-Citrulline |
| PC(20:0/0:0) | PC(O-18:0/20:4) | ChoE(18:3) | Ceramides |
| PC(0:0/20:1) | PC(P-18:0/20:4) | Bile acids | Cer(d18:1/16:0) |
| PC(20:1/0:0) | PC(O-20:0/20:4) | Glycocholic acid | Diacylglycerides |
| PC(0-34:0) | PC(O-22:1/20:4) | Taurocholic acid | DG(32:2) |
| PC(0-34:1) | PC(O-22:0/20:4) | Taurochenodeoxycholic | |
| PC(40:8) | PC(O-24:1/20:4) | acid | |
| | PC(14:0/20:4) | | |
| | PC(0:0/14:0) | | |

Underlined and bold: negative correlation; otherwise positive correlation.

TABLE 18
Metabolites significantly (p < 0.001)
associated with Hepatocyte Ballooning
Phosphocholines (PC)

| | |
|---|---|
| PC(O-22:1/20:4) | PC(20:0/18:2) |
| PC(O-24:1/20:4) | PC(O-16:0/14:0) |
| PC(19:0/0:0) | PC(40:8) |
| PC(O-34:1) | PC(0:0/20:0) |
| PC(O-22:0/20:4) | PC(O-16:0/20:4) |
| PC(O-20:0/20:4) | PC(O-18:0/20:4) |
| PC(O-16:0/16:0) | PC(16:0/18:0) |
| PC(P-16:0/20:4) | |

Underlined and bold: negative correlation; otherwise positive correlation.

TABLE 19
Metabolites significantly(p < 0. 01)
associated with Lobular inflammation
Sphingolipids
SM(38:0)
SM(d18:0/18:0)
SM(d18:0/22:0)

Underlined and bold: negative correlation; otherwise positive correlation.

TABLE 20
Metabolites significantly(p < 0. 001) associated with
Morphometric Quantification of Collagen(MQC)

| | | |
|---|---|---|
| Phosphocholines | Phosphoethanolamine | Bile acids |
| (PC) | (PE) | Taurocholic acid |
| PC(16:0/16:0) | PE(22:5/0:0) | Taurochenodeoxycholic |
| PC(18:0/18:1) | PE(22:6/0:0) | acid |
| PC(O-16:0/14:0) | PE(18:1/18:2) | Glycocholic acid |
| PC(O-16:0/16:0) | Amino acids | Free fatty acids |
| PC(O-16:0/22:4) | Taurine | 20:3n-3 |
| PC(O-18:0/20:4) | Lysine | 20:4n-6 |
| PC(O-20:0/20:4) | Methionine | Sphingolipids |
| PC(O-22:0/20:4) | Phenylalanine | SM(36:2) |
| PC(O-22:1/20:4) | Tyrosine | SM(d18:1/18:0) |
| PC(O-24:1/20:4) | Phe-Phe | |
| PC(O-34:0) | | |
| PC(O-34:1) | | |

Underlined and bold: negative correlation; otherwise positive correlation.

TABLE 21
Metabolites significantly (p < 0.001) associated
with Enhanced Liver Fibrosis (ELF) score

| Phosphocholines (PC) | | Phosphoethanolamine (PE) |
|---|---|---|
| PC(14:0120:4) | PC(O-16:0/18:2) | PE(0:0/22:6) |
| PC(0:0/20:1) | PC(O-16:0/20:4) | PE(22:6/0:0) |
| PC(16:0/16:0) | PC(O-16:0/22:4) | Amino acids |
| PC(16:0/18:0) | PC(O-18:0/20:4) | Methionine |
| PC(18:0/18:1) | PC(O-20:0/20:4) | Phenylalanine |
| PC(18:0/18:2) | PC(O-22:0/20:4) | Tyrosine |
| PC(18:2/0:0) | PC(O-22:1/20:4) | Bile acids |
| PC(20:0/18:2) | PC(O-24:1/20:4) | Taurocholic acid |
| PC(20:1/0:0) | PC(O-34:0) | Taurodeoxycholic acid |
| PC(O-16:0/14:0) | PC(O-34:1) | Taurochenodeoxycholic |
| PC(O-16:0/16:0) | PC(O-38:4) | acid |

| | |
|---|---|
| Ceramides | Glycocholic acid |
| Cer(d18:1/16:0) | Glycodeoxycholic acid |

Underlined and bold: negative correlation; otherwise positive correlation.

TABLE 22
Metabolites significantly (p < 0. 05) associated
with Magnetic Resonance Elastography (MRE)

| | |
|---|---|
| Phosphocholines (PC) | Bile acids |
| PC(0:0/18:2) | Taurochenodeoxycholic acid |
| PC(18:2/0:0) | Glycocholic acid |
| Sphingolipids | Triacylglycerides |
| SM(d18:1/17:0) | TG(46:0) |
| SM(d18:1/18:0) | TG(56:1) |
| | TG (58:2) |
| | TG (58:3) |

Underlined and bold: negative correlation; otherwise positive correlation.

TABLE 23
Metabolites significantly (p < 0.05) associated with Magnetic
Resonance Imaging-Proton Density Fat Fraction (MRI-PDFF)

| | | |
|---|---|---|
| Triacylglycerides | Phosphocholines (PC) | Amino acids |
| TG(44:0) | PC(18:0/0:0) | Glycine |
| TG(46:0) | PC(18:1/0:0) | Serine |
| TG(47:1) | PC(0:0/20:1) | Asparagine |
| TG(48:0) | PC(20:1/0:0) | Aspartic Acid |
| TG(48:1) | PC(22:6/0:0) | Glutamine |
| TG(49:0) | PC(17:0/0:0) | Valine |
| TG(49:1) | PC(0:0/17:1) | Acylcarnitines |
| TG(50:0) | PC(18:3/0:0) | AC(8:0) |
| TG(50:1) | Phosphoethanolamine (PE) | Sphingolipids |
| TG (51:1) | PE(0:0/16:1) | SM(d18:0/16:0) |
| TG(52:0) | | |
| TG(52:1) | | |
| TG(56:1) | | |

Underlined and bold: negative correlation; otherwise positive correlation.

TABLE 24
Metabolites significantly (p < 0.01) different in the serum form subjects
with cryptogenic cirrhosis compared to cirrhosis subjects with NASH

| Phosphocholines (PC) | | Phosphoethanolamine | Triacylglycerides | | Diacylglycerides |
|---|---|---|---|---|---|
| PC(0:0/14:0) | PC(0-34:1) | PE(0:0/16:0) | TG(44:0) | TG(50:0) | DG(32:1) |
| PC(14:0/20:4) | PC(O-16:0/18:2) | PE(0:0/16:1) | TG(44:1) | TG(50:1) | DG(32:2) |
| PC(16:0/16:0) | PC(P-16:0/18:2) | PE(0:0/20:3) | TG(46:0) | TG(50:2) | DG(34:1) |
| PC(16:0/18:0) | PC(P-16:0/20:4) | PE(20:3/0:0) | TG(46:1) | TG(52:0) | DG(34:2) |
| PC(20:0/20:4) | PC(P-18:0/20:4) | PE(20:4/0:0) | TG(48:0) | TG(52:1) | Amino acids |
| PC(0:0/20:0) | PC(O-20:0/20:4) | Cholesterol Esters | TG(48:1) | TG(54:0) | Glutamic Acid |
| PC(20:0/0:0) | PC(O-22:1/20:4) | ChoE(16:0) | TG(48:2) | TG(54:1) | Valine |
| PC(0:0/20:1) | Sphingolipids | ChoE(18:1) | TG(49:0) | TG(56:1) | Aspartic Acid |
| PC(O-16:0/16:0) | SM 38:0 | ChoE(18:3) | TG(49:1) | TG(56:2) | L-Citrulline |

Underlined and bold: negative correlation; otherwise positive correlation.

FIG. 19 shows common metabolite markers are present between different NASH phenotypes. Advance fibrosis (F>=3) alone: 17 metabolites; cirrhosis (F=4) alone: 30 metabolites; cryptogenic cirrhosis (F=4; no steatosis) alone: 34 metabolites; active NASH NAS score>=5 alone: 29 metabolites.

TABLE 25
Classifiers for diagnosis of advanced fibrosis and/or cirrhosis
Advance fibrosis (F3/4 vs F0-2); AUROC >= 0.70; p < 0.05

| | |
|---|---|
| Phosphocholines (PC) | Phosphoethanolamine (PE) |
| PC(0:0/22:5) | PE(P-16:1/0:0) |
| PC(O-16:0/0:0) | Bile acids |
| PC(O-18:1/0:0) | GCA |
| PC(O-20:0/0:0) | TCA |
| PC(O-20:1/0:0) | TCDCA |
| PC(O-20:2/0:0) | Phospho-inositol (PI) |
| PC(O-22:0/0:0) | LPI(18:2) |
| PC(O-22:1/0:0) | Amino acids |
| PC(P-16:0/0:0) | Taurine |
| PC(P-18:0/0:0) | |
| PC(P-18:1/0:0) | |

Underlined and bold: lower in F3/4; otherwise higher in F3/4

Cirrhosis (F4 vs F0-3); AUROC >= 0.70; p < 0.05

| | | |
|---|---|---|
| Phosphocholines | Phosphoethanolamine | Amino acids |
| (PC) | (PE) | Lysine |
| PC(14:0/20:4) | PE(P-16:1/0:0) | Taurine |
| PC(O-16:0/0:0) | PE(18:1/18:2) | Phenylalanine |
| PC(P-16:0/0:0) | Bile acids | Phe-Phe |
| PC(16:0/16:0) | GCA Glycocholic acid | Tyrosine |
| PC(16:0/18:0) | TCA Taurocholic acid | Ceramides |
| PC(O-16:0/16:0) | Taurochenodeoxycholic | Cer (d18:1/16:0) |
| PC(O-16:0/22:4) | acid | Free fatty acids |
| PC(O-18:0/20:4) | Sphingolipids | 20:3n-3 |
| PC(O-20:0/20:4) | SM(36:2) | 20:4n-6 |
| PC(O-22:0/20:4) | SM(38:1) | |
| PC(O-22:1/20:4) | SM(d18:1/18:0) | |
| PC(O-34:0) | SM(d18:1/22:0) | |
| PC(O-34:1) | | |

Underlined and bold: lower in F3/4; otherwise higher in F3/4

TABLE 26
Classifiers for Cryptogenic cirrhosis (Cryptogenic cirrhosis (CC)
vs NASH with cirrhosis (NC); AUROC >= 0.70; p < 0. 05)

| | | |
|---|---|---|
| Phosphocholines | Phosphoethanolamine | Triacylglycerides |
| (PC) | (PE) | TG(46:0) |
| PC(0:0/14:0) | PE(0:0/20:3) | TG(46:1) |
| PC(14:0/20:4) | PE(20:3/0:0) | TG(48:0) |
| PC(0:0/20:0) | Diacylglycerides | TG(48:1) |
| PC(0:0/20:1) | DG 32:1 | TG(48:2) |
| PC(16:0/16:0) | DG 32:2 | TG(50:0) |
| PC(20:0/0:0) | DG 34:1 | TG(50:1) |
| PC(20:0/20:4) | DG 34:2 | TG(50:2) |
| PC(O-16:0/16:0) | Amino acids | TG(52:1) |
| PC(O-16:0/18:2) | Aspartic Acid | Cholesterol Ester |
| PC(O-20:0/20:4) | Glutamic Acid | ChoE(16:0) |
| PC(O-22:1/20:4) | Valine | ChoE(18:1) |
| PC(O-34:1) | L-Citrulline | ChoE(18:3) |
| PC(P-16:0/18:2) | | |
| PC(P-16:0/20:4) | | |
| PC(P-18:0/20:4) | | |

Underlined and bold: lower in CC; otherwise higher in CC

TABLE 27
Classifiers for active NASH (NAS >= 5; AUROC >= 0.70; p < 0.05)

| Phosphocholines (PC) | | Phosphoethanolamine (PE) |
|---|---|---|
| PC(0:0/20:0) | PC(O-18:0/20:4) | PE(18:1/18:2) |
| PC(0:0/20:1) | PC(O-20:0/20:4) | PE(22:6/0:0) |
| PC(16:0/16:0) | PC(O-22:0/20:4) | PE(P-16:0/18:2) |
| PC(16:0/18:0) | PC(O-22:1/20:4) | Sphingolipids |
| PC(19:0/0:0) | PC(O-24:1/20:4) | SM(d18:0/18:0) |
| PC(20:0/0:0) | PC(O-34:0) | Amino acids |
| PC(O-16:)/14:0) | PC(O-34:1) | Methionine |
| PC(O-16:0/16:0) | PC(P-16:0/18:2) | Tyrosine |
| PC(O-16:0/18:2) | PC(P-16:0/20:4) | Ceramides |
| PC(O-16:0/20:4) | PC(P-17:0/20:4) | Cer(d18:1/16:0) |
| PC(O-16:0/22:4) | PC(P-18:0/20:4) | |

Underlined and bold: lower in NAS>=5; otherwise higher in NAS>=5

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for determining the stage of liver fibrosis in a human subject in need thereof, comprising:
measuring the expression levels of one or more proteins, selected from Tables 1A-1F and 11A-11D in a biological sample isolated from the human subject; and
determining the stage of liver fibrosis in the human subject based on the expression levels.

2. The method of claim 1, wherein the determination comprises comparing the expression levels to reference levels.

3. The method of claim 2, wherein the reference levels are obtained from a human subject not suffering from liver fibrosis.

4. The method of any one of claims 1-3, wherein the expression levels of at least two proteins are measured.

5. The method of claim 4, wherein the expression levels of at least three proteins are measured.

6. The method of any one of claims 1-5, wherein the expression levels are protein expression levels or mRNA expression levels.

7. The method of any one of claims 1-6, wherein the biological sample is a serum sample.

8. The method of any one of claims 1-7, further prescribing or administering to the human subject a suitable therapy according to the determined stage of liver fibrosis.

9. The method of claim 8, wherein the therapy is selected from the group consisting of a(n) ACE inhibitor, Acetyl CoA carboxylase (ACC) inhibitor, Adenosine A3 receptor agonist, Adiponectin receptor agonist, AKT protein kinase inhibitor, AMP-activated protein kinases (AMPK), Amylin receptor agonist, Angiotensin II AT-1 receptor antagonist, Apoptosis signal-regulating kinase 1(ASK1) inhibitor, Autotaxin inhibitors, Bioactive lipid, Calcitonin agonist, Caspase inhibitor, Caspase-3 stimulator, Cathepsin inhibitor, Caveolin 1 inhibitor, CCR2 chemokine antagonist, CCR3 chemokine antagonist, CCR5 chemokine antagonist, Chloride channel stimulator, CNR1 inhibitor, Cyclin D1 inhibitor, Cytochrome P450 7A1 inhibitor, DGAT1/2 inhibitor, Dipeptidyl peptidase IV inhibitor, Endosialin modulator, Eotaxin ligand inhibitor, Extracellular matrix protein modulator, Farnesoid X receptor agonist, Fatty acid synthase inhibitors, FGF1 receptor agonist, Fibroblast growth factor (FGF-15, FGF-19, FGF-21) ligands, Galectin-3 inhibitor, Glucagon receptor agonist, Glucagon-like peptide 1 agonist, G-protein coupled bile acid receptor 1 agonist, Hedgehog (Hh) modulator, Hepatitis C virus NS3 protease inhibitor, Hepatocyte nuclear factor 4 alpha modulator (HNF4A), Hepatocyte growth factor modulator, HMG CoA reductase inhibitor, IL-10 agonist, IL-17 antagonist, Ileal sodium bile acid cotransporter inhibitor, Insulin sensitizer, integrin modulator, intereukin-1 receptor-associated kinase 4 (IRAK4) inhibitor, Jak2 tyrosine kinase inhibitor, Klotho beta stimulator, ketohexokinase inhibitors such as PF-06835919, 5-Lipoxygenase inhibitor, Lipoprotein lipase inhibitor, Liver X receptor, LPL gene stimulator, Lysophosphatidate-1 receptor antagonist, Lysyl oxidase homolog 2 inhibitor, Matrix metalloproteinases (MMPs) inhibitor, MEKK-5 protein kinase inhibitor, Membrane copper amine oxidase (VAP-1) inhibitor, Methionine aminopeptidase-2 inhibitor, Methyl CpG binding protein 2 modulator, MicroRNA-21(miR-21) inhibitor, a mitochondrial uncoupler such as nitazoxanide, Myelin basic protein stimulator, NACHT LRR PYD domain protein 3 (NLRP3) inhibitor, NAD-dependent deacetylase sirtuin stimulator, NADPH oxidase inhibitor (NOX), Nicotinic acid receptor 1 agonist, P2Y13 purinoceptor stimulator, PDE 3 inhibitor, PDE 4 inhibitor, PDE 5 inhibitor, PDGF receptor beta modulator, Phospholipase C inhibitor, PPAR alpha agonist, PPAR delta agonist, PPAR gamma agonist, PPAR gamma modulator, Protease-activated receptor-2 antagonist, Protein kinase modulator, Rho associated protein kinase inhibitor, Sodium glucose transporter-2 inhibitor, SREBP transcription factor inhibitor, STAT-1 inhibitor, Stearoyl CoA desaturase-1 inhibitor, Suppressor of cytokine signalling-1 stimulator, Suppressor of cytokine signalling-3 stimulator, Transforming growth factor β (TGF-β), Transforming growth factor β activated Kinase 1 (TAK1), Thyroid hormone receptor beta agonist, TLR-4 antagonist, Transglutaminase inhibitor, Tyrosine kinase receptor modulator, GPCR modulator, nuclear hormone receptor modulator, WNT modulators, or YAP/TAZ modulator.

10. The method of any one of claims 1-9, wherein the genes are selected from Tables 2A-2F and 11A-D.

11. The method of any one of claims 1-9, wherein the expression levels are measured for at least three genes selected from the group consisting of:
Complement component 7 (C7),
Collectin Kidney 1 (CL-K1),
Insulin-like growth factor binding protein 7 (IGFBP7),
Spondin-1(RSPO1),
Interleukin 5 receptor subunit alpha (IL5-Ra),
Matrix metallopeptidase 7 (MMP-7), and
Thrombospondin-2 (TSP2).

12. The method of claim 11, wherein the expression levels are measured for at least four genes selected from the group.

13. The method of claim 11, wherein the expression levels are measured for at least five genes selected from the group.

14. The method of claim 11, wherein the expression levels are measured for at least six genes selected from the group.

15. The method of claim 11, wherein the expression levels are measured for all seven genes selected from the group.

16. The method of any one of claims 11-15, wherein the determined stage of the liver fibrosis is advanced fibrosis.

17. A method for providing biological information for diagnosing liver fibrosis in a human subject, comprising measuring the expression levels of two or more genes, selected from Tables 1A-1F and 11A-11D, in a biological sample isolated from the human subject.

18. The method of claim 17, comprising measuring the expression levels of three or more genes selected from Tables 1A-1F and 11A-11D.

19. The method of claim 17, comprising measuring the expression levels of three or more genes selected from the group consisting of:
Complement component 7 (C7),
Collectin Kidney 1 (CL-K1),
Insulin-like growth factor binding protein 7 (IGFBP7),
Spondin-1(RSPO1),
Interleukin 5 receptor subunit alpha (IL5-Ra),
Matrix metallopeptidase 7 (MMP-7), and
Thrombospondin-2 (TSP2).

20. The method of any one of claims 17-19, wherein measurement is carried out for no more than 20 genes.

21. A method for providing biological information for determining the CRN (Nonalcoholic Steatohepatitis Clinical Research Network) fibrosis stage in a human subject, comprising measuring the expression levels of two or more genes, selected from Tables 1A and 11A, in a biological sample isolated from the human subject.

22. A method for providing biological information for determining the Ishak fibrosis stage in a human subject, comprising measuring the expression levels of two or more genes, selected from Table 1B, in a biological sample isolated from the human subject.

23. A method for providing biological information for determining the NAS (nonalcoholic fatty liver disease (NAFLD) activity score) in a human subject, comprising measuring the expression levels of two or more genes, selected from Tables 1C and 11B, in a biological sample isolated from the human subject.

24. A method for providing biological information for characterizing steatosis in a human subject, comprising measuring the expression levels of two or more genes, selected from Table 1D, in a biological sample isolated from the human subject.

25. A method for providing biological information for characterizing lobular inflammation in a human subject, comprising measuring the expression levels of two or more genes, selected from Tables 1E and 11C, in a biological sample isolated from the human subject.

26. A method for providing biological information for characterizing hepatic ballooning in a human subject, comprising measuring the expression levels of two or more genes, selected from Tables 1F and 11D, in a biological sample isolated from the human subject.

27. The method of any one of claims 21-26, further comprising making a diagnosis based on the biological information.

28. The method of claim 27, further comprising prescribing or administering to the human subject a therapy according to the diagnosis.

29. A method for assessing the effect of a treatment in a patient suffering from liver fibrosis and having received the treatment, comprising
measuring the expression levels of one or more genes, selected from Tables 3A-3D and 12, in a biological sample isolated from the patient; and
assessing the effect of the treatment by comparing the expression levels to baseline expression levels obtained from the patients prior to the treatment.

30. The method of claim 29, wherein the expression levels are further compared to control expression levels obtained from a control patient, wherein the control patient also suffers from liver fibrosis and having received the treatment.

31. The method of claim 29 or 30, further comprising continuing the treatment if the effect is assessed to be positive, or changing or terminating the treatment if the effect is assessed to be negative.

32. The method of any one of claims 29-31, wherein the patient has received the treatment for at least about 24 weeks.

33. The method of any one of claims 29-32, comprising measuring the expression levels of at least two of the genes selected from Tables 3A-D and 12.

34. The method of any one of claims 29-32, comprising measuring the expression levels of at least three of the genes selected from Tables 3A-D and 12.

35. The method of any one of claims 29-32, comprising measuring the expression levels of at least three genes selected from the group consisting of:
Phosphatase and tensin homolog (PTEN),
CD70,
Caspase 2,
Cathepsin H (CTSH),
Sphingosine N-acyltransferase (LAG-1),
Pyridoxal kinase (PDXK), and
Glucocorticoid-induced TNFR-related protein (GITR).

36. The method of claim 35, comprising measuring the expression levels of at least four genes selected from the group.

37. The method of claim 35, comprising measuring the expression levels of at least five genes selected from the group.

38. The method of claim 35, comprising measuring the expression levels of at least six genes selected from the group.

39. The method of claim 35, comprising measuring the expression levels of all seven genes selected from the group.

40. A method for providing biological information for assessing the effect of a treatment in a patient suffering from liver fibrosis and having received the treatment, comprising measuring the expression levels of two or more genes, selected from Tables 3A-D and 12, in a biological sample isolated from the patient.

41. The method of claim 40, comprising measuring the expression levels of three or more genes selected from Tables 3A-3D and 12.

42. The method of claim 40, comprising measuring the expression levels of three or more genes selected from the group consisting of:
Phosphatase and tensin homolog (PTEN),
CD70,
Caspase 2,
Cathepsin H (CTSH),
Sphingosine N-acyltransferase (LAG-1),
Pyridoxal kinase (PDXK), and
Glucocorticoid-induced TNFR-related protein (GITR).

43. The method of any one of claims 40-42, wherein measurement is carried out for no more than 20 genes.

44. A method for providing biological information for assessing whether a liver fibrosis patient exhibits improvement on steatosis following a treatment, comprising measuring the expression levels of two or more genes, selected from Tables 3A and 12, in a biological sample isolated from the human subject.

45. A method for providing biological information for assessing whether a liver fibrosis patient exhibits improvement on lobular inflammation following a treatment, comprising measuring the expression levels of two or more genes, selected from Table 3B, in a biological sample isolated from the human subject.

46. A method for providing biological information for assessing whether a liver fibrosis patient exhibits improvement on hepatic ballooning following a treatment, comprising measuring the expression levels of two or more genes, selected from Table 3C, in a biological sample isolated from the human subject.

47. A method for providing biological information for assessing whether a liver fibrosis patient exhibits improvement on CRN fibrosis stage following a treatment, comprising measuring the expression levels of two or more genes, selected from Tables 3D and 12, in a biological sample isolated from the human subject.

48. The method of any one of claims 44-47, further comprising making an assessment based on the biological information.

49. The method of claim 48, further comprising continuing, adjusting, or discontinuing the treatment according to the assessment.

50. A method for treating liver fibrosis in a human subject in need thereof, comprising administering to the human subject an effective amount of a liver fibrosis therapy,
wherein the human subject has undergone an analysis which measures the expression levels of one or more genes, selected from Tables 1A-1F and 11A-11D, in a biological sample isolated from the human subject, which analysis determines that the human subject suffers from liver fibrosis; and
wherein the liver fibrosis therapy is selected from the group consisting of a(n) ACE inhibitor, Acetyl CoA carboxylase (ACC) inhibitor, Adenosine A3 receptor agonist, Adiponectin receptor agonist, AKT protein kinase inhibitor, AMP-activated protein kinases (AMPK), Amylin receptor agonist, Angiotensin II AT-1 receptor antagonist, Apoptosis signal-regulating kinase 1(ASK1) inhibitor, Autotaxin inhibitors, Bioactive lipid, Calcitonin agonist, Caspase inhibitor, Caspase-3 stimulator, Cathepsin inhibitor, Caveolin 1 inhibitor, CCR2 chemokine antagonist, CCR3 chemokine antagonist, CCR5 chemokine antagonist, Chloride channel stimulator, CNR1 inhibitor, Cyclin D1 inhibitor, Cytochrome P450 7A1 inhibitor, DGAT1/2 inhibitor, Dipeptidyl peptidase IV inhibitor, Endosialin modulator, Eotaxin ligand inhibitor, Extracellular matrix protein modulator, Farnesoid X receptor agonist, Fatty acid synthase inhibitors, FGF1 receptor agonist, Fibroblast growth factor (FGF-15, FGF-19, FGF-21) ligands, Galectin-3 inhibitor, Glucagon receptor agonist, Glucagon-like peptide 1 agonist, G-protein coupled bile acid receptor 1 agonist, Hedgehog (Hh) modulator, Hepatitis C virus NS3 protease inhibitor, Hepatocyte nuclear factor 4 alpha modulator (HNF4A), Hepatocyte growth factor modulator, HMG CoA reductase inhibitor, IL-10 agonist, IL-17 antagonist, Ileal sodium bile acid cotransporter inhibitor, Insulin sensitizer, integrin modulator, intereukin-1 receptor-associated kinase 4 (IRAK4) inhibitor, Jak2 tyrosine kinase inhibitor, Ketohexokinase inhibitors; Klotho beta stimulator, ketohexokinase inhibitors such as PF-06835919, 5-Lipoxygenase inhibitor, Lipoprotein lipase inhibitor, Liver X receptor, LPL gene stimulator, Lysophosphatidate-1 receptor antagonist, Lysyl oxidase homolog 2 inhibitor, Matrix metalloproteinases (MMPs) inhibitor, MEKK-5 protein kinase inhibitor, Membrane copper amine oxidase (VAP-1) inhibitor, Methionine aminopeptidase-2 inhibitor, Methyl CpG binding protein 2 modulator, MicroRNA-21(miR-21) inhibitor, mitochondrial uncoupler such as nitazoxanide, Myelin basic protein stimulator, NACHT LRR PYD domain protein 3 (NLRP3) inhibitor, NAD-dependent deacetylase sirtuin stimulator, NADPH oxidase inhibitor (NOX), Nicotinic acid receptor 1 agonist, P2Y13 purinoceptor stimulator, PDE 3 inhibitor, PDE 4 inhibitor, PDE 5 inhibitor, PDGF receptor beta modulator, Phospholipase C inhibitor, PPAR alpha agonist, PPAR delta agonist, PPAR gamma agonist, PPAR gamma modulator, Protease-activated receptor-2 antagonist, Protein kinase modulator, Rho associated protein kinase inhibitor, Sodium glucose transporter-2 inhibitor, SREBP transcription factor inhibitor, STAT-1 inhibitor, Stearoyl CoA desaturase-1 inhibitor, Suppressor of cytokine signalling-1 stimulator, Suppressor of cytokine signalling-3 stimulator, Transforming growth factor β (TGF-β), Transforming growth factor β activated Kinase 1 (TAK1), Thyroid hormone receptor beta agonist, TLR-4 antagonist, Transglutaminase inhibitor, Tyrosine kinase receptor modulator, GPCR modulator, nuclear hormone receptor modulator, WNT modulators, or YAP/TAZ modulator.

51. A method for treating liver fibrosis in a human subject in need thereof, comprising administering to the human subject that suffers from liver fibrosis and has received a treatment,
wherein the human subject has undergone an analysis which measures the expression levels of one or more genes, selected from Tables 2A-2D and 11A-11D, in a biological sample isolated from the human subject, which analysis determines that the human subject exhibits improvements at a clinical endpoint following the treatment; and
wherein the anti-liver fibrosis therapy is selected from the group consisting of a(n) ACE inhibitor, Acetyl CoA carboxylase (ACC) inhibitor, Adenosine A3 receptor agonist, Adiponectin receptor agonist, AKT protein kinase inhibitor, AMP-activated protein kinases (AMPK), Amylin receptor agonist, Angiotensin II AT-1 receptor antagonist, Apoptosis signal-regulating kinase 1(ASK1) inhibitor, Autotaxin inhibitors, Bioactive lipid, Calcitonin agonist, Caspase inhibitor, Caspase-3 stimulator, Cathepsin inhibitor, Caveolin 1 inhibitor, CCR2 chemokine antagonist, CCR3 chemokine antagonist, CCR5 chemokine antagonist, Chloride channel stimulator, CNR1 inhibitor, Cyclin D1 inhibitor, Cytochrome P450 7A1 inhibitor, DGAT1/2 inhibitor, Dipeptidyl peptidase IV inhibitor, Endosialin modulator, Eotaxin ligand inhibitor, Extracellular matrix protein modulator, Farnesoid X receptor agonist, Fatty acid synthase inhibitors, FGF1 receptor agonist, Fibroblast growth factor (FGF-15, FGF-19, FGF-21) ligands, Galectin-3 inhibitor, Glucagon receptor agonist, Glucagon-like peptide 1 agonist, G-protein coupled bile acid receptor 1 agonist, Hedgehog (Hh) modulator, Hepatitis C virus NS3 protease inhibitor, Hepatocyte nuclear factor 4 alpha modulator (HNF4A), Hepatocyte growth factor modulator, HMG CoA reductase inhibitor, IL-10 agonist, IL-17 antagonist, Ileal sodium bile acid cotransporter inhibitor, Insulin sensitizer, integrin modulator, intereukin-1 receptor-associated kinase 4 (IRAK4) inhibitor, Jak2 tyrosine kinase inhibitor, Klotho beta stimulator, ketohexokinase inhibitors such as PF-06835919, 5-Lipoxygenase inhibitor, Lipoprotein lipase inhibitor, Liver X receptor, LPL gene stimulator, Lysophosphatidate-1 receptor antagonist, Lysyl oxidase homolog 2 inhibitor, Matrix metalloproteinases (MMPs) inhibitor, MEKK-5 protein kinase inhibitor, Membrane copper amine oxidase (VAP-1) inhibitor, Methionine aminopeptidase-2 inhibitor, Methyl CpG binding protein 2 modulator, MicroRNA-21(miR-21) inhibitor, Mitochondrial uncoupler such as nitazoxanide, Myelin basic protein stimulator, NACHT LRR PYD domain protein 3 (NLRP3) inhibitor, NAD-dependent deacetylase sirtuin stimulator, NADPH oxidase inhibitor (NOX), Nicotinic acid receptor 1 agonist, P2Y13 purinoceptor stimulator, PDE 3 inhibitor, PDE 4 inhibitor, PDE 5 inhibitor, PDGF receptor beta modulator, Phospholipase C inhibitor, PPAR alpha agonist, PPAR delta agonist, PPAR gamma agonist, PPAR gamma modulator, Protease-activated receptor-2 antagonist, Protein kinase modulator, Rho associated protein kinase inhibitor, Sodium glucose transporter-2 inhibitor, SREBP transcription factor inhibitor, STAT-1 inhibitor, Stearoyl CoA desaturase-1 inhibitor, Suppressor of cytokine signalling-1 stimulator, Suppressor of cytokine signalling-3 stimulator, Transforming growth factor β (TGF-β), Transforming growth factor β activated Kinase 1 (TAK1), Thyroid hormone receptor beta agonist, TLR-4 antagonist, Transglutaminase inhibitor, Tyrosine kinase receptor modulator, GPCR modulator, nuclear hormone receptor modulator, WNT modulators, or YAP/TAZ modulator.
